(19) United States
(12) Patent Application Publication
(10) Pub. No.: US 2011/0306541 A1
(43) Pub. Date: Dec. 15, 2011

(54) METHODS FOR TREATING HCV

(75) Inventors: William E. Delaney, IV, Foster City, CA (US); William A. Lee, Los Altos, CA (US); David W. Oldach, Chapel Hill, NC (US); Franck Rousseau, Durham, NC (US)

(73) Assignee: Gilead Sciences, Inc., Foster City, CA (US)

(21) Appl. No.: 13/158,168

(22) Filed: Jun. 10, 2011

Related U.S. Application Data
(60) Provisional application No. 61/353,460, filed on Jun. 10, 2010.

(51) Int. Cl. A61K 38/06; A61P 31/14; A61K 31/7056

(52) U.S. Cl. 514/4.3; 514/43

(57) ABSTRACT

This invention relates to combinations of therapeutic molecules useful for treating hepatitis C virus infection. The present invention relates to methods, uses, dosing regimens, and compositions.

### CROSS-REFERENCE TO RELATED APPLICATIONS

Priority is claimed to U.S. Provisional Application No. 61/353,460, filed 10 Jun. 2010, herein incorporated by reference in its entirety for all purposes.

### FIELD OF THE INVENTION

This invention relates to combinations of therapeutic molecules useful for treating hepatitis C virus infection. The present invention relates to methods, uses, dosing regimens, and compositions.

### BACKGROUND OF THE INVENTION

Hepatitis is a disease occurring throughout the world. Hepatitis is generally of viral nature, although, if considered a state of chronic inflammation of the liver, there are other known, non-infectious causes. Viral hepatitis is by far the most common form of hepatitis. The U.S. Centers for Disease Control has estimated that at least 1.8% of the U.S. population has serologic evidence of HCV infection, in the majority of cases associated with chronic active infection. HCV is a positive-stranded RNA virus belonging to the Flaviviridae family and has closest relationship to the pestiviruses that include hog cholera virus and bovine viral diarrhea virus.

The HCV genome is a single-stranded, positive-sense RNA of about 9,600 by coding for a polyprotein of 3009-3030 amino acids, which is cleaved co- and post-translationally by cellular and two viral proteinases into mature viral proteins (core, E1, E2, p7, NS2, NS3, NS4A, NS4B, NS5A, NS5B). The structural proteins, E1 and E2, are believed to be embedded into a viral lipid envelope and form stable heterodimers. The structural core protein is believed to interact with the viral RNA genome to form the nucleocapsid. The nonstructural proteins designated NS2 to NS5 include proteins with enzymatic functions involved in virus replication and protein processing including a polymerase, protease, and helicase. HCV replicates through the production of a complementary negative-strand RNA template.

HCV is a genetically diverse virus. Within a single infected patient, many variant viruses can be identified, leading to the description ‘viral swarm’, or viral quasispecies. Within the global human population, HCV is also genetically diverse, with at least 6 major ‘genotypes’ identified (Genotypes 1-6), and numerous subtypes (i.e., HCV Genotype 1a and 1b). HCV genotypes are defined by genomic phylogenetic analysis, and diagnosed (in a given patient) by HCV RNA sequence-based diagnostic assays.

The main route of infection with HCV is blood exposure. The magnitude of the HCV infection as a health problem is illustrated by the prevalence among high-risk groups. For example, in some surveys, 60% to 90% of hemophiliacs and more than 80% of intravenous drug abusers in western countries had chronic HCV infection. For intravenous drug abusers, the prevalence varies from about 28% to 80% depending on the population studied. The proportion of new HCV infections associated with blood or blood product transfusion has been markedly reduced due to pharmaceutical advances and widespread use of sensitive serologic and RNA detection assays used to screen blood donors, however, a large cohort of aging, chronically infected persons is already established.

One available treatment for HCV infection is pegylated interferon-α (PEG-IFN α1a or PEG-IFN α1b), which is, under current treatment guidelines, administered weekly by subcutaneous injection for 24 to 48 weeks, dependent upon the HCV viral genotype being treated. Although greater than 50% of patients with Genotype 1 HCV infection may be expected to have suppression of HCV viremia at the completion of 48 weeks therapy, a significant proportion of these patients will have viral relapse. Accordingly, a Sustained Virologic Response (SVR, defined as HCV RNA negativity 24 weeks post treatment cessation, and considered tantamount to ‘cure’) is only achieved in 30-40% of Genotype 1 HCV infections treated with PEG-IFN alone. In addition, treatment with PEG-IFN+RBV is not well tolerated, with an adverse event profile that includes flu-like symptoms, thrombocytopenia, anemia, and serious psychiatric side effects. While treatment with the current standard of care is suboptimal, many patients are precluded from ever starting therapy due to comorbidities common in HCV-infected populations, including psychiatric disorders, advanced liver disease, and substance abuse.

Ribavirin is a nucleoside analog antiviral drug. Ribavirin is typically taken orally (by mouth) twice a day. The exact mechanism for ribavirin is unknown. However, it is believed that when ribavirin enters a cell it is phosphorylated; it then acts as an inhibitor of inosine 5′-monophosphate dehydrogenase (IMPDH). IMPDH inhibitors such as ribavirin reduce the intracellular synthesis and storage of guanine, a nucleotide “building block” necessary for DNA and RNA production, thus inhibiting viral replication. IMPDH inhibitors also interfere with the reproduction of rapidly proliferating cells and cells with a high rate of protein turnover. Treatment with ribavirin monotherapy has little effect on HCV RNA levels, but is associated with a decline in serum alanine transferase (ALT). This observation suggests that ribavirin may not be acting as an antiviral agent, but rather as a modulator of immune system function. Ribavirin is only approved for use, for HCV infection, in combination with IFN.

Treatment with the combination of PEG-IFN plus ribavirin improves SVR rates over those observed with PEG-IFN alone, in large part due to reduction in the frequency of viral relapse at the cessation of therapy. Large clinical trial SVR rates for PEG-IFN/ribavirin treated patients with HCV Genotype 1 infection have ranged from 40-55%. At the present time, PEG-IFN/ribavirin therapy is considered the ‘standard-of-care’ treatment for chronic HCV infection. The standard of care is, however, expected to change rapidly in the near future with approval of direct acting antiviral agents which will, initially, be used in combination with PEG-IFN/ribavirin.

Current HCV therapy with ribavirin in combination with interferon is associated with an array of side effects, including but not limited to, flu-like effects such as fever, malaise, tachycardia, tachyphylaxis, chills, headache, arthralgias, and myalgias; neuropsychiatric effects such as fatigue, asthenia, drowsiness, lack of initiative, irritability, confusion, and apathy; behavioral, mood, and cognitive changes including depression; immunomodulatory effects such as autoimmune thyroiditis, hypothyroidism, and hyperthyroidism; cardiovascular effects, with both benign and severe cardiac manifestations reported and further including cardiac arrhythmias, supraventricular tachycardia and ventricular arrhythmias, as well as dilated cardiomyopathy and hypotension; renal effects such as proteinuria, including benign and nephritic, as well as interstitial nephritis and acute renal failure; hepatic effects; gastrointestinal effects including nausea, vomiting, dyspepsia, diarrhea, and abdominal pain; dermatological effects such as rashes including erythema multiforme, pruritus, hair loss, local erythema, psoriasis, and vitiligo; myelosuppression; hormonal and metabolic effects based upon a sustained increase in serum triglyceride levels and including diabetes mellitus; as well as ocular effects (retinopathy), interstitial fibrosis, and pneumonitis.

There is a continuing need therefore for the development of anti-viral agents.

### SUMMARY

One aspect of the present invention includes a dosing regimen for the treatment of HCV comprising: administering one or more anti-HCV compound or a pharmaceutically acceptable salt thereof; and ribavirin, but not one or more interferon.

Another aspect of the present invention includes a method for ameliorating one or more symptom of HCV infection in a human comprising: administering one or more anti-HCV compound or a pharmaceutically acceptable salt thereof; and ribavirin, without concurrent administration of one or more interferon. In this regard, the present invention does not foreclose the potential for dosing one or more interferon. Rather, the present invention may be used in conjunction with another therapy that, in fact, includes one or more interferon. An aspect of the present invention includes efficacious treatment of HCV with ribavirin without the need for one or more interferon.

Another aspect of the present invention includes a method for reducing viral load in a human diagnosed with HCV comprising: administering one or more anti-HCV compound or a pharmaceutically acceptable salt thereof; and ribavirin, but not one or more interferon.

Another aspect of the present invention includes a method for treating HCV in a human subject consisting essentially of administration of ribavirin in conjunction with one or more anti-HCV compound or a pharmaceutically acceptable salt thereof.

Another aspect of the present invention includes a method of ribavirin-based HCV therapy comprising: administering one or more anti-HCV compound of a pharmaceutically acceptable salt thereof; and avoiding administration of one or more interferon.

Another aspect of the present invention includes a method for reducing emergence of HCV quasispecies with resistance to coadministered oral antiviral agents comprising: administering one or more anti-HCV compound or a pharmaceutically acceptable salt thereof; and ribavirin, without concurrent administration of one or more interferon.

Similarly, another aspect of the present invention includes a composition for ameliorating one or more symptom of HCV infection in a human comprising: one or more anti-HCV compound or a pharmaceutically acceptable salt thereof; and ribavirin, without one or more interferon; as well as a composition for reducing viral load in a human diagnosed with HCV comprising: one or more anti-HCV compound or a pharmaceutically acceptable salt thereof; and ribavirin, but not one or more interferon; as well as a composition for treating HCV in a human subject consisting essentially of ribavirin in conjunction with one or more anti-HCV compound or a pharmaceutically acceptable salt thereof; as well as a composition for ribavirin-based HCV therapy comprising: one or more anti-HCV compound or a pharmaceutically acceptable salt thereof, with the proviso that said composition does not include one or more interferon; as well as a composition for reducing emergence of HCV quasispecies with resistance to coadministered oral antiviral agents comprising: one or more anti-HCV compound or a pharmaceutically acceptable salt thereof; and ribavirin, without one or more interferon.

Similarly, another aspect of the present invention includes use of: one or more anti-HCV compound or a pharmaceutically acceptable salt thereof; and ribavirin, without one or more interferon, in the manufacture of a medicament for ameliorating one or more symptom of HCV infection in a human; as well as use of: one or more anti-HCV compound or a pharmaceutically acceptable salt thereof; and ribavirin, but not one or more interferon, in the manufacture of medicament for reducing viral load in a human diagnosed with HCV; as well as use of ribavirin in conjunction with one or more anti-HCV compound or a pharmaceutically acceptable salt thereof in the manufacture of a medicament for treating HCV in a human subject, wherein said use does not include use of one or more interferon; as well as use of one or more anti-HCV compound of a pharmaceutically acceptable salt thereof, in the manufacture of a medicament for ribavirin-based HCV therapy, wherein said use avoids administration of one or more interferon.; as well as use of one or more anti-HCV compound or a pharmaceutically acceptable salt thereof; and ribavirin, without one or more interferon in the manufacture of a medicament for reducing emergence of HCV quasispecies with resistance to coadministered oral antiviral agents.

Another aspect of the present invention includes a combination comprising ribavirin; and one or more anti-HCV compound or a pharmaceutically acceptable salt thereof, which combination is substantially free of one or more interferon. In one embodiment, the combination may occur as separate dosage forms with each active ingredient, administered together or separate, sequentially or concurrently, and close in time or remote in time to each other.

Another aspect of the present invention includes a kit comprising: ribavirin; one or more anti-HCV compound; and instruction regarding a treatment regimen to treat, reduce viral load, or delay onset or progression of HCV wherein the treatment regimen includes administration of the one or more anti-HCV compound and ribavirin without administration of one or more interferon. In one embodiment, such a kit may also include packaging, such as a blister pack. Alternatively, such a kit may provide for individual prescription and dosing of each component as separately packaged pharmaceutics, but when combined with the instruction regarding a treatment regimen to treat, reduce viral load, or delay onset or progression of HCV, such is intended to be within the scope of the present invention.

Another aspect of the present invention includes a pharmaceutical composition comprising: ribavirin; one or more anti-HCV compound or a pharmaceutically acceptable salt thereof; and one or more pharmaceutically acceptable carrier. In one embodiment, the pharmaceutical composition may be a unitary dosage form.

In one embodiment of each aspect, the one or more anti-HCV compound is an NS3 protease inhibitor, NS4B inhibitor nucleoside, NS5B polymerase inhibitor, nonnucleoside NS5B polymerase inhibitor, NS5A inhibitor, an HCV entry inhibitor, an HCV assembly inhibitor or an HCV infectivity inhibitor. In a further embodiment of each aspect, the one or more anti-HCV compound is Compound 1:

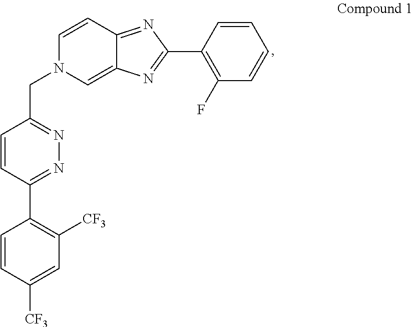

or a pharmaceutically acceptable salt thereof. In a further embodiment of each aspect, the present invention includes one or more additional anti-HCV compound or pharmaceutically acceptable salt thereof. In a further embodiment of each aspect, the one or more additional anti-HCV compound is Compound 2:

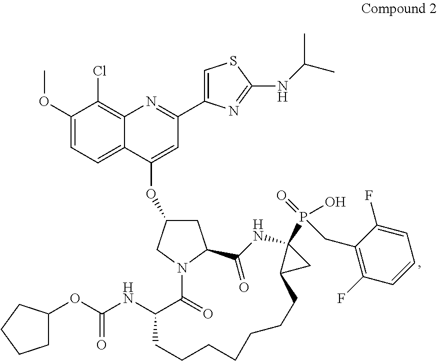

or a pharmaceutically acceptable salt thereof. In a further embodiment of each aspect, the present invention includes one or more additional anti-HCV compound or pharmaceutically acceptable salt thereof. In a further embodiment of each aspect, the one or more anti-HCV compound is one or more of Compounds 1-17 or any combination thereof. In a further embodiment of each aspect of the invention, the one or more anti-HCV compounds are Compound 1 and Compound 2. In a further embodiment of each aspect of the invention, the one or more anti-HCV compounds are Compound 1 and Compound 3. In another embodiment of each aspect, the combination of Compound 1, Compound 2, ribavirin and interferon is not present. In another embodiment of each aspect, the combination of Compound 1, Compound 2, and ribavirin is not present. In another embodiment of each aspect, the combination of one or more anti-HCV compounds is not Compound 1 and Compound 2.

The present invention includes combinations of aspects and embodiments, as well as preferences, as herein described throughout the present specification.

### DETAILED DESCRIPTION OF THE INVENTION

### Definitions

Unless stated otherwise, the following terms and phrases as used herein are intended to have the following meanings. The fact that a particular term or phrase is not specifically defined should not be correlated to indefiniteness or lacking clarity, but rather terms herein are used within their ordinary meaning. When trade names are used herein, applicants intend to independently include the tradename product and the active pharmaceutical ingredient(s) of the tradename product.

As used herein, ribavirin refers to:

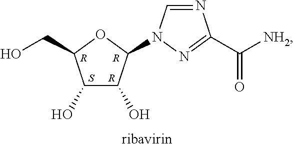

which may also be referred to as 1-β-D-ribofuranosyl-1H-1,2,4-Triazole-3-carboxamide, 1-β-D-ribofuranosyl-1,2,4-triazol-3-carboxyamide; 1-β-D-Ribofuranosyl-1,2,4-triazole-3-carboxamide; Copegus; ICN 1229; MegaRibavirin; NSC 163039; Ravanex; Rebetol; Ribamide; Ribamidil; Ribasphere; Ribavarin; Ribavirin; Tribavirin; Vilona; Viramid; Virazole; or Virizadole. In addition, as used herein ribavirin includes analogs of ribavirin, including taribavirin (Viramidine).

As used herein, Compound 1 refers to:

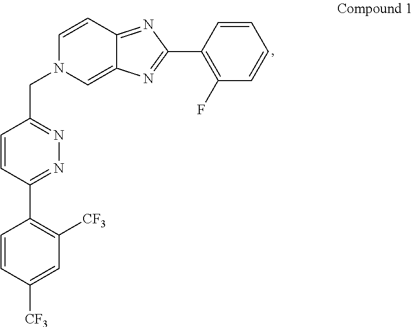

which may also be referred to as 5-((6-(2,4-bis(trifluoromethyl)phenyl)pyridazin-3-yl)methyl)-2-(2-fluorophenyl)-5H-imidazo[4,5-c]pyridine or 5H-imidazo[4,5-c]pyridine, 5-[[6-[2,4-bis(trifluoromethyl)phenyl]pyridazin-3-yl]methyl]-2-(2-fluorophenyl).

As used herein, Compound 2 refers to:

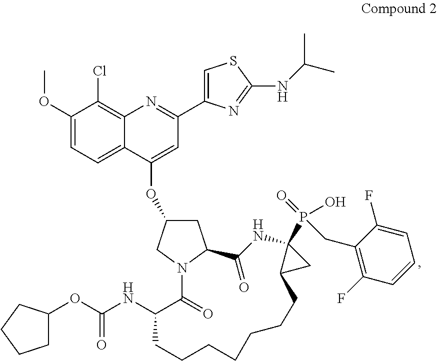

which may also be referred to as (2R,6S,13aR,14aS,16aS)-2-(8-chloro-2-(2-(isopropylamino)thiazol-4-yl)-7-methoxyquinolin-4-yloxy)-6-(cyclopentyloxycarbonylamino)-5,16-dioxooctadecahydrocyclopropa[e]pyrrolo[1,2-a][1,4]diazacyclopentadecin-14a-yl(2,6-diflurobenzyl)phosphinic acid.

The term “treating,” and grammatical equivalents thereof, when used in the context of treating a disease, means slowing or stopping the progression of a disease, or ameliorating at least one symptom of a disease, more preferably ameliorating more than one symptom of a disease. For example, treatment of a hepatitis C virus infection can include reducing the HCV viral load in an HCV infected human being, reducing plasma levels of ALT (alanine amino transferase) and/or reducing the severity of jaundice present in an HCV infected human being.

As will be appreciated by those skilled in the art, the compounds may exist in solvated or hydrated form. The scope of the present invention includes such forms.

Again, as will be appreciated by those skilled in the art, the compounds may be capable of esterification. The scope of the present invention includes esters and other physiologically functional derivatives. The scope of the present invention includes prodrug forms of the compounds herein described.

“Ester” means any ester of a compound in which any of the —COON functions of the molecule is replaced by a —C(O)OR function, or in which any of the —OH functions of the molecule are replaced with a —OC(O)R function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof.

The term “prodrug” as used herein refers to any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically active compound. Example of prodrugs include ester moieties, quaternary ammonium moieties, glycol moieties, and the like.

The compounds may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York.

Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or I meaning that the compound is levorotatory. A compound prefixed with (+) or d is dextrorotatory.

A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms. Typically, but not absolutely, the salts of the present invention are pharmaceutically acceptable salts. Salts encompassed within the term “pharmaceutically acceptable salts” refer to non-toxic salts of the compounds of this invention. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### Protecting Groups

In the context of the present invention, protecting groups include prodrug moieties and chemical protecting groups. Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene and Peter G. M. Wuts (John Wiley & Sons, Inc., New York, 1999, ISBN 0-471-16019-9) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below. Such groups include by way of example and not limitation, esters, amides, hydrazides, and the like.

### Metabolites of the Compounds of the Invention

Also falling within the scope of this invention are the in vivo metabolic products of the compounds described herein. Such products may result for example from the oxidation, reduction, hydrolysis, amidation, esterification, addition of glutathione and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled (e.g., C¹⁴ or H³) compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no anti-infective activity of their own.

### Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like.

The pH of the formulations ranges from about 3 to about 11, but is ordinarily about 7 to 10.

While it is possible for the active ingredients to be administered alone it may be preferable to present them as pharmaceutical formulations. The formulations of the invention, both for veterinary and for human use, comprise at least one active ingredient, together with one or more acceptable carriers and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

In yet another embodiment, the present application discloses pharmaceutical compositions comprising an anti-HCV compound, such as Compound 1 or Compound 2, or a pharmaceutically acceptable salt thereof, and a pharmaceutically acceptable carrier or excipient.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Combination Therapy, Including HCV Combination Therapy

In another embodiment, non-limiting examples of suitable combinations include combinations of one or more compounds with one or more additional therapeutic for HCV treatment including HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, HCV IRES inhibitors, pharmacokinetic enhancers, as well as other drugs for treating HCV. More specifically, one or more compounds of the present invention may be combined with one or more compounds selected from the group consisting of: (i) HCV NS3 protease inhibitors, e.g., boceprevir (SCH-503034, SCH-7), telaprevir (VX-950), VX-813, TMC-435 (TMC435350), ABT-450, ACH-1625, ACH-2684, BI-201335, BI-1230, MK-5172, MK-7009, SCH-900518, VBY-376, VX-500, GS-9256, GS-9451, BMS-605339, PHX-1766, AS-101, YH-5258, YH5530, YH5531, and ITMN-191 (R-7227); (ii) alpha-glucosidase 1 inhibitors, e.g., celgosivir (MX-3253), Miglitol, and UT-231B; (iii) hepatoprotectants, e.g., emericasan (IDN-6556), ME-3738, GS-9450 (LB-84451), silibilin, and MitoQ; (iv) nucleoside or nucleotide inhibitors of HCV NS5B polymerase, e.g., R1626, R7128 (R4048), IDX184, IDX-102, PSI-661, PSI-938, PSI-7851, PSI-7977, BCX-4678, valopicitabine (NM-283), MK-0608 and TMC649128; (v) non-nucleoside inhibitors of HCV NS5B polymerase, e.g., filibuvir (PF-868554), ABT-333, ABT-072, BI-207127, VCH-759, VCH-916, JTK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, and GS-9190; (vi) HCV NS5A inhibitors, e.g., ACH-2928, AZD-2836 (A-831), AZD-7295 (A-689), BMS-766, BMS-790052, BMS-824393, and PPI-461; (vii) TLR-7 agonists, e.g., imiquimod, 852A, GS-9524, ANA-773, ANA-975, AZD-8848 (DSP-3025), PF-04878691, and SM-360320; (viii) cyclophillin inhibitors, e.g., DEBIO-025, SCY-635, and NIM811; (ix) HCV IRES inhibitors, e.g., MCI-067; (x) pharmacokinetic enhancers, e.g., BAS-100, SPI-452, PF-4194477, TMC-41629, GS-9350, GS-9585, and roxythromycin; and (xi) other drugs for treating HCV, e.g., thymosin alpha 1 (Zadaxin), nitazoxanide (Alinea, NTZ), BIVN-401 (virostat), PYN-17 (altirex), KPE02003002, actilon (CPG-10101), GS-9525, KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, FK-788, and VX-497 (merimepodib).

While one aspect of the present invention includes use of ribavirin in combination with one or more anti-HCV compound or a pharmaceutically acceptable salt thereof, without the need for one or more interferon, an additional suitable combination includes an additional administration of one or more interferons in temporal relation to the administration of the present invention, such as: 1) interferons, e.g., pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), and belerofon.

In yet another embodiment, the present application discloses pharmaceutical compositions comprising a compound of the present invention, or a pharmaceutically acceptable salt thereof, in combination with at least one additional active agent, and a pharmaceutically acceptable carrier or excipient. In yet another embodiment, the present application provides a combination pharmaceutical agent with two or more therapeutic agents in a unitary dosage form. Thus, it is also possible to combine any compound of the invention with one or more other active agents in a unitary dosage form.

The combination therapy may be administered as a simultaneous or sequential regimen. When administered sequentially, the combination may be administered in two or more administrations.

Co-administration of a compound of the invention with one or more other active agents generally refers to simultaneous or sequential administration of a compound of the invention and one or more other active agents, such that therapeutically effective amounts of the compound of the invention and one or more other active agents are both present in the body of the patient.

Co-administration includes administration of unit dosages of the compounds of the invention before or after administration of unit dosages of one or more other active agents, for example, administration of the compounds of the invention within seconds, minutes, or hours of the administration of one or more other active agents. For example, a unit dose of a compound of the invention can be administered first, followed within seconds or minutes by administration of a unit dose of one or more other active agents. Alternatively, a unit dose of one or more other active agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of a compound of the invention.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. The effective dose of an active ingredient depends at least on the nature of the condition being treated, toxicity, whether the compound is being used prophylactically (lower doses) or against an active disease or condition, the method of delivery, and the pharmaceutical formulation, and will be determined by the clinician using conventional dose escalation studies. In one embodiment of the present invention, the combined amount of ribavirin and anti-HCV compound or a pharmaceutically acceptable salt thereof, optionally with one or more additional agent, is effective to treat HCV infection. The compounds may be administered together (e.g., in the form of a unit dosage, such as a tablet), or separately. If administered separately, each compound may be administered with the other(s) at the same time, or either before or after such administration of the other(s). Typically, the compounds are administered daily. In one embodiment, a daily dosage is administered in separate sub-doses, such as twice daily or three times per day.

By way of example, in the practice of the methods of this aspect of the invention, a daily amount of from 1.0 mg to 100 mg or 5 to 100 mg of Compound 1, or a pharmaceutically acceptable salt thereof, preferably from 30 mg to 50 mg, preferably from 20 mg to 40 mg, and preferably 40 mg and from 1000 mg to 1200 mg (divided doses) of ribavirin can be administered daily to a human being in need thereof. In one embodiment, an amount of Compound 2 or a pharmaceutically acceptable salt thereof, optionally with Compound 1 or a pharmaceutically acceptable salt thereof, is administered in a daily amount of from 25 mg to 800 mg, preferably from 50 mg to 400 mg, preferably from 60 mg to 300 mg, preferably from 70 mg to 200 mg, and preferably 75 mg. Dosage of 150 mg of Compound 2 or a pharmaceutically acceptable salt thereof administered once or twice daily may also be used. In one embodiment, the target range for exposure of Compound 2 is 40 μg.hr/mL to 80 μg.hr/mL (corresponding to a dosage of 75 mg to 150 mg). In one embodiment, an amount of any one of Compound 3, 4, 5, 6, 7, 8, 9, 10, or 11, or a pharmaceutically acceptable salt thereof is administered, optionally with Compound 1 or a pharmaceutically acceptable salt thereof and optionally with Compound 2 or a pharmaceutically acceptable salt thereof, in a daily amount of 100 mg to 400 mg, preferably 200 mg. In one embodiment, an amount of Compound 1 or a pharmaceutically acceptable salt thereof is administered with Compound 3 or a pharmaceutically acceptable salt thereof. In one embodiment, Compound 3 would be administered in a dosage of 10-1000 mg or from 50-400 mg or from 100-400 mg or from 200-400 mg. Doses above 400 mg have been associated with more bilirubin elevations in some cases. The corresponding exposures (AUC) in this dosing range are 272.13 ng·h/ml (10 mg dose) to 48,401.48 ng·h/ml (1000 mg dose). In one embodiment, an amount of any one of Compounds 12-17 or a pharmaceutically acceptable salt thereof is also administered, optionally with any one of Compounds 1-11, or a pharmaceutically acceptable salt thereof, in a daily amount of 1 mg to 120 mg, preferably 10 mg to 60 mg, preferably 30 mg. In one embodiment, Compound 16 would be administered in a dosage of 3-300 mg or from 3-100 mg or from 10-90 mg or from 30-90 mg. The corresponding exposures are 32.3 ng·h/ml (3 mg dose), 1415.2 ng·h/ml (30 mg dose) and 4137.9 (90 mg dose), 11166.6 ng·h/ml (100 mg dose), 38900 ng·h/mL (300 mg dose).

Dosages for Compounds 1-17 that are co-administered may need to be adjusted to account for potential drug-drug interactions. For example, although it does not appear that Compound 1 affects drug metabolizing systems, Compound 2 appears to have the effect of increasing the exposure of Compound 1 approximately 2-3×. Therefore, a dose reduction (e.g. 2×-3×) of Compound 1 would be anticipated when Compound 1 is combined with Compound 2.

The course of treatment can extend, for example, from about 12 weeks to about 48 weeks, or such as, for example, from about 12 weeks to about 24 weeks.

### Methods of Treatment

As will be appreciated by those skilled in the art, when treating a viral infection such as HCV, such treatment may be characterized in a variety of ways and measured by a variety of endpoints. The scope of the present invention is intended to encompass all such characterizations.

The present invention includes a combination of therapeutically effective components to ameliorate at least one symptom of HCV infection in a human being. Thus, for example, in some HCV infected individuals a therapeutically effective amount of the combination is effective to reduce by a statistically significant amount the viral load of HCV viral particles present in the body of the infected person. Viral load can be measured, for example, by measuring plasma HCV RNA levels using, for example, the COBAS TaqMan HCV assay (Roche Molecular Systems). Typically, an HCV infected person who is treated with the combination in accordance with the present invention experiences an improvement in one or all of the symptoms associated with the HCV infection. For example, an HCV patient may experience an improvement in one or all of the following symptoms that can be associated with HCV infection: fever, headache, muscle aches, fatigue, loss of appetite, nausea, vomiting and diarrhea.

### SYNTHETIC EXAMPLES

### Example 1a

### Synthesis of 5-({6-[2,4-bis(trifluoromethyl)phenyl]pyridazin-3-yl}methyl)-2-(2-fluorophenyl)-5H-imidazo[4,5-c]pyridine

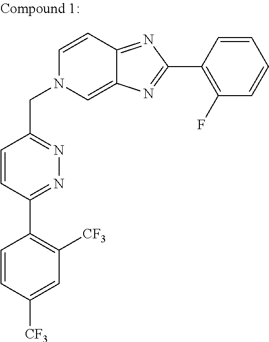

Compound 1 has the IUPAC name: 5-({6-[2,4-bis(trifluoromethyl)phenyl]pyridazin-3-yl}methyl)-2-(2-fluorophenyl)-5H-imidazo[4,5-c]pyridine, and the CAS name: 5H-imidazo[4,5-c]pyridine, 5-[[6-[2,4-bis(trifluoromethyl)phenyl]pyridazin-3-yl]methyl]-2-(2-fluorophenyl).

In this method for making Compound 1, dimethoxyethane or its related solvents, all having the general formula R¹OR²O(R⁴O)ₐR³ wherein each of R¹, R², R³, and R⁴ are independently selected from C₁-C₆ alkyl and a is 0 or 1, have been found to be particularly advantageous over the conventional solvent DMF. Typically, each of R¹, R², R³, and R⁴ are independently C₁-C₂ alkyl and usually a is 0.

As used herein, C₁-C₆ alkyl includes fully saturated primary, secondary, or tertiary hydrocarbon groups with 1 to 6 carbon atoms and thereby includes, but is not limited to methyl, ethyl, propyl, butyl, and the like.

### Step 1

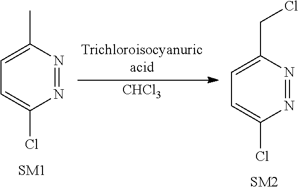

| Compound | MW | Amount | | mmoles | Equivalents |
|---|---|---|---|---|---|
| SM1 | 128.56 | 5 | g | 38.9 | 1 |
| TCCA | 232.41 | 3.62 | g | 15.6 | 0.4 |
| CHCl₃ | | 130 | mL | | |

To a solution of the commercially available starting material (SM1) in CHCl₃, trichloroisocyanuric acid (TCCA) was added at 60° C. Then the solution was stirred for 1.5 hrs, cooled, and filtered with HiFlo-Celite. The filtrate was concentrated and dried with vacuum. The yield was 5.037 g of SM2.

### Step 2

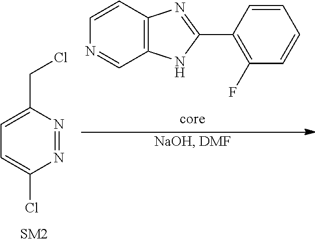

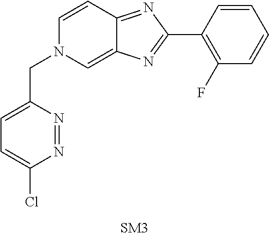

| Compound | MW | Amount | | mmoles | Equivalents |
|---|---|---|---|---|---|
| SM2 | 163 | 5.073 | g | 31.12 | 1 |
| Core | 213.2 | 6.635 | g | 31.12 | 1 |
| NaOH (10%) | 40 | 1.245 | g | 31.12 | 1 |
| DMF | | 320 | mL | | |

To a solution of the starting material designated as “core” (obtained as described below) in DMF (dimethylformamide), NaOH was added. Then SM2 (obtained as described in step 1) was dissolved in DMF (20 mL) and added to the solution slowly. The reaction was stirred for 3 hrs, was diluted with water and extracted with EtOAc. The organic layer was dried with Na2SO4. The solvent was removed and the product recrystallized with DCM (dichloromethane). The yield was 5.7 g of SM3.

### Step 3

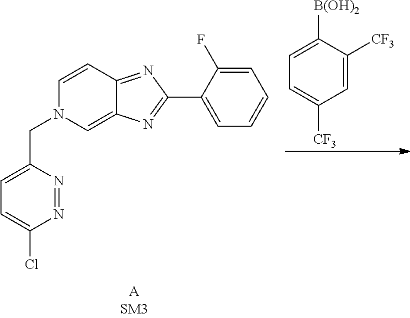

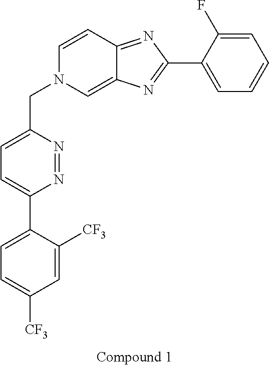

| Compound | MW | Amount | | Moles | Equivalents |
|---|---|---|---|---|---|
| SM3 | 453.79 | 95 | mg | 0.209 | 1 |
| DME | 500 μL | | | | |
| 2N aq. Na₂CO₃ | | 313 | μL | 0.626 | 3 |
| 2,4-bis-CF₃- | 257.93 | 80.9 | mg | 0.313 | 1.5 |
| phenylboronic | | | | | |
| acid | | | | | |
| Pd(PPh₃)₄ | 1155 | 12 | mg | 0.0104 | 0.05 |

The compound SM3 (obtained as described in step 2) was dissolved in dimethoxyethane (DME). To this solution was added 2,4-bis(trifluromethyl)phenylboronic acid and a 2N aq. Na₂CO₃ solution. To the resulting biphasic mixture was added Pd(PPh₃)₄ and the reaction was then heated at 80° C. for 72 hrs. The reaction was cooled to room temperature and filtered through Celite and the Celite washed with EtOAc. The filtrate was concentrated in vacuo. The residue was purified on 6 g SiO₂ using MeOH/CH₂Cl₂ to elute compound. The compound thus obtained was contaminated with PPh₃(O). The product was repurified on a 1 mm Chromatotron plate with 0 to 5% MeOH/CH₂Cl₂ in 1% steps. The pure fractions were combined and concentrated in vacuo, then dried on high vacuum for 12 hrs. 11.8 mg of the free base of compound (1) was obtained with no PPh₃ contamination.

¹H NMR (300 MHz, CD₃OD) δ 6.20 (s, 2), 7.32 (m, 3), 7.52 (m, 1), 7.78 (d, 1), 7.89 (d, 1), 7.95 (s, 2), 8.15 (m, 3), 8.35 (d, 1), 9.12 (s, 1); LC/MS M+H=518.

### Example 1b

### Synthesis of 5-({6-[2,4-bis(trifluoromethyl)phenyl]pyridazin-3-yl}methyl)-2-(2-fluorophenyl)-5H-imidazo[4,5-c]pyridine

This example is directed to an alternative method for making Compound 1. The following general schemes are instructive:

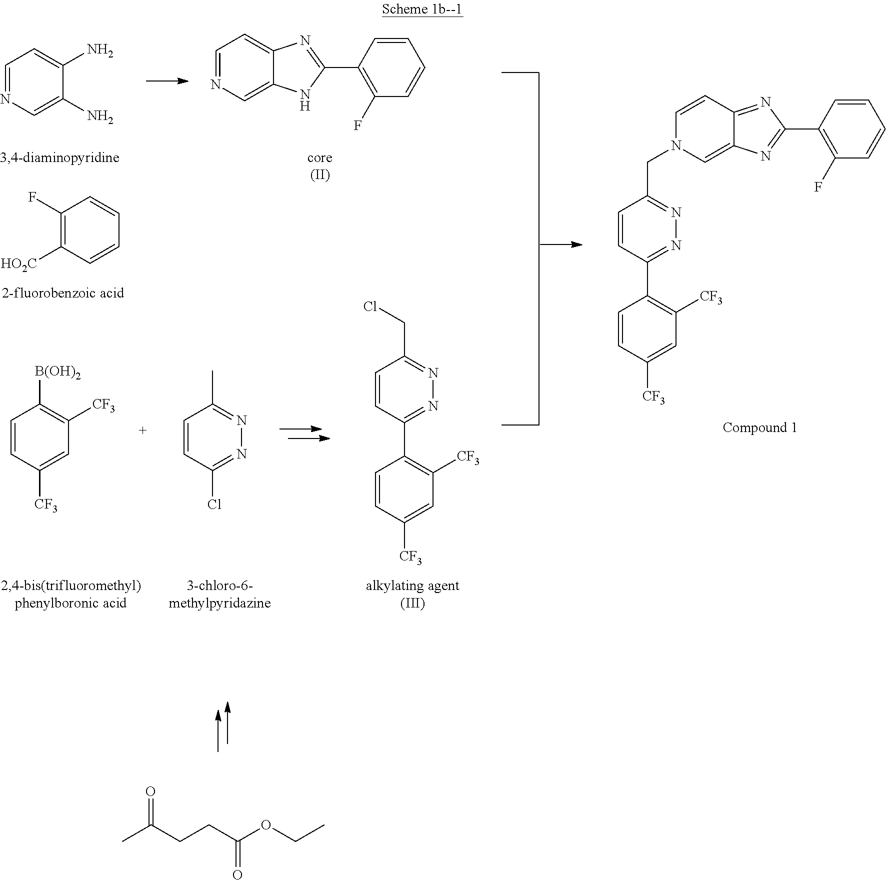

### Process Summary

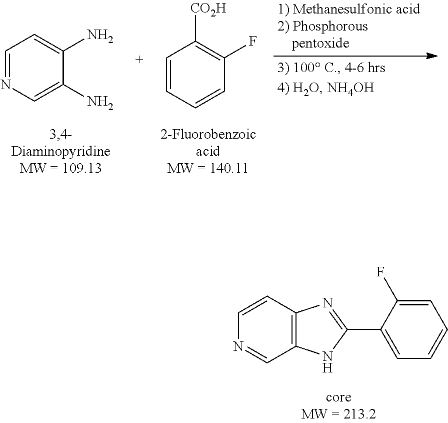

Methanesulfonic acid was added to 2-fluorobenzoic acid in a reactor with active cooling keeping T≦50° C. 3,4-Diaminopyridine was then added portion-wise to this cooled slurry, keeping T≦35° C. The contents of the reactor were then heated to 50° C. Phosphorus pentoxide was added in a single charge. The reaction was then heated at 90-110° C. for at least 3 hours. The reaction was sampled for completion by HPLC analysis. The reaction was cooled to ambient temperature and water was added portion-wise slowly to quench the reaction. The reaction was then diluted with water. Any insolubles were removed by filtration. The pH of the filtrate was adjusted to 5.5-5.8 with ammonium hydroxide. The reaction was allowed to self-seed and granulate for ˜4 hours at ambient temperature. The pH was then adjusted to 8.0-9.3 with ammonium hydroxide. The slurry was held at ambient temperature for at least 2 hours. The solids were isolated by filtration and washed with water, followed by IPE. The wet cake was dried in vacuo at not more than 60° C. until 51% water remains. The dry product is the compound designated as “core”.

| Summary of Materials | M.W. | Wt. Ratio | Mole ratio |
|---|---|---|---|
| 3,4-Diaminopyridine | 109.13 | 1.0 | 1.0 |
| 2-Fluorobenzoic acid | 140.11 | 1.4 | 1.1 |
| Methanesulfonic acid | 96.1 | 7.0 | 8.0 |
| Phosphorus pentoxide | 141.94 | 1.3 | 1.0 |
| Water | 18.02 | 40 | — |
| Isopropyl ether | 102.17 | 5.0 | — |
| Ammonium hydroxide | 35.09 | ~10 | — |

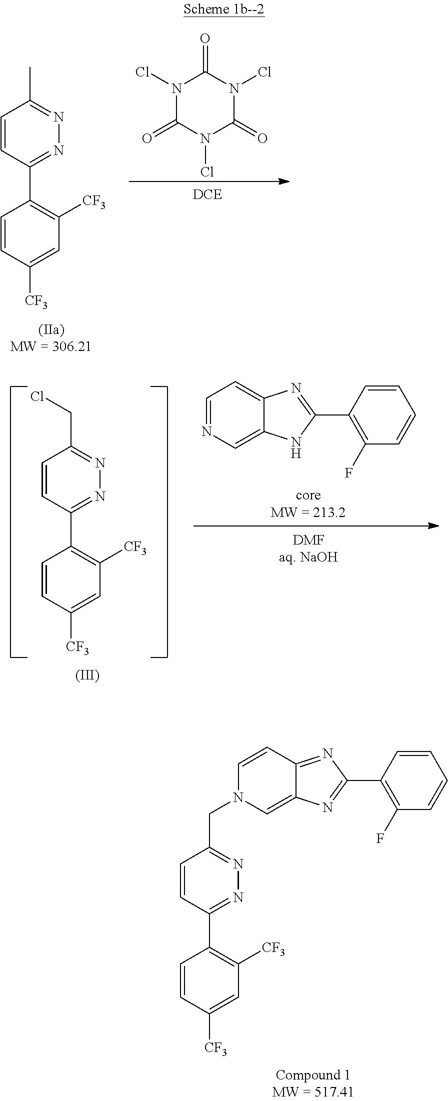

A solution of Compound IIa in 1,2-dichloroethane was heated to 40-45° C. Trichloroisocyanuric acid was added and the mixture was heated at 60-70° C. for at least 2 hours. The reaction was sampled for completion by HPLC analysis. The reaction was cooled to ambient temperature. Celite was added to absorb insolubles, then solids were removed by filtration. The filtrate was washed with 0.5 N sodium hydroxide solution. The organic layer was concentrated to lowest stirrable volume and displaced with DMF. The compound designated as “core” and 10% aqueous sodium hydroxide solution were added. The reaction was stirred at ambient temperature for at least 8 hours. The reaction was sampled for completion by HPLC analysis. An additional 10% charge of 10% sodium hydroxide solution was added to the reaction. The reaction was then charged into water to isolate the crude product, compound (1). After granulating for at least 1 hour, the solids were isolated and washed with water and isopropyl ether.

The wet cake was recrystallized from ethyl acetate to afford low melt (˜220° C.) Compound 1 (polymorph I). The wet cake was then reslurried in ethyl acetate in the presence of less than about 0.5% water to obtain the high melt (˜236° C.) Compound 1 (polymorph II).

The solids were collected by filtration and washed with ethyl acetate. The wet cake was dried in vacuo at not more than 60° C. to obtain the dry crystalline polymorph II.

| Summary of Materials | M.W. | Wt. Ratio | Mole ratio |
|---|---|---|---|
| 3-chloro-6-methylpyridazine | 128.56 | 1.0 | 1.0 |
| 2,4bis(trifluromethyl)phenylboronic | 257.93 | 4.0 | 2.0 |
| acid | | | |
| X-Phos | 476.72 | 0.18 | 0.05 |
| Palladium acetate | 224.49 | 0.04 | 0.025 |
| 1,2-Dimethoxyethane | 90.12 | 16.7 | — |
| Potassium carbonate | 138.21 | 2.15 | 2.0 |
| Water | 18.02 | 7.8 | — |
| Copper iodide | 190.45 | 0.037 | 0.025 |
| Celite | — | 0.25 | — |
| Heptane | 100.2 | 22.4 | — |

### Example 2

### Preparation of Compound 2

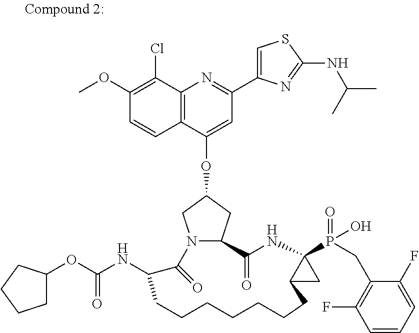

1. Synthesis and Resolution of Diethyl (1S, 2R)-1-amino-2-ethenylcyclopropane-1-phosphonate dibenzoyl-L-tartaric Acid Salt

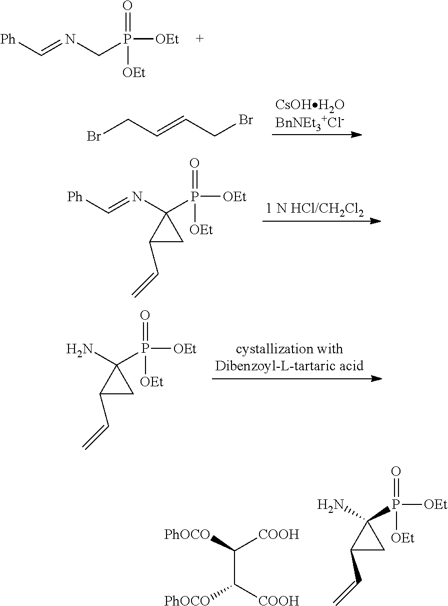

A solution of diethyl-(N-benzylideneaminomethyl)-phosphonate (50 g, 196 mmol), trans-1,4-dibromo-2-butene (50 g, 235 mmol), and benzyltriethylammonium chloride (4.5 g, 19.6 mmol) in dichloromethane (1.0 L) was stirred at room temperature using a mechanical stirrer when cesium hydroxide monohydrate (82 g, 490 mmol) was added. The resulting mixture was stirred for 18 hr after which another portion of cesium hydroxide monohydrate (82 g, 490 mmol) was added. The resulting mixture was stirred for 24 hr. The salts were then filtered off through a Celite 521 pad and the filtrate was allowed to stir with 1 N aq. HCl at room temperature for 3 h. The resulting mixture was filtered through another Celite 521 pad and the two phases of the filtrate were separated. The organic fraction was extracted with 1 N aq. HCl (250 mL×1). The aqueous fractions were washed with dichloromethane (250 mL×1) and the combined aq. fractions were stirred with ethyl acetate (500 mL) while 84 g (1 mol) of NaHCO₃ was added cautiously, followed by excess NaCl until saturated. After the resulting mixture was filtered through a Celite 521 pad to remove excess NaCl and some black tar, the two layers were separated and the aqueous fraction was extracted further with ethyl acetate (250 mL×2). The organic extracts were washed with a saturated NaCl solution (250 mL×1), combined, dried (MgSO₄), and concentrated to obtain ˜16.5-17 g of the crude amine.

The crude amine was partially purified by column chromatography using 165-170 g of silica gel by eluting with ethyl acetate (100%, ˜500 mL), followed by 5% methanol in ethyl acetate (˜1200 mL). The product containing fractions were pooled and concentrated, which resulted 11.5-12 g of partially purified amine.

To this amine was added a solution of 18.8-19.6 g (1 mole eq.) of dibenzoyl-L-tartaric acid in 151.5-158 mL of acetonitrile (5 times the amount of the salt). The mixture was heated until it became a solution and cooled slowly at room temperature to obtain solids. After overnight, the solids were collected by filtration and washed with acetonitrile. The solids were recrystallized from the same amount of acetonitrile again at room temperature to afford 11.5 g of optically pure salt.

¹H NMR (300 MHz, CD₃OD) δ 8.14 (br, 2H), 8.11 (d, J=1.2 Hz, 2H), 7.64 (tt, J=7.5 and 1.2 Hz, 2H), 7.51 (br t, J=7.5 Hz, 4H), 5.94 (s, 2H), 5.82 (dt, J=17.1 and 9.9 Hz, 1H), 5.32 (dd, J=17.1 and 1.2 Hz, 1H), 5.13 (dd, J=10.5 and 1.2 Hz, 1H), 4.11-4.26 (m, 4H), 2.11 (m, 1H), 1.33-1.47 (m, 2H), 1.37 (dt, J=10.2 and 7.2 Hz, 6H); ³¹P NMR (121.4 MHz, CD₃OD) δ 22.55.

Analytical: The optical purity of the amine can be determined by ³¹P NMR of Mosher's amide in DMSO-d₆. The recrystallized material (25 mg) was dissolved in a mixture of saturated aq. NaHCO₃ (5 mL) and saturated aq. NaCl (5 mL), and the free amine was extracted using dichloromethane (10 mL×2). The extracts were washed once with a mixture of saturated aq. NaHCO₃ (5 mL) and saturated aq. NaCl (5 mL), dried (MgSO₄), and concentrated.

To a solution of the residue and N,N-dimethylaminopyridine (˜3.5 mg) in pyridine (0.1 mL) was added (R)-(−)-α-methoxy-α-(trifluoromethyl)phenylacetyl chloride at room temperature. After stirring for 1.5 h, the pyridine was evaporated and the residue was dissolved in 0.5 N HCl (10 mL) and ethyl acetate (10 mL). After the separation of the two layers, the organic layer was washed with water (10 mL×1) and saturated aq. NaHCO₃ (10 mL×1), dried (MgSO₄), and concentrated. In the ³¹P NMR of the residue in DMSO-d₆, the desired amide appears at 23.00 ppm whereas the undesired amide comes at 22.79 ppm.

### 2. Preparation of Phosphonic Acid Intermediates:

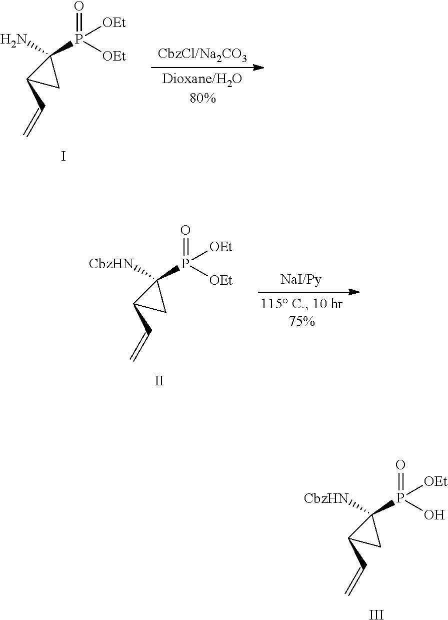

Amine I (9.0 g, 41.1 mmol) was dissolved in 1,4-dioxane (100 mL). A solution of Na₂CO₃ (13.1 g, 123.3 mmol) in H₂O (50 mL) was added to the reaction mixture and stirred for 5 minutes at room temperature. After benzyl chloroformate (8.4 g, 49.3 mmol) was added, the reaction solution was stirred at room temperature overnight. The organic phase was diluted with EtOAc and extracted with H₂O and brine. The organic phase was dried over MgSO₄. Concentration of the filtrate from vacuum filtration removal of the MgSO₄ yielded an oil from which II was isolated by column chromatography (SiO₂, 20% EtOAc in hexane) as a clear oil (11.6 g, 80%). ¹H NMR (300 MHz, CDCl₃) δ 7.33 (s, 5H), 6.05 (dt, J=9.9, 17.1 Hz, 1H), 5.65 (d, J=23.7 Hz, 1H), 5.³¹ (d, J=17.1 Hz, 1H), 5.06 (m, 3H), 4.06 (m, 4H), 2.09 (m, 1H), 1.73 (m, 2H), 1.15 (dt, J=8.1, 26.4 Hz, 6H). ³¹P NMR (121.4 MHz, CDCl₃) δ 23.7

Intermediate II (11.6 g, 32.9 mmol) and NaI (24.5 g, 164.3 mmol) were dissolved in pyridine (110 mL). The reaction solution was heated to 115° C. for 10 hours. After cooling back to room temperature, the reaction solution was concentrated to remove pyridine. H₂O (50 mL) was added to the crude. The aqueous was washed by diethyl ether (2×100 mL). Then the aqueous phase was adjusted to pH=2 by adding 1 M HCl (aq.) Product III (7.5 g, 23.0 mmol) was isolated by extracting with dichloromethane and used for next step without further purification. ¹H NMR (300 MHz, CDCl₃) δ 8.63 (br, 1H), 7.33 (s, 5H), 5.95 (dt, J=9.9, 17.1 Hz, 1H), 5.65 (d, J=23.7 Hz, 1H), 5.³¹ (d, J=17.1 Hz, 1H), 5.06 (m, 3H), 4.06 (m, 2H), 2.09 (m, 1H), 1.73 (m, 2H), 1.23 (dt, J=8.1, 26.4 Hz, 3H)

³¹P NMR (121.4 MHz, CDCl₃) δ 24.6. LC/MS=326 (M⁺+1), 348 (M⁺+Na)

### 3. Synthesis of Intermediate XII:

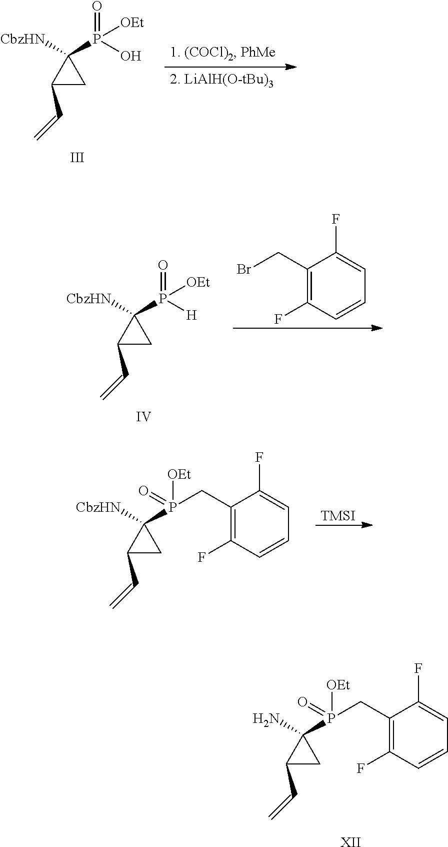

Phosphonic acid intermediate III (1.0 g, 3.1 mmol) was dissolved in toluene (6 mL). This solution was then added dropwise to (COCl)₂ (1.1 mL, 12.4 mmol) and DMF (47 μL, 0.6 mmol) dissolved in 6 mL of toluene at room temperature. After 1 hour of stirring at room temperature, the reaction was concentrated and azeotroped three times with toluene to afford crude IV as an oil.

The resulting dark, viscous residue in THF (20 mL) was stirred at −78° C. as 1.0 M LiAlH(O-tBu)₃ (23.5 mL, 23.5 mmol) was added over 10 minutes. The solution was warmed to r.t. over 30 minutes. The reaction mixture was cooled to 0° C. and quenched with ice cold 1 N HCl (200 mL). The product was extracted with ether (200 mL×2) and the organic fractions were washed with ice cold 1 N HCl (100 mL) and H₂O (100 mL). After the organic fraction was dried (MgSO₄) and concentrated, the residue was purified by combi-flash column chromatography using hexane/ethyl acetate as eluent to obtain IV (1.89 g, 78.3%). ¹H NMR (300 MHz, CDCl₃): δ 8.14 (bs, 1H), 7.35 (s, 5H), 6.22 (s, 1H), 5.89 (m, 2H), 5.39 (bd, J=11.7 Hz, 1H), 5.30 (s, 2H), 5.21-5.104 (m, 3H), 4.13 (m, 2H), 2.16 (m, 1H), 1.72-1.66 (m, 2H), 1.31 (m, 3H).

³¹P (121.4 MHz, CD₃OD): [symbol] 32.311, 29.241

The phosphonous acid IV (327 mg, 1.06 mmol) was suspended in 5 mL of THF and cooled to −40° C. 1N NaN(TMS)₂ (1.27 mL, 1.39 mmol) was added dropwise over 15 minutes followed by 2-(bromomethyl)-1,3-difluorobenzene (176 μl, 1.39 mmol) in 1 mL of THF. The solution stirred from −40° C. to room temperature overnight. The reaction was diluted with EtOAc and quenched with 20 mL of 1N HCl. The organic layer was washed with brine, dried over MgSO₄, filtered and concentrated. The crude material was purified using a CombiFlash Chromatography System using a gradient of 30% EtOAc/Hex to 100% EtOAc to obtain (1-benzyloxycarbonylamino-2-vinyl-cyclopropyl)-(2,6-difluoro-benzyl)-phosphinic acid ethyl ester (147 mg, 33%) as a brown oil. The phosphinate (94.7 mg, 0.22 mmol) was suspended in 1 mL of CH₃CN and cooled to 0° C. Iodotrimethylsilyl (TMSI) (155 μl, 1.08 mmol) was added and the solution was warmed to room temperature. After 45 minutes, the solution was cooled again to 0° C. and triethylamine (1 mL, 7.33 mmol) and 2 mL of MeOH. The solution was warmed to room temperature and stirred for an additional 20 minutes. The solution was concentrated, azeotroped 2× with toluene and put on high vacuum for 30 minutes to provide crude amine, (1-Amino-2-vinyl-cyclopropyl)-(2,6-difluoro-benzyl)-phosphinic acid ethyl ester (Intermediate XII).

### 4. Synthesis of Intermediate XI:

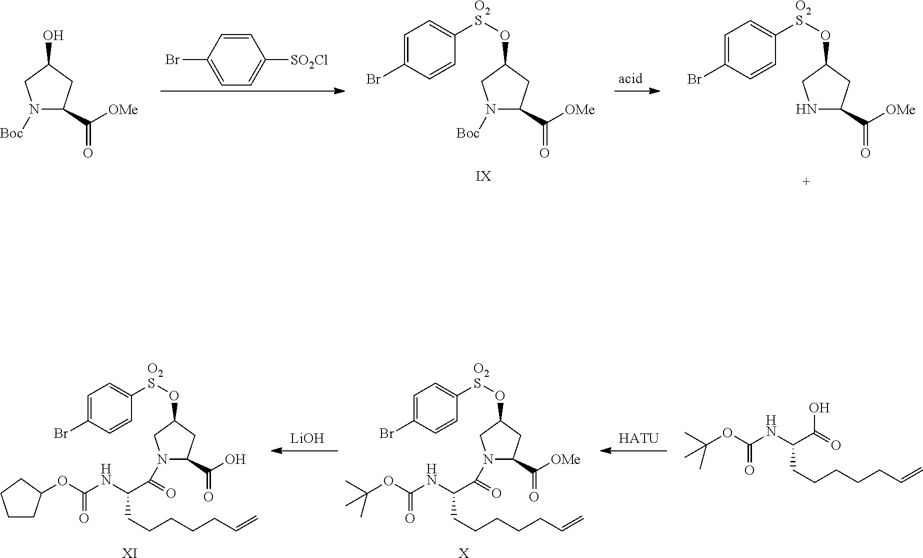

Amine (7.00 g, 28.55 mmol) and DABCO (5.13 g, 45.94 mmol) were dissolved in toluene (30 mL). A toluene (11 mL) solution of brosylchloride (10.22 g, 40.01 mmol) was added. The reaction mixture was stirred at room temperature overnight. The reaction was diluted with EtOAc (210 mL) and 0.5 N HCl (200 mL) was added. The two layers were separated and the aqueous layer was extracted with EtOAc (2×200 mL). The combined organic layers were washed with brine (200 mL), dried with Na₂SO₄, filtered, and concentrated. The crude product was purified by combi-flash to give 12.23 g of intermediate IX in 92% yield.

To a solution of X (12.8 g, 20.7 mmol) in CH₂Cl₂ (50 mL) was added 4 N HCl in 1,4-dioxane (50 mL, 200 mmol). The reaction mixture was stirred at room temperature for 2 h, concentrated, dried under vacuum for 20 minutes, and then dissolved in CH₃CN (50 mL). Saturated NaHCO₃ in H₂O (50 mL) was added and stirred for 5 minutes. Freshly prepared cyclopentylchioroformate in THF (50 mL) was added. The reaction was complete within 1 h. The solvent was removed under reduced pressure and the residue was diluted with EtOAc. The mixture was brought to pH=2 with 1 N HCl and the two layers were separated. The organic layers were washed with brine, dried with Na₂SO₄, filtered, and concentrated to give crude product (3.18 g).

The crude ester (3.18 g, 5.07 mmol) was dissolved in THF (25 mL), H₂O (25 mL), and then MeOH (6 mL) and LiOH (660 mg, 25.4 mmol) was added. The reaction mixture was stirred at room temperature for 1 h and diluted with EtOAc. The reaction mixture was acidified to pH 2 with 1 N HCl and the two layers were separated. The aqueous layer was extracted with EtOAc (2×). The combined organic layers were washed with brine, dried with Na₂SO₄, concentrated and dried under vacuum to give 3.09 g of acid XI.

### 5. Synthesis of Compound 2:

Intermediate XI (17.42 g, 28.30 mmol) was dissolved in THF (136 mL) and cooled to 0° C. To the solution was added N-methylmorpholine (4.7 mL, 42.7 mmol). After 10 min at 0° C., i-butylchloroformate (4.05 mL, 30.96 mmol) was added dropwise. After an additional 1 h, (1-amino-2-vinyl-cyclopropyl)-(2,6-difluoro-benzyl)-phosphinic acid ethyl ester (Intermediate XII, 8.94 g, 29.70 mmol) was slowly added as a solution in THF (20 mL). The suspension was warmed to room temperature and after 2 h it was partitioned between H₂O (400 mL) and ethyl acetate (200 mL). The aqueous layer was extracted with ethyl acetate (200 mL×2) and the combined organic layers were washed with HCl (1N, 225 mL) and H₂O (200 mL). The acid wash and aqueous wash were combined and back-extracted with ethyl acetate (175 mL×2, 100 mL×2). The combined organic layers were washed with brine (400 mL), dried over Na₂SO₄, and concentrated in vacuo providing 25.06 g of diene product in 98.5% crude yield. LCMS (M+1): 898.06. The crude diene product (12.91 g, 14.36 mmol) was dissolved in CH₂Cl₂ (1440 mL) and the solution was degassed for 30 min. The solution was heated to 40° C. and Grubb's G1 catalyst (2.95 g, 3.59 mmol) was added. The reaction was refluxed for 17 h whereupon tris-hydroxymethylphosphine (22.3 g, 18.0 mmol), TEA (50 mL, 35.9 mmol), and H₂O (400 mL) were added and the reaction mixture was heated to reflux for an additional 16 h. The reaction mixture was cooled to room temperature and the two layers were separated. The organic layer was washed with H₂O (400 mL) and brine (300 mL), dried over MgSO4, and concentrated. The crude residue was purified by silica-gel chromatography to afford 8.30 g of macrocyclic olefin product in 66% yield. LCMS (M+1): 870.09.

The macrocyclic olefin (7.34 g, 8.42 mmol) was dissolved in ethyl acetate (105 mL) and rhodium on alumina (5% wt, 2.945 g, 0.40 wt %) was added. The system was evacuated and flushed with H₂ (1 atm, 3×). To the system, after 3 h, was added more rhodium on alumina (5% wt, 842 mg, 0.10 wt %) and evacuated and flushed with H₂ (1 atm, 3×). After an additional 1 h the suspension was filtered and concentrated in vacuo providing 6.49 g of reduced macrocycle in 88% crude yield. LCMS (M+1): 872.04.

The brosylate macrocycle (6.49 g, 7.67 mmol) was dissolved in N-methylpyrrolidinone (25.0 mL) and 8-chloro-2-(2-isopropylamino-thiazol-4-yl)-7-methoxy-quinolin-4-ol (2.564 g, 7.33 mmol) followed by Cs₂CO₃ (4.40 g, 13.50 mmol) were added. The mixture was heated to 65° C. for 6 h then diluted with ethylacetate (200 mL) and washed with LiCl (5%, 250 mL). The aqueous layer was extracted with ethylacetate (100 mL×2) and the combined organic layers were washed with brine (150 mL), dried over Na₂SO₄/MgSO₄, and concentrated in vacuo. The crude residue was purified via silica-gel chromatography (ethylaceate-methanol) affording 4.39 g of aminothiazole product in 58% yield. LCMS (M+1): 985.28.

Phosphinate ester (23.7 g, 24.05 mmol) was dissolved in CH₃CN (240 mL) and cooled to 0° C. Iodotrimethylsilane (17.4 mL, 122.3 mmol) was added at a fast drop-wise pace followed by, after 10 min, 2,6-lutidine (17.0 mL, 146.4 mmol). The reaction mixture was slowly warmed to room temperature and stirred for 1 h then cooled back down to 0° C. and 2,6-lutidine (11.1 mL, 95.6 mmol) followed by MeOH (24 mL) were added. The solution was concentrated in vacuo and the crude residue was purified by HPLC to afford 12.68 g of Compound 2 in 55% yield.

¹H NMR (300 MHz, CDCl₃) δ 8.35 (d, J=9.3 Hz, 1H), 8.28 (s, 1H), 7.85 (s, 1H), 7.64 (d, J=9.6 Hz, 1H), 7.35-7.22 (m, 1H), 7.02-6.89 (m, 2H), 5.85 (bs, 1H), 4.82-4.71 (m, 2H), 4.33 (bs, 1H), 4.28-3.99 (m, 3H), 4.16 (s, 3H), 3.57-3.28 (m, 2H), 2.90-2.78m, 1H), 2.63-2.50 (m, 1H), 2.08-1.91 (m, 1H), 1.91-170 (m, 2H), 1.70-1.13 (m, 22H), 1.37 (d, J=6.9 Hz, 6H); 31P NMR (121.4 MHz, CD₃OD) δ 42.4; LCMS (M+1): 957.35. g.

### Example 3

### Preparation of Compound 3

### 1. Preparation of Tripeptide Intermediates:

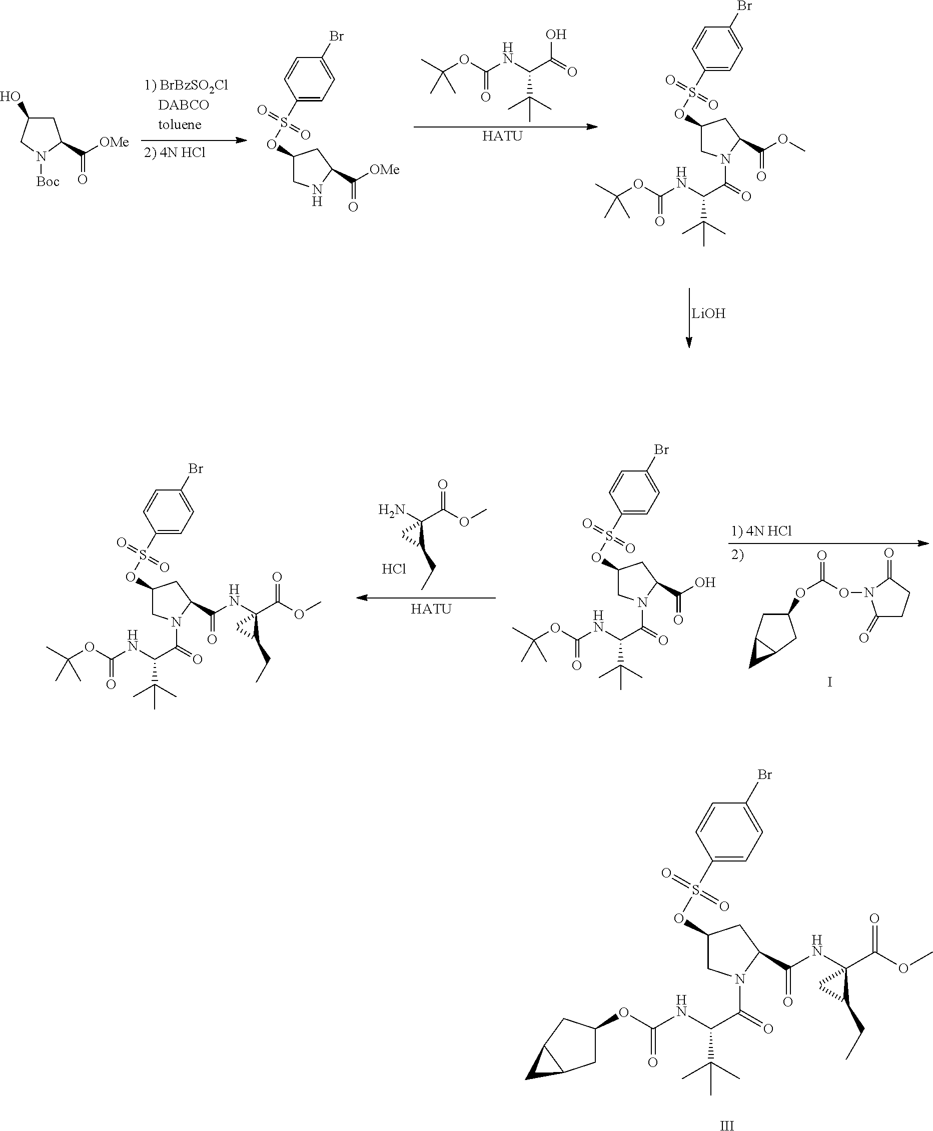

Step 1: N-t-Boc-cis-4-Hydroxy-L-Proline methyl ester (100.0 g, 407.7 mmol) and DABCO (1.5 eq, 68.6 g, 611.6 mmol) were dissolved in anhydrous toluene (200 mL) in a 2 L three necked round bottom flask with a mechanical stirrer and an addition funnel. After cooling the solution to 0° C. under N₂, a solution of 4-Bromo-benzenesulfonyl chloride (1.3 eq, 135.6 g, 530.0 mmol) in 300 mL of toluene was added through addition funnel over 60 minutes. The reaction mixture was stirred and warmed to room temperature overnight (16 hours). The mixture was slowly poured into 2 L 1M Na₂CO₃ (aq.), and the product was extracted with EtOAc (2 L). After the organic phase was washed by 0.5 N HCl (2 L), H₂O (1 L), and brine (1 L), it was dried (MgSO₄), concentrated to give 195.45 g of a yellow oily brosylate product.

To a solution of the above brosylate (407.7 mmol) in dichloromethane (300 mL) was slowly added 4.0 M HCl in dioxane (500 mL, 5 eq) and the resulting solution was allowed to stir at room temperature for 2 hours. After ether (500 mL) was added to the reaction mixture, the mixture was stirred for 15 min and the white precipitate was collected by filtration. The solid was washed with ether and hexane and then dried under vacuum overnight to obtain 153.0 g of the HCl amine salt, 381.8 mmol, in 94% yield for two steps.

Step 2: To a solution of Boc-tert-butyl-glycine (97.0 g, 420.0 mmol) in DMF (200 mL) and DCM (200 mL) were added HATU (217.76 g, 572.7 mmol) and Hunig's base (126 mL, 1145.4 mmol) at room temperature. After the mixture was stirred for 20 min at room temperature, a solution of the previous HCl salt (153.0 g, 381.8 mmol) and Hunig's base (126 mL, 1145.4 mmol) in DMF (200 mL) and dichloromethane (200 mL) was added to the above acid mixture in one portion. The reaction mixture was stirred at room temperature for 3 h, with monitoring by LCMS. The reaction mixture was concentrated to remove dichloromethane under reduced pressure and the white solid that formed was filtered off. The remaining DMF solution was diluted with ethyl acetate (1 L), washed successively with 3% LiCl (aq) (3×650 mL), sat'd NH₄Cl (2×500 mL), 0.5N HCl (aq) (2×600 mL), brine (500 mL), sat'd NaHCO₃ (3×500 mL), and brine (500 mL). The resulting organic fraction was dried (MgSO₄) and concentrated to afford crude tripeptide (111 g).

Step 3: To a solution of the methyl ester (120 g, 207.8 mmol) in THF (300 mL), MeOH (75 mL) was added a solution of LiOH (26.18 g, 623.4 mmol) in H₂O (150 mL). The solution was allowed to stir at room temperature for 4 hours. The mixture was cooled in an ice-bath while acidifying with 3N HCl to pH about 5.5, stirred for 10 min, and the resulting white solids were collected by filtration. The solids were washed with more water, ether and hexane. The solids were dried under vacuum at 40° C. overnight to give 95.78 g (82%) of the acid.

Step 4: To a solution of the carboxylic acid (81.4 g, 144.27 mmol) in DMF (200 mL) and dichloromethane (200 mL) was added HATU (82.3 g, 216.4 mmol) and Hunig's base (47.5 mL, 432.8 mmol) at room temperature. After the mixture was stirred for 20 min at room temperature, a solution of amine (158.7 mmol) and Hunig's base (47.5 mL, 1145.4 mmol) in DMF (200 mL) and dichloromethane (200 mL) was added to the above acid mixture in one portion. The reaction mixture was stirred at room temperature for 3 h and monitored by LCMS. After the mixture was concentrated under reduced pressure to remove dichloromethane, the white solids that formed were filtered off. The remaining DMF solution was diluted with ethyl acetate (600 mL) and successively washed with 3% LiCl (aq) (2×550 mL), sat'd NH₄Cl (500 mL), 1N HCl (aq) (500 mL), sat'd NaHCO₃ (500 mL), and brine (300 mL). The resulting organic fraction was dried (Na₂SO₄) and concentrated to afford crude tripeptide (111 g).

Step 5: The crude tripeptide was dissolved in 4N HCl in dioxane (300 mL) at room temperature and stirred for 2 h. It was then concentrated under vacuum, and co-evaporated with dichloromethane (2×200 mL) to dryness. The residue was dissolved in EtOAc (600 mL) and sat'd aq. NaHCO₃ (1 L). It was stirred vigorously. After 10 min, carbonic acid bicyclo[3.1.0]hex-3-yl ester 2,5-dioxo-pyrrolidin-1-yl ester (intermediate I, 41.4 g, 173.1 mmol) was added in one portion. After the resulting mixture was stirred for another 30 min, the organic layer was collected and washed with brine (500 mL), dried (Na₂SO₄), and concentrated. The crude product was purified by flash chromatography on silica gel with ethyl acetate/hexane to afford 94.44 g (92%) of the tripeptide intermediate III.

### 2. Preparation of Quinoline Intermediate IV:

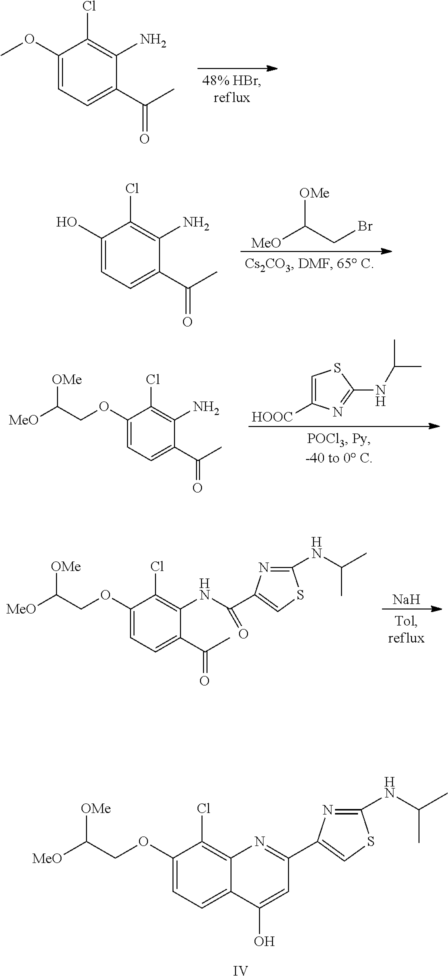

Step 1: 1-(2-Amino-3-chloro-4-hydroxy-phenyl)-ethanone (70.7 g, 354 mmol) was stirred in 48% aq. HBr (500 mL) at 110° C. for 72 h. After the mixture was cooled to 0° C. with stirring, the solids were filtered and washed with water. The resulting solids were triturated with a saturated NaHCO₃ solution (˜350 mL), filtered, washed with water, and dried under vacuum to give ˜40 g (61%) of crude product as a dark brown solids. LC/MS=186 (M⁺+1).

Step 2: 1-(2-Amino-3-chloro-4-hydroxy-phenyl)-ethanone (40 g, 215 mmol) was dissolved in DMF (360 ml). Cesium carbonate (140 g, 430 mmol) was added, followed by bromoacetaldehyde dimethyl acetal (54.5 g, 323 mmol). The mixture was then vigorously stirred at 65° C. for 24 h. Upon cooling to room temperature, EtOAc (1 L) and H₂O (1 L) were added to the mixture. The organic layer was extracted with EtOAc (1×400 ml). The combined organic layer was washed with aqueous 3% LiCl solution (2×1 L), brine, dried (Na₂SO₄) and concentrated in vacuo. The residue was purified by silica gel chromatography to give the desired product as a white solid (39 g, 67%).

Step 3: To a mixture of 1-[2-Amino-3-chloro-4-(2,2-dimethoxy-ethoxy)-phenyl]-ethanone (13 g, 47.5 mmol) and isopropylaminothiazole-4-carboxylic acid hydrobromide (12.64 g, 47.5 mmol) in pyridine (150 ml) was slowly added phosphorus oxychloride (9.47 g, 61.8 mmol) at −40° C. The mixture was then stirred at 0° C. for 4 h. Upon completion of the reaction, H₂O (30 ml) was added dropwise to the mixture. The mixture was then stirred at 0° C. for another 15 min. The mixture was concentrated in vacuo. The residue was diluted with EtOAc, washed with a sat. NaHCO₃ aqueous solution. The organic layer was dried (Na₂SO₄) and concentrated in vacuo. The residue was dissolved in CH₂Cl₂, hexanes was added slowly to the solution, and a yellow solid started to crash out. More hexanes were added until not much product was left in the mother liquid (18 g, 85%).

Step 4: 2-Isopropylamino-thiazole-4-carboxylic acid [6-acetyl-2-chloro-3-(2,2-dimethoxy-ethoxy)-phenyl]-amide (18 g, 40.7 mmol) was suspended in toluene (400 ml). NaH (2.4 g, 61 mmol) was added to the vigorously stirred mixture while monitoring H₂ evolution. The mixture became a clear solution during heating to reflux. The reaction was complete after refluxing for 3 h. The mixture was cooled to room temperature. A solution of AcOH (69.2 mmol) in H₂O (3 vol) was added to the mixture. After vigorous agitation for 1 h at 0° C., the solids were collected by filtration, rinsed forward with H₂O. The wet cake was dried under high vacuum to a constant weight to provide intermediate IV (15 g, 86%).

### 3. Preparation of Compound 3:

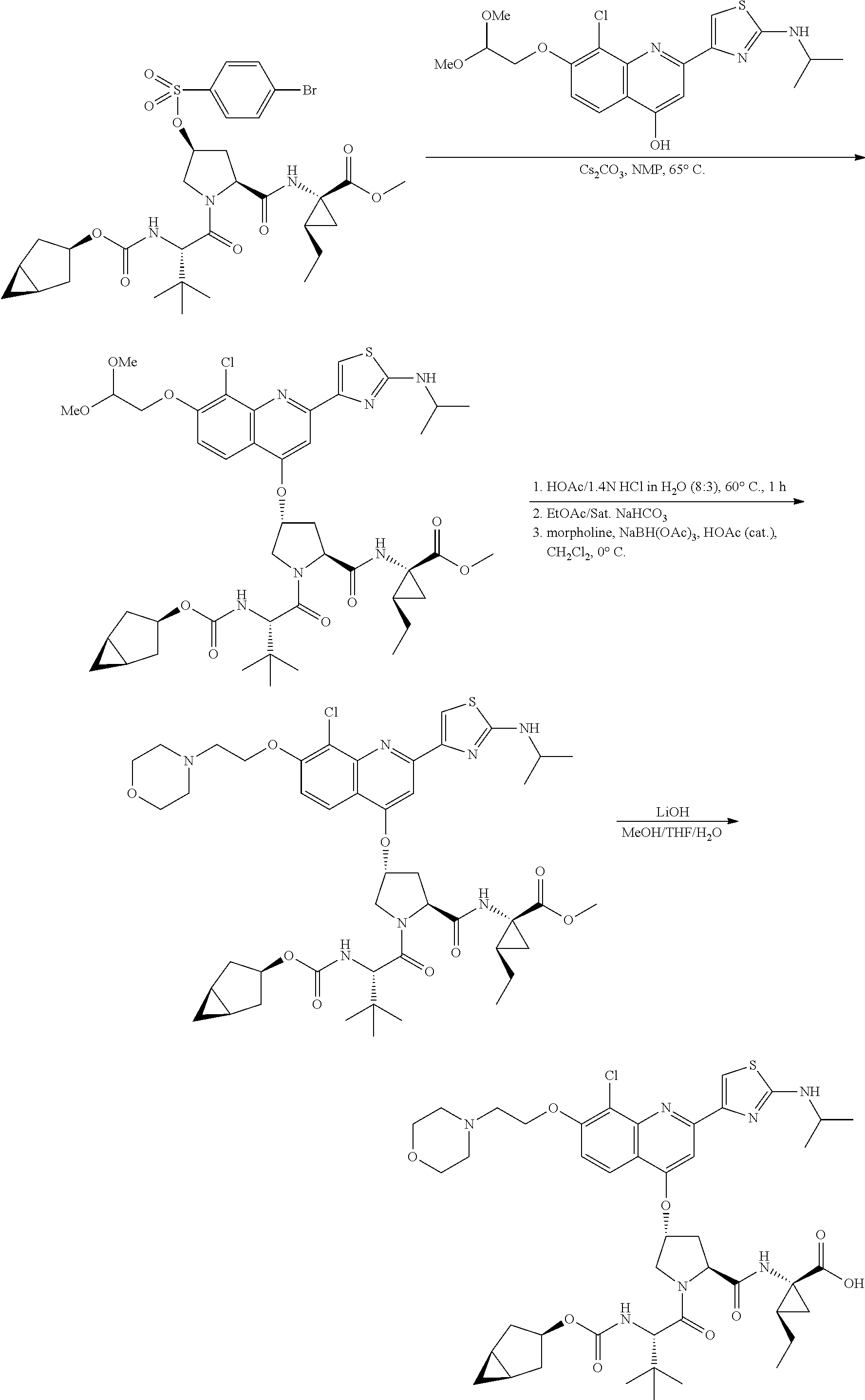

### Step 1

To a mixture of depicted brosylate intermediate (15 g, 35 mmol) and depicted phenol intermediate (27.5 g, 38.5 mmol) in NMP (200 ml) was added cesium carbonate (25.1 g, 77 mmol). The mixture was stirred at 65° C. for 5 h. The reaction was cooled to room temperature and EtOAc (600 ml) and an aqueous solution of 3% LiCl (600 ml) were added to the mixture. The organic layer was washed with aqueous 3% LiCl (1×600 ml), brine, dried (Na₂SO₄) and concentrated in vacuo. The residue was purified by silica gel chromatography to give the desired methyl ester as a yellow solid (23.6 g, 75%). LC/MS=900.13(M++1).

### Step 2

Methyl ester (23.6 g, 26 mmol) was dissolved in glacial acetic acid (200 ml), 1.4 N HCl in H₂O (75 ml) was added to the solution. The mixture was stirred at 60° C. for 1 h. Upon completion of the reaction, the mixture was concentrated to remove the solvents, co-evaporated with toluene (×2) to remove residual acetic acid. The residue was then dissolved in EtOAc (500 ml) and saturated NaHCO₃ aqueous solution (enough to neutralize the mixture) while monitoring CO₂ evolution. The organic layer was washed with brine, dried (Na₂SO₄) and concentrated in vacuo. The residue was further dried under high vacuum for 1 h and used as is for the next step. The crude product was dissolved in CH₂Cl₂ (360 ml), morpholine (3.4 g, 39 mmol) and sodium triacetoxyborohydride (7.2 g, 34 mmol) were added to the mixture at 0° C. Then glacial acetic acid (0.47 g, 7.8 mmol) was added dropwise to the mixture. The reaction was complete in 10 min at 0° C. Saturated NaHCO₃ aqueous solution was added to quench the reaction. After stirring for another 20 min, the organic layer was washed with brine, dried (Na₂SO₄) and concentrated in vacuo. The residue was purified by silica gel chromatography to give the desired amine product as a yellow solid (12 g, 50%). LC/MS=924.63(M++1).

### Step 3

Amine (12 g, 13 mmol) was dissolved in THF (200 ml), LiOH (11 g, 260 mmol) in H₂O (200 ml) was added, followed by MeOH (200 ml). The mixture was kept stirring at room temperature for 20 h. Upon completion of the reaction, 4 N HCl in H₂O was added to adjust pH to 7 at 0° C. The mixture was extracted with EtOAc (2×400 ml). The combined organic layer was washed with brine, dried (Na₂SO₄) and concentrated in vacuo to give Compound 3 as a yellow solid (11 g, 93%). LC/MS=911.52(M++1).

¹H NMR (300 MHz, CD₃OD) δ 7.95 (d, 1H), 7.90 (s, 1H), 7.48 (s, 1H), 7.31 (d, 1H), 5.42 (s, 1H), 4.37 (dd, 1H), 4.20 (m, 2H), 3.83-3.56 (m, 7H), 3.50 (m, 2H), 3.39 (m, 2H), 2.45 (m, 1H), 2.27(m, 1H), 1.62 (m, 2H), 1.50 (m, 1H), 1.33 (m, 2H), 1.18 (m, 1H), 1.05 (m, 8H), 0.90 (m, 3H), 0.76 (m, 11H), 0.14-0.04 (m, 2H).

### Example 4

### Preparation of Compound 4

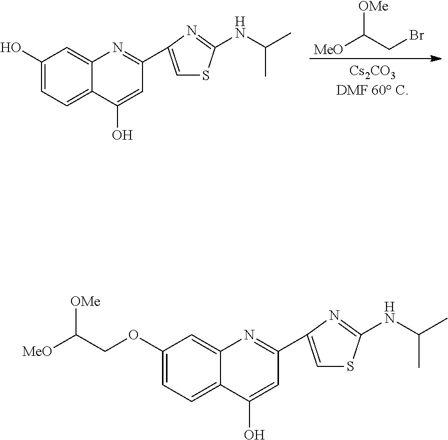

### Step 1:

Bis-phenol (7 g, 23.4 mmol) was dissolved in DMF (50 ml), cesium carbonate (15.25 g, 46.8 mmol) was added to the mixture, followed by bromoacetaldehyde dimethyl acetal (4.13 mL, 35.1 mmol). The mixture was then vigorously stirred at 65° C. and monitored by HPLC and LC/MS. Another 0.5 eq of bromoacetaldehyde dimethyl acetal and 1 eq of cesium carbonate were added. After 18 hrs LC/MS indicated no starting material remained, but lots of bis-alkylated by-product formed. The reaction was cooled to room temperature, and diluted with EtOAc. The mixture was washed with aqueous 3% LiCl solution, brine, dried (Na₂SO₄) and concentrated in vacuo. The residue was purified by silica gel chromatography with MeOH/EtOAc to give the desired product (1.72 g, 19%). LC/MS=390 (M⁺+1).

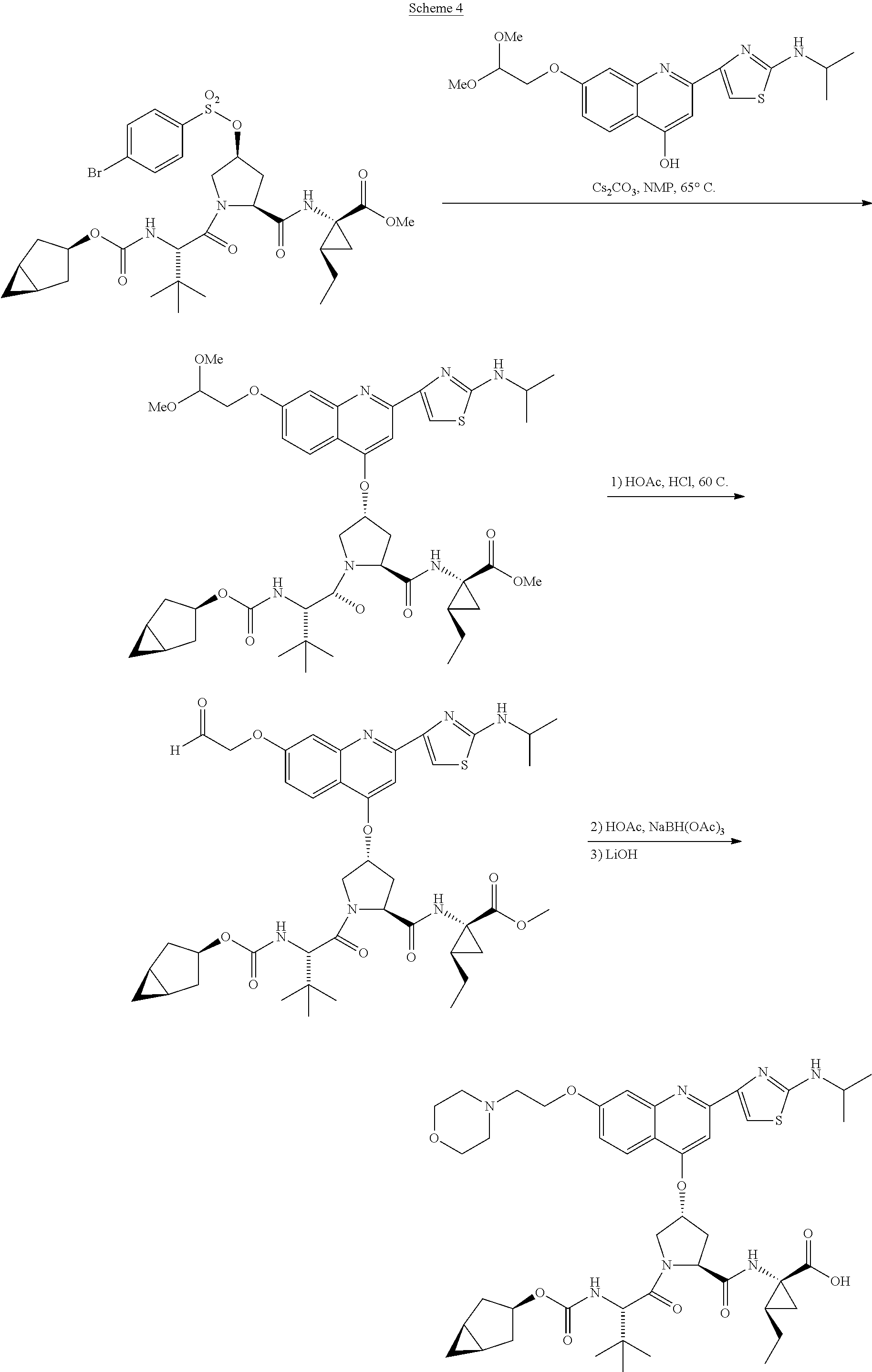

### Step 2:

To a mixture of tripeptide (1.46 g, 3.75 mmol) and cesium carbonate (1.58 g, 4.88 mmol) in NMP (18.5 ml) at room temperature was added quinoline (2.94 g, 4.12 mmol) in one portion. The mixture was stirred at 65° C. for 3 h. The reaction was cooled to room temperature, and EtOAc (100 ml) was added to the mixture. The mixture was washed with aqueous 3% LiCl (1×100 ml), brine, dried (Na₂SO₄) and concentrated in vacuo. The residue was purified by silica gel chromatography with EtOAc/Hexane to give the desired product as light brown solid (2.07 g, 64%). LC/MS=837 (M⁺+18).

### Step 3:

To a solution of the acetal (1.24 g, 1.43 mmol) in glacial acetic acid (16 mL) was added 1.4 N HCl in H₂O (6 mL). The mixture was stirred at 60° C. for 1.5 h. Upon completion of the reaction, the mixture was concentrated to remove the solvents, coevaporated with toluene (×2) to remove residual acetic acid. After the residue was then dissolved in EtOAc (100 mL) and sat. NaHCO₃ (100 mL), the organic layer was separated, washed with brine, dried (Na₂SO₄) and concentrated in vacuo. The residue was further dried under high vacuum for 1 h to obtain the aldehyde (1.16 g), and used as is for the next step.

### Step 4:

The crude aldehyde was dissolved in CH₂Cl₂ (16 ml), and then morpholine (164 μl, 1.89 mmol) and sodium triacetoxyborohydride (462 mg, 2.18 mmol) were added to the mixture at 0° C. Glacial acetic acid (25 μl, 7.8 mmol) was then added dropwise to the mixture. The reaction was complete in 10 min at 0° C. Sat. aqueous NaHCO₃ solution was added to quench the reaction. After stirring for another 20 min, the organic layer was washed with brine, dried (Na₂SO₄) and concentrated in vacuo. The crude product was clean enough (by LC/MS) to use as is. LC/MS=890 (M⁺+1).

This crude product was dissolved in THF (60 ml), and then LiOH (1200 mg, 28.6 mmol) in H₂O (20 ml) was added, followed by MeOH (4 ml). The mixture was kept stirring at room temperature for 20 h. Upon completion of the reaction, TFA was added at 0° C., to adjust the pH to 4. The mixture was extracted with EtOAc (2×200 ml). The combined organic layer was washed with brine, dried (Na₂SO₄) and concentrated in vacuo to give the crude product. The crude product was purified by prep-HPLC to give Compound 4 as a yellow solid (1.086 g, 73%). LC/MS=876 (M⁺+1). ¹H NMR (300 MHz, CD₃OD): δ 7.94 (d, 1H), 7.40 (s, 1H), 7.44 (d, 1H), 7.39 (s, 1H), 7.04-7.01 (m, 1H), 5.39 (m, 1H), 4.32-4.20 (m, 5H), 3.80-3.68 (m, 4H), 3.59 (bs, 3H), 3.40 (m, 2H), 3.35-3.24 (m, 4H), 3.93-3.92 (m, 2H), 2.40-2.19 (m, 2H), 1.65-1.47 (m, 2H), 1.33-1.25 (m, 3H), 1.16-1.11 (m, 1H), 1.05-1.01 (m, 1H), 0.96 (s, 3H), 0.95 (s, 3H), 0.86-0.79 (m, 3H), 0.65 (s, 9H), 0.57 (m, 2H).

### Example 5

### Preparation of Compound 5

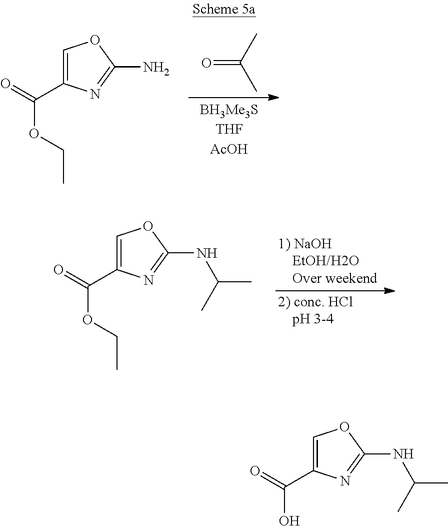

### Step 1:

The mixture of 2-amino-oxazole-4-carboxylic acid ethyl ester (500 mg, 3.2 mmol) and acetone (2.35 mL, 32 mmol) in THF (6 mL) was stirred at room temperature. Borane (BH₃.Sme₂) (10M in THF, 0.64 mL, 6.4 mmol) was added slowly via syringe to control the exotherm and bubbling. Next, AcOH (0.362 mL, 6.4 mmol) was added in the same manner. (Another 2 eq of borane and AcOH were added 18 h later) The mixture was stirred under a nitrogen atmosphere and monitored by LC/MS. After 3 days at room temperature, the reaction still had some SM left. It was concentrated in vacuo. The resulting residue was dissolved in EtOAc (100 mL), washed with saturated NH₄Cl solution, 0.1 M NH₄OH and brine. The organic phase was dried (Na₂SO₄) and concentrated in vacuo. The crude product was purified by flash chromatography on silica gel, eluting with EtOAc/Hexanes to give the desired product (0.40 g, 64%). LC/MS=199 (M⁺+1).

### Step 2:

To the mixture of ester obtained above (2.5, 10.86 mmol) in EtOH (42 mL) and water (28 mL) was added NaOH (3.1 g, 77.4 mmol). The mixture was stirred at room temperature for 16 h. It was monitored by TLC. After the mixture was done, it was cooled in an ice-bath and acidified by adding conc. HCl to adjust the pH to 3. The mixture was then concentrated in vacuo to remove ethanol. The remaining was extracted with CH₂Cl₂ (3×200 mL). The organic phases were combined, dried (MgSO₄) and concentrated to give the desired product (1.86 g, 87%). LC/MS=171 (M⁺+1).

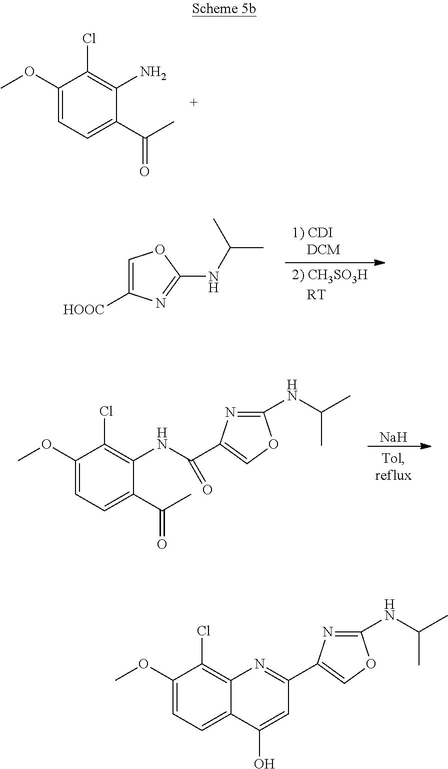

### Step 3:

To acid (1.86 g, 10.94 mmol) in DCM (10 ml) was added CU (1.774 g, 10.94 mmol). The mixture was then stirred at room temperature for 2 h. Aniline (1.446 g, 8.75 mmol) was added followed by CH₃SO₃H (2.13 mL, 32.82 mmol). The reaction was stirred for 18 h at room temperature. Upon completion of the reaction, it was diluted with DCM (100 mL) and washed with 1N HCl (2×100 mL). To this organic phase, was added K₂CO₃ (3.02 g, 21.88 mmol) and stirred for 2 h at room temperature. Solids were removed by filtration, and the filtrate was concentrated in vacuo. The residue was purified by flash chromatography on silica gel, eluted with EtOAc/Hexane to give the desired product (863.4 mg, 22%). LC/MS=382 (M⁺+1).

### Step 4:

The methyl ketone obtained above (863.4 mg, 2.45 mmol) was suspended in toluene (20 ml). NaH (147.3 mg, 3.68 mmol) was added to the vigorously stirred mixture while monitoring H₂ evolution. The reaction was refluxed (110° C.) for 3 h. The mixture was not a clear solution. LC/MS showed still about ⅓ of starting material left. After cooling, about 80 mg of NaH was carefully added, followed by 20 mL of THF to help the solubility. The mixture was heated for another 2 h and the reaction almost reached completion. After cooling to room temperature, it was quenched by the addition of conc. HCl to adjust the pH to about 2-3. The slurry was stirred for 1 h at room temperature. 10 mL of CH₃CN was added, followed by 5 mL H₂O, and then 20 mL of ether. The mixture was stirred for another ½ h, and then the solids were collected by filtration and washed with ether and hexane. The wet cake was dried under high vacuum to a constant weight (390 mg of HCl salt, 840 mg, 100%). LC/MS=334 (M⁺+1).

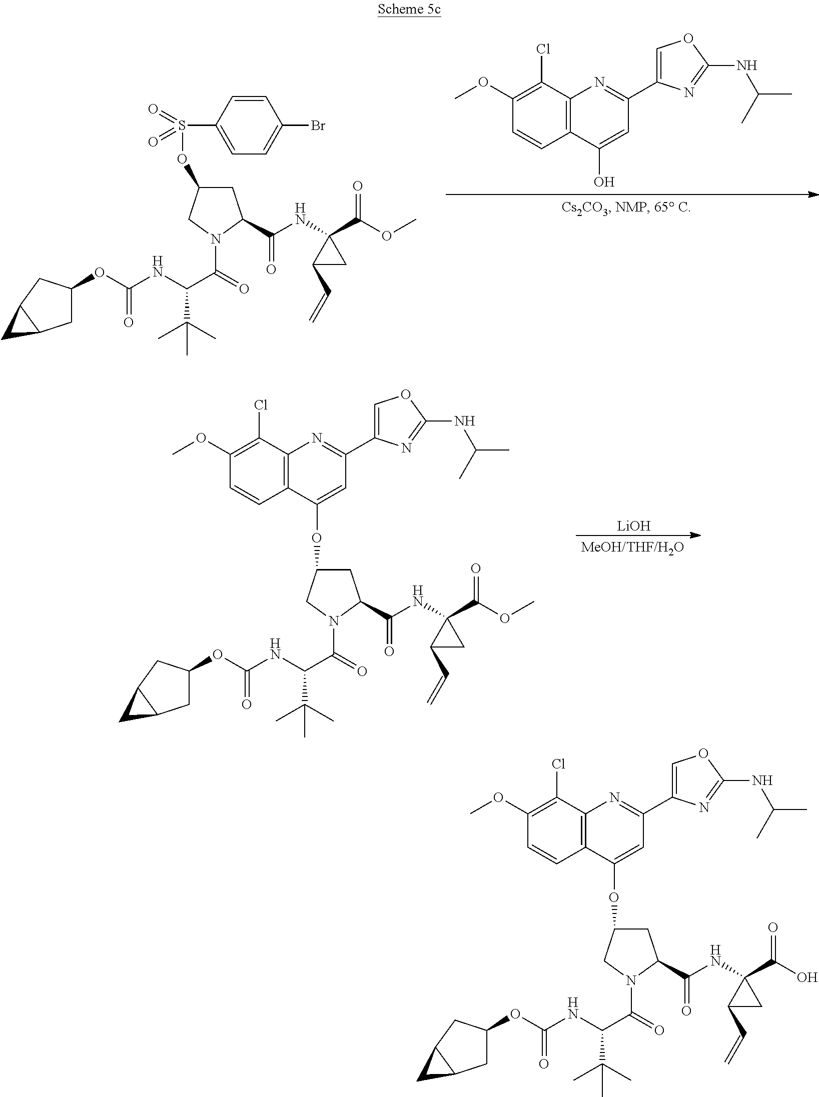

### Step 5:

Using the same procedure described herein, Compound 5 was obtained after prep HPLC purification as a yellow solid (30 mg). LC/MS=794 (M⁺+1). ¹H NMR (300 MHz, CD₃OD): δ 8.74 (s, 1H), 8.54 (s, 1H), 8.25 (d, 1H), 7.59 (m, 2H), 5.90-5.80 (m, 1H), 5.65 (bs, 1H), 5.31-5.09 (dd, 2H). 4.73 (t, 1H), 4.54 (m, 1H), 4.14 (s, 3H), 4.11-3.99 (m, 5H), 2.81-2.60 (m, 2H), 2.2 (m, 1H), 2.00-1.60 (m, 4H), 1.50-1.40 (m, 2H), 1.35 (s, 3H), 1.33 (s, 3H), 1.20 (m, 2H), 1.02 (s, 9H), 0.34 (m, 2H).

### Example 6

### Preparation of Compound 6

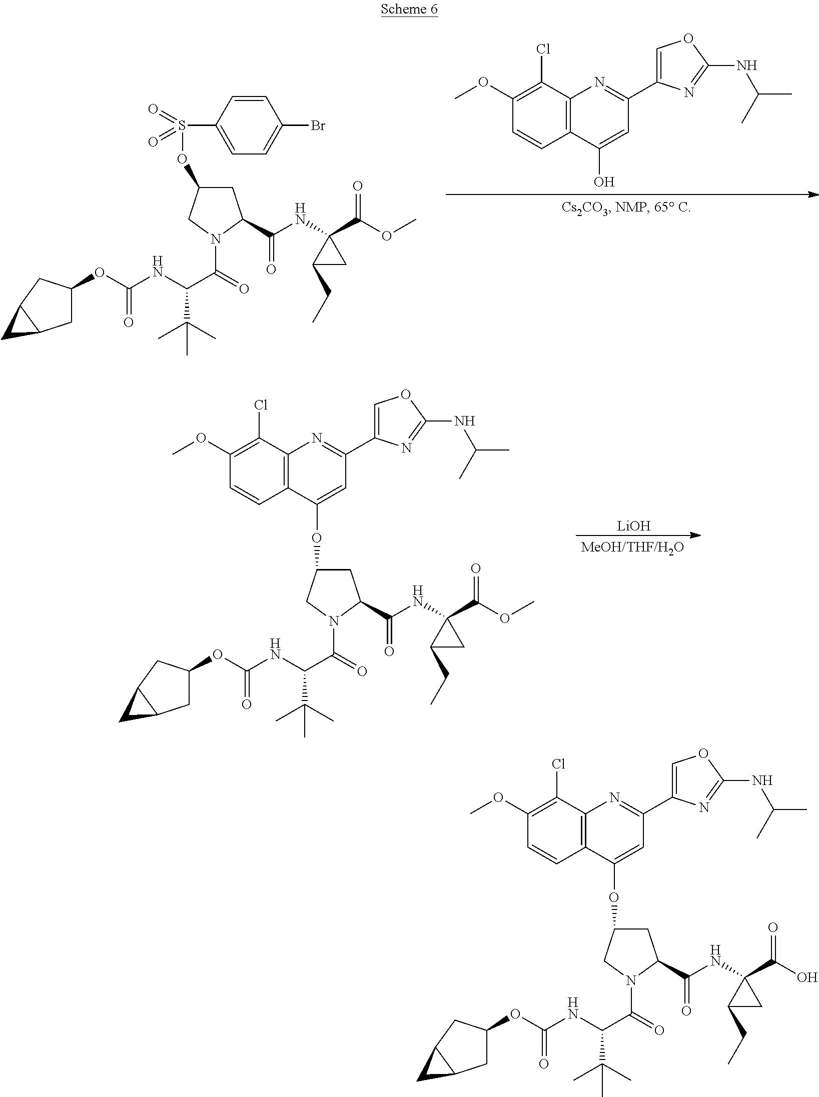

Using the same procedure herein, Compound 6 was obtained after prep HPLC purification as a yellow solid. LC/MS=796 (M⁺+1). ¹H NMR (300 MHz, CD₃OD): δ 8.64 (s, 1H),8.60 (s, 1H), 8.26 (d, 1H), 7.61 (m, 2H), 5.67 (bs, 1H), 4.73 (t, 1H), 4.53 (m, 1H), 4.15 (s, 3H), 4.12 (m, 5H), 2.81-2.60 (m, 2H), 2.2 (m, 1H), 2.00-1.40 (m, 6H), 1.36 (s, 3H), 1.34 (s, 3H), 1.23 (m, 2H), 1.02 (s, 9H), 0.34 (m, 2H).

### Example 7

### Preparation of Compound 7

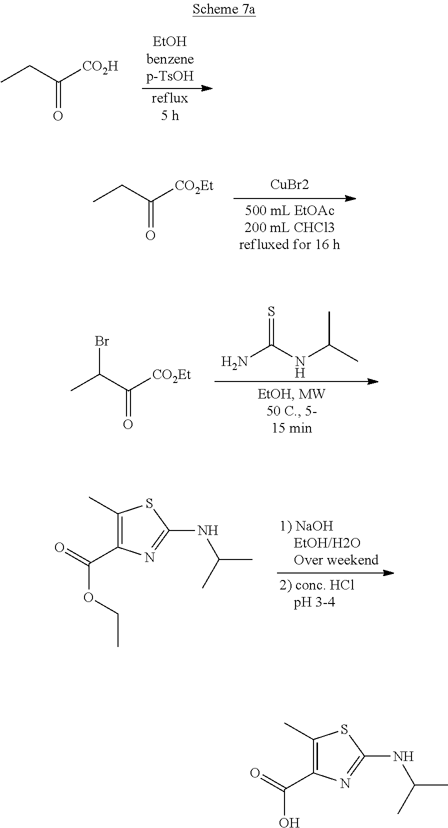

### Step 1:

The mixture of 2-oxo-butyric acid (15 g, 147 mmol), p-TsOH (300 mg) in benzene (60 mL) and EtOH (125 mL) was stirred at 90° C. (reflux) for 5 h. After the mixture was cooled to room temperature, it was concentrated in vacuo (water bath t<20° C.). The resulting residue was dissolved in EtOAc (200 mL), washed with a saturated NaHCO₃ solution and brine. The organic phase was dried (Na₂SO₄) and concentrated in vacuo (water bath below 20° C.) to give the desired product (12.2 g, 64%). ¹H NMR (300 MHz, CDCl₃): δ 4.30 (q, 2H), 2.85 (q, 2H), 1.35 (t, 3H), 1.11 (t, 3H).

### Step 2:

To a suspension of CuBr₂ (32 g, 147.1 mmol) in EtOAc (500 mL) was added the ester (6.2 g, 47.7 mmol) in CHCl₃ (200 mL). The mixture was stirred at 90° C. (reflux) for 16 h. It was monitored by TLC (EtOAc:Hexane=1:4, R_{f}=0.5, R_{f}=0.4). After the mixture was cooled to room temperature, it was filtered through a bed of silica gel eluting with 200 mL of a 1:1 EtOAc:Hexane solution. The filtrate was concentrated in vacuo (water bath t<20° C.) to give the desired product (10.75 g, 108%). No mass can be detected by LC/MS. ¹H NMR (300 MHz, CDCl₃): δ 5.17 (q, 1H), 4.38 (q, 2H), 1.81 (t, 3H), 1.38 (t, 3H).

### Step 3:

The mixture of the bromide (1.672 g, 8 mmol) and isopropyl-thiourea (0.944 g, 8 mmol) in 12 mL of EtOH was microwaved at 50° C. for 15 min. After the mixture was cooled to room temperature, it was concentrated in vacuo. The residue was purified by silica gel flash chromatography, eluting with EtOAc/Hexane to give the desired product. LC/MS=229.9 (M⁺+1).

### Step 4:

To the mixture of the ester (1.7 g, 7.45 mmol) in EtOH (12 mL) and water (8 mL) was added NaOH (1.8 g, 44.7 mmol). The mixture was stirred at room temperature for 16 h. The reaction was monitored by TLC. After the reaction was done, it was cooled in an ice-bath and acidified with conc. HCl to adjust the pH to 3. The mixture was then concentrated in vacuo to remove ethanol. The remaining slurry was extracted with CH₂Cl₂ (3×200 mL). The organic phases were combined, dried (MgSO₄) and concentrated to give the desired acid product (1.2 g, 80%).

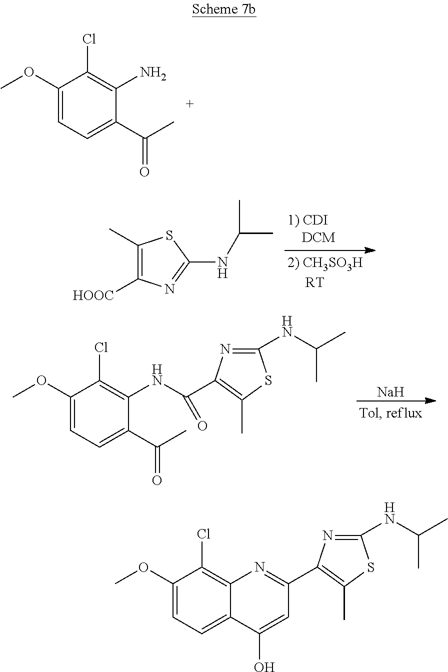

### Step 5:

To the acid (1.2 g, 5.99 mmol) in DCM (10 ml) was added CDI (972 mg, 5.99 mmol. The mixture was then stirred at room temperature for 2 h. Aniline (792 mg, 4.89 mmol) was added followed by CH₃SO₃H (1.17 mL, 18 mmol). The reaction was stirred for 18 h at room temperature. Upon completion of the reaction, it was diluted with DCM (100 mL) and washed with 1N HCl (2×100 mL). To the organic phase, was added K₂CO₃ (1.66 g, 12 mmol) and this mixture was stirred for 2 h at room temperature. Solids were removed by filtration and the filtrate was concentrated in vacuo. The residue was purified by silica gel flash chromatography, eluting with EtOAc/Hexane to give the desired amide product (1.46 g, 70%). LC/MS=382 (M⁺+1).

### Step 6:

The amide compound (1.46 g, 3.82 mmol) was suspended in toluene (30 ml). NaH (0.23 g, 5.73 mmol) was added to the vigorously stirred mixture while monitoring H₂ evolution. The mixture became a clear solution during heating to reflux. The reaction was complete after refluxing for 3 h. The reaction was cooled to room temperature, quenched with IPA (5 mL), and then heptane (30 mL) was added. The slurry was stirred for 1 h at room temperature. The solids that formed were collected by filtration and washed with ether. The collected solids were dissolved in AcCN/H₂O (2:1) and then acidified with 3N HCl. The resulting slurry was stirred for 1 h, and the solids were again collected by filtration. The wet cake was dried under high vacuum to a constant weight (390 mg of HCl salt, 1.07 mmol, 28%). LC/MS=363 (M⁺+1).

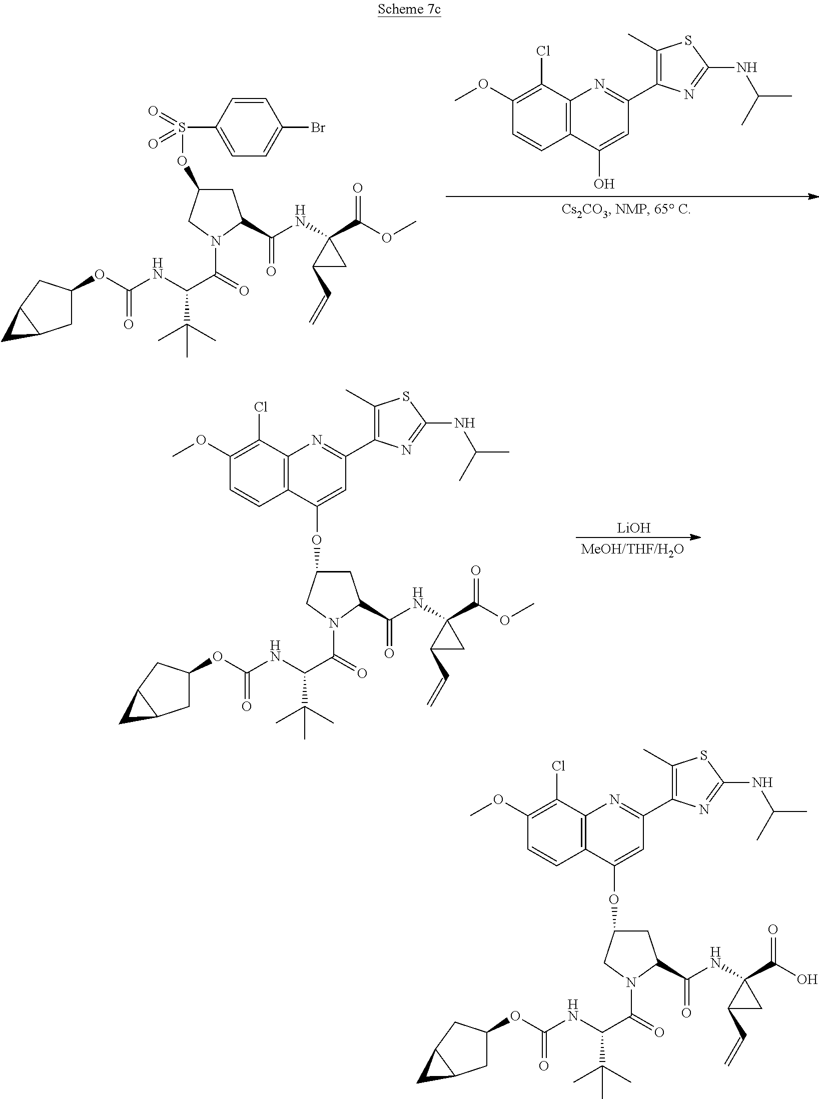

### Step 7:

To a mixture of quinoline (0.39 g, 1.07 mmol) and brosylate (692 mg, 0.974 mmol) in NMP (10 ml) was added cesium carbonate (696 mg, 2.14 mmol). The mixture was stirred at 65° C. for 2 h. The reaction was cooled to room temperature, and EtOAc (60 ml) and aqueous solution of 3% LiCl (60 ml) were added to the mixture. The organic layer was washed with brine, dried (Na₂SO₄) and concentrated in vacuo. The residue was purified by silica gel chromatography to give the desired methyl ester product as a yellow solid (0.59 g). LC/MS=835).

### Step 8:

The methyl ester was dissolved in THF (20 ml), LiOH (0.6 g) in H₂O (10 ml) was added followed by addition of MeOH (1 ml). The mixture was kept stirring at room temperature for 20 h. Upon completion of the reaction, 40% TFA in H₂O was added to adjust pH to 7 at 0° C. The mixture was extracted with EtOAc. The combined organic layer was concentrated in vacuo then purified by prep HPLC to give the compound 7 as a yellow solid (714 mg, 79%). LC/MS=823 (M⁺+1). ¹H NMR (300 MHz, CD₃OD): δ 8.74 (s, 1H), 8.26 (d, 1H), 7.59 (d, 1H), 7.35 (s, 1H), 6.00-5.74 (m, 2H), 5.31-5.09 (dd, 2H). 4.69 (t, 1H), 4.52 (dd, 1H), 4.21-3.96 (m, 10H), 2.81 (m, 5H), 2.58 (m, 1H), 2.20 (m, 1H), 1.94 (m, 1H), 1.85-1.60 (m, 4H), 1.45 (m, 1H), 1.38 (s, 3H), 1.35 (s, 3H), 1.20 (m, 2H), 1.01 (s, 9H), 0.33 (m, 2H).

### Example 8

### Preparation of Compound 8

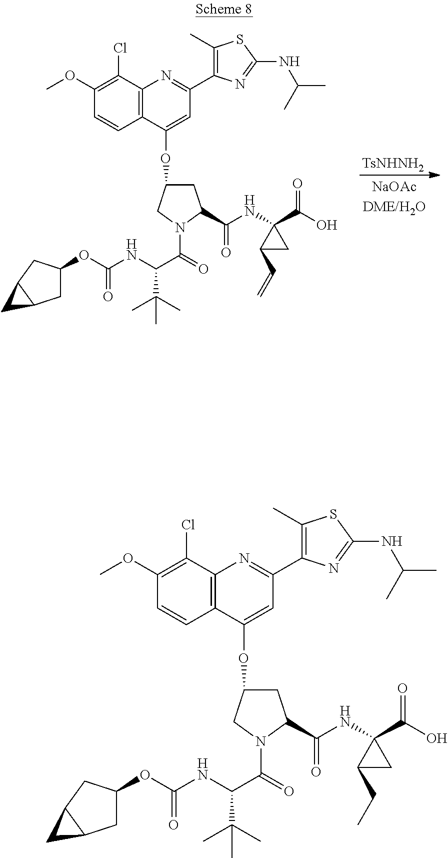

A mixture of compound 7 (320 mg, 0.388 mmol), p-TsNHNH₂ (542 mg, 2.91 mmol) and NaOAc (477 mg, 5.82 mmol) in a mixture of DME (10 mL) and H₂O (1 mL) was heated at 95° C. for 2 h. Upon completion of the reaction, it was cooled to room temperature, diluted with EtOAc (100 mL) and the pH was adjusted to 3 with 1N HCl. After separation of the organic and aqueous layers, the aqueous layer was back extracted with EtOAc. The organic layers were combined and concentrated. The crude product was purified by prep-HPLC to give compound 8 as a yellow solid (252 mg, 79%). LC/MS=825 (M⁺+1). ¹H NMR (300 MHz, CD₃OD): δ 8.24 (d, 1H), 7.58 (d, 1H), 7.31 (s, 1H), 5.72 (m, 1H), 4.71 (t, 1H), 4.58 (dd, 1H), 4.43 (t, 1H), 4.14 (s, 3H), 4.05 (m, 1H), 3.93 (m, 1H), 2.81 (s, 3H), 2.59 (m, 1H), 2.40 (dd, 2H), 1.94 (m, 1H), 1.80 (m, 1H), 1.64 (m, 3H), 1.52 (m, 1H), 1.38 (s, 3H), 1.36 (s, 3H), 1.27 (m, 2H), 1.01 (s, 9H), 0.33 (m, 2H).

### Example 9

### Preparation of Compound 9

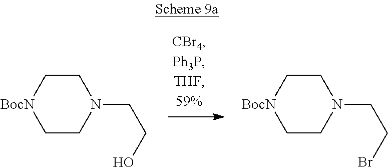

### Step 1:

Alcohol (3.42 g, 0.015 mmol) was dissolved in THF (55 mL). To this solution was added CBr₄ (5.47 g, 0.017 mmol). Ph₃P (4.46 g, 0.017 mmol) was dissolved in THF (20 mL) and slowly added to the reaction via an addition funnel. The reaction was stirred at room temperature for 16 h. The reaction was complete as determined by TLC. The reaction was diluted with hexanes and the white precipitate that formed was removed by filtration. More solids crashed in the filtrate. The mixture was transferred to a separatory funnel and the organic layer was extracted with sat. NaHCO₃ (aq.) (2×), dH₂O (2×) and brine (1×). The organic layer was dried over Na₂SO₄ and a small amount of MgSO₄. The drying agents were removed by vacuum filtration and bromide (2.59 g, 59% yield) was isolated from the filtrate by silica gel column chromatography, eluting with a mixture of EtOAc/hexanes. The bromide was isolated as a colorless oil that turns to a crystalline solid upon sitting. LC/MS=293.02 (M⁺+1).

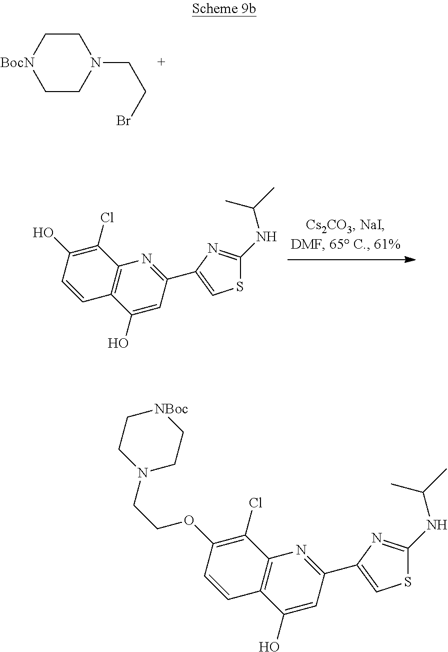

### Step 2:

An N₂ purged flask was charged with the bromide (738 mg, 2.5 mmol), bisphenol (1 g, 2.4 mmol), Cs₂CO₃ (1.21 g, 3.7 mmol) and NaI (72 mg, 0.48 mmol). To this mixture was added DMF (24 mL) and the heterogeneous mixture was heated in a preheated 65° C. oil bath. After 2 h very little of the bromide remained. Additional bromide (141 mg, 0.48 mmol) was added to the reaction and heating continued for 16 h. The reaction was complete, as determined by LC/MS, the next day. The reaction was cooled to room temperature and diluted with EtOAc. This mixture was extracted with 5% LiCl (aq.) basified with a small amount of sat. NaHCO₃(aq.) (2×) and brine (1×). The organic phase was then dried over Na₂SO₄ with a small amount of MgSO₄. After removal of the drying agents by vacuum filtration, the quinoline was isolated from the filtrate (800 mg, 61%) as a yellow-brown solid. LC/MS=548.26 (M⁺+1).

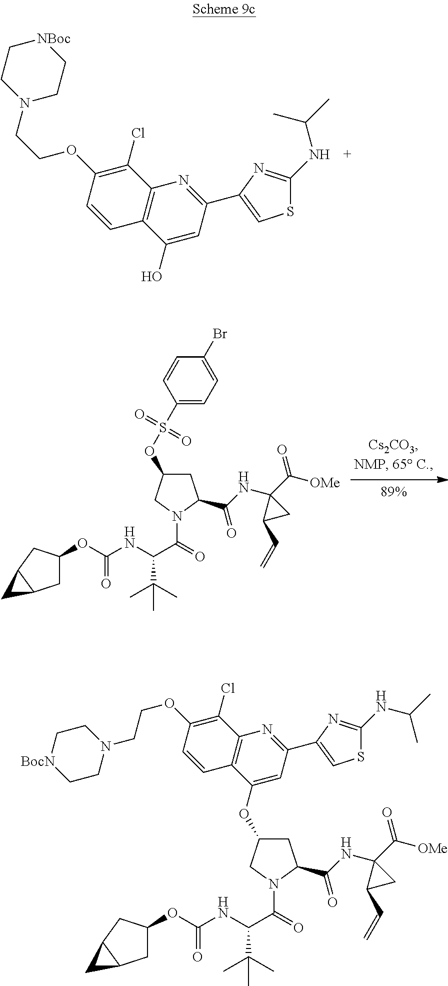

### Step 3:

An N₂ purged flask was charged with quinoline (800 mg, 1.46 mmol), brosylate (1.24 g, 1.75 mmol) and Cs₂CO₃ (570 mg, 1.75 mmol). To this mixture was then added NMP (14.6 mL) and the resulting heterogeneous mixture was heated in a preheated 65° C. oil bath. After 2 h the reaction shows a lot of progress. Heating continued for another 9 h and then the reaction was stirred at room temperature for 7 h. The reaction was diluted with EtOAc and the resulting mixture was extracted with 5% LiCl (aq.) (2×), and brine (1×). The organic phase was then dried over Na₂SO₄ and a small amount of MgSO₄. The drying agents were removed by vacuum filtration. The methyl ester was isolated from the filtrate by silica gel column chromatography as a slightly yellow-brown solid (1.33 g, 89%). LC/MS=1021.75 (M⁺+1).

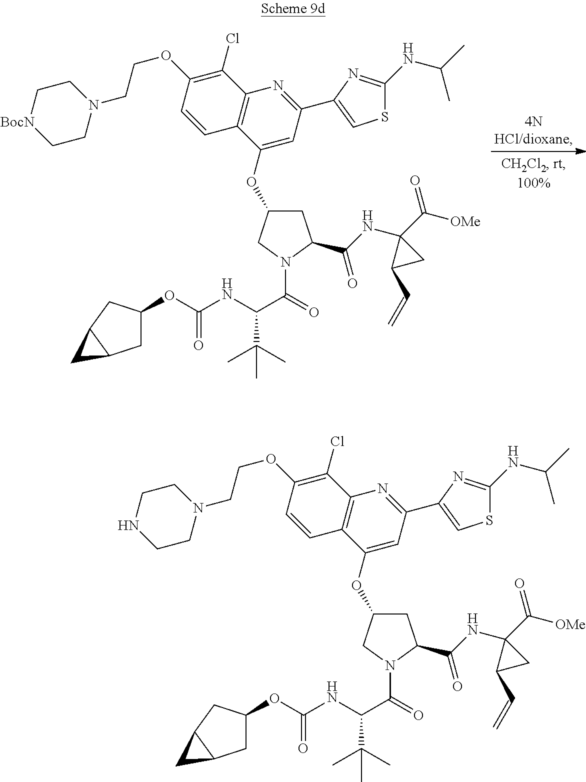

### Step 4:

The methyl ester (1.33 g, 1.3 mmol) was dissolved in CH₂Cl₂ (10 mL). This solution was cooled to 0° C. and 4N HCl in dioxanes (3.25 mL, 13 mmol) was added dropwise. The cold bath was then removed. The reaction was complete after 2 h, as determined by LC/MS. The reaction was concentrated, re-dissolved in CH₂Cl₂, and concentrated again. The residue was re-dissolved in CH₂Cl₂ again and then extracted with sat. NaHCO₃ (aq.) (1×). The organic phase was dried over Na₂SO₄ and a small amount of MgSO₄. The drying agents were removed by vacuum filtration and the filtrate was concentrated to yield the amine as slightly yellow foam (1.23 g, 100%). LC/MS=921.53 (M⁺+1).

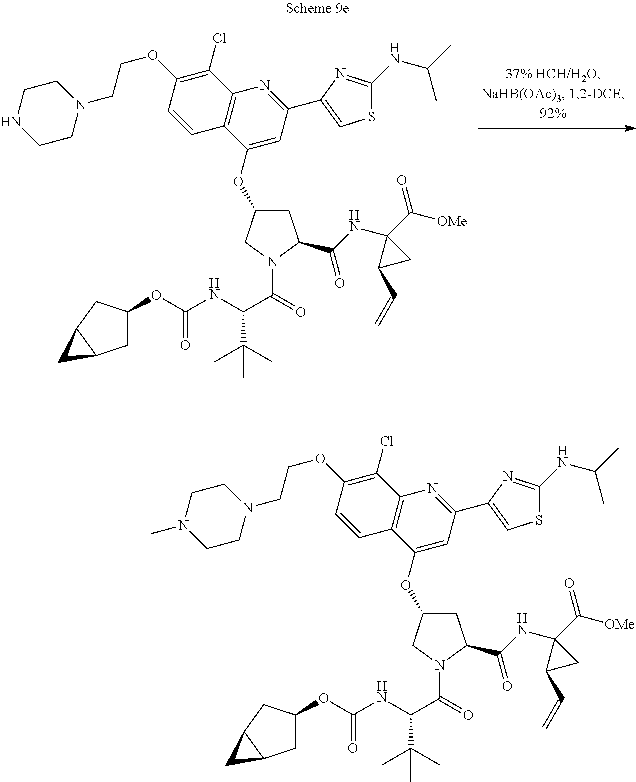

### Step 5:

The amine (608 mg, 0.66 mmol) was dissolved in 1,2-DCE (7 mL). To this solution was added 37% HCHO/H₂O (49 μL, 0.66 mmol). To this mixture was then added NaHB(OAc)₃ (560 mg, 2.64 mmol). The reaction was determined to be complete by LC/MS after 30 min. The reaction was quenched by the addition of sat. NaHCO₃ (aq.). The reaction was then diluted with EtOAc and extracted with sat. NaHCO₃ (aq.) (3×) and brine (1×). The organic phase was then dried over Na₂SO₄ and a small amount of MgSO₄. The drying agents were removed by vacuum filtration and the filtrate was concentrated. The residue was re-dissolved in MeOH and this solution was concentrated. This MeOH dissolution and concentration was repeated 2 more times to yield the methyl amine (569 mg, 92% yield) as a pink-orange foam. LC/MS=935.59 (M⁺+1).

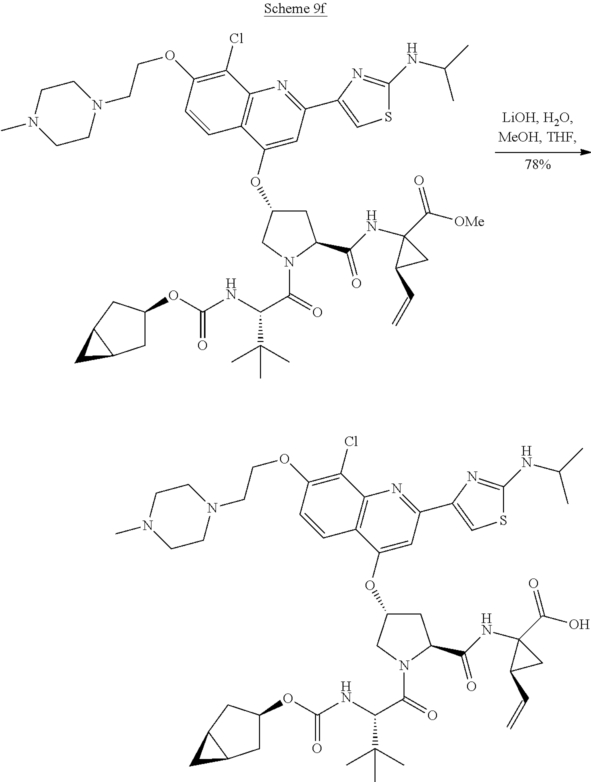

### Step 6:

The methyl ester (615 mg, 0.658 mmol) was dissolved in MeOH (2.2 mL) and THF (3.3 mL). This solution was cooled to 0° C. and a solution of LiOH.H₂O (138 mg, 3.29 mmol) in dH₂O (0.5 mL) was slowly added. The cold bath was then removed. After 3.5 h reaction was complete, as determined by LC/MS and HPLC. The reaction was cooled to 0° C. and quenched by the addition of 1N HCl. Compound 9 (590 mg, 78% yield) was isolated from the quenched reaction, by reverse phase HPLC, as a yellow solid. LC/MS=921.48 (M⁺+1). ¹H NMR (300 MHz, CD₃OD) δ 8.30 (d, J=10.2 Hz, 1H), 8.29 (s, 1H), 7.81 (s, 1H), 7.62 (d, J=10.2 Hz, 1H), 5.86 (dt, J=9.9, 16.8 Hz, 1H), 5.76 (s, 1H), 5.28 (d, J=17.1 Hz, 1H), 5.11 (d, J=10.2 Hz, 1H), 4.72 (t, J=8.4 Hz, 1H), 5.59 (d, J=5.4 Hz, 3H), 4.47 (t, J=6.3 Hz, 1H), 4.15 (s, 1H), 4.12-3.99 (m, 2H), 3.43 (s, 4H), 3.32-3.18 (m, 8H), 2.93 (s, 3H), 2.80 (dd, J=6.6, 14.1 Hz, 1H), 2.61 (m, 1H), 2.22 (dd, J=8.4, 9 Hz, 1H), 1.95 (m, 1H), 1.86-1.60 (m, 3H), 1.46 (dd, J=5.4, 9.3 Hz, 1H), 1.38 (d, J=6.6 Hz, 6H), 1.20 (m, 2H), 1.03 (s, 12H), 0.34 (m, 2H).

### Example 10

### Preparation of Compound 10

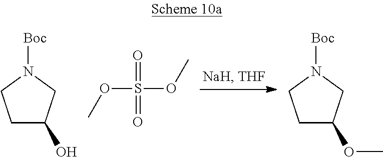

### Step 1:

An Ar purged flask was charged with 60% NaH (4.26 g, 106 mmol) and THF (60 mL). The alcohol (5 g, 26.67 mmol) in solution with THF (40 mL), was slowly added. The mixture was stirred at room temperature for 30 min then dimethylsulfate (5.07 mL, 53.3 mmol) was added. The reaction was stirred at room temperature overnight. The reaction was quenched with sat. NH₄Cl_{(aq)} (note: extreme outgassing). The mixture was stirred for 15 min and then the organic layer was separated from the aqueous layer. The aqueous layer was extracted with EtOAc. The organics were combined and concentrated under reduced pressure. The residue was taken up in EtOAc and washed with ½ sat NaHCO₃ (aq) followed by brine. The organics were dried over Na₂SO₄, filtered and solvent was removed under reduced pressure to afford the crude methyl ether (8.56 g, 42.03 mmol) as a colorless oil. LC/MS=202 (M⁺+1).

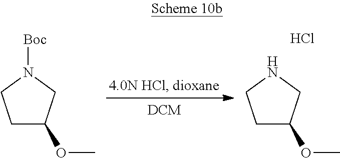

### Step 2:

An Ar purged flask was charged with the methyl ether (8.56 g, 42.03 mmol), followed by DCM (30 mL). 4.0 N HCl in dioxane (30 mL, 120 mmol) was slowly added. The reaction was stirred at room temperature for 2 h. The reaction was determined to be complete by LC/MS. The solvent was removed under reduced pressure to afford crude amine (7 g, 50 mmol) and used as is for the next step. LC/MS=102 (M⁺+1).

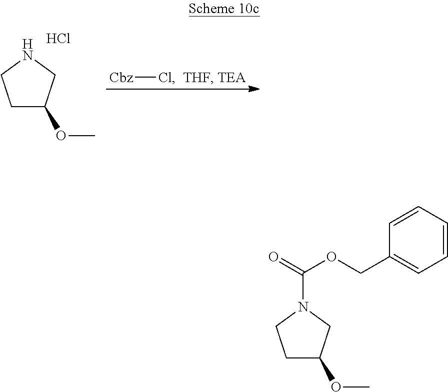

### Step 3:

An Ar purged flask was charged with amine (7 g, 50 mmol), THF (150 mL), CBz-Cl (10.7 mL, 76 mmol) and cool to 0° C. with an ice bath. Et₃N (21.1 mL, 150 mmol) was slowly added. The reaction was monitored by LCMS. The reaction is complete after 1 h. The solvent was removed under reduced pressure. The residue was taken up in EtOAc and washed with 0.5N HCl (aq), brine, and dried over Na₂SO₄. The solvent was removed under reduced pressure. The residue was dissolved in minimal DCM and purified by silica gel chromatography to afford the carbamate (4.52 g, 72% overall yield) as a white solid. LC/MS=236 (M⁺+1).

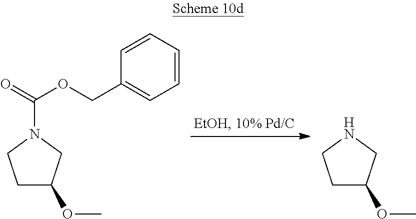

### Step 4:

An Ar purged flask was charged with carbamate (4.5 g, 19.1 mmol) and EtOH (50 mL). The flask was evacuated and re-pressurized with Ar. This process was repeated three times. The reaction flask was then charged with 10% Pd/C, and the flask was evacuated. The flask was then refilled with an atmosphere of H₂. The reaction was stirred at room temperature under an H₂ atmosphere, monitoring the reaction progress by LC/MS. The reaction was complete after 3 h. The solids were removed by vacuum filtration using a PTFE filter. The filtrate was concentrated under reduced pressure. The residue was coevaporated with EtOAc 3×50 mL to afford the crude amine (2.03 g, 20.0 mmol) as a colorless oil. LC/MS=102 (M⁺+1).

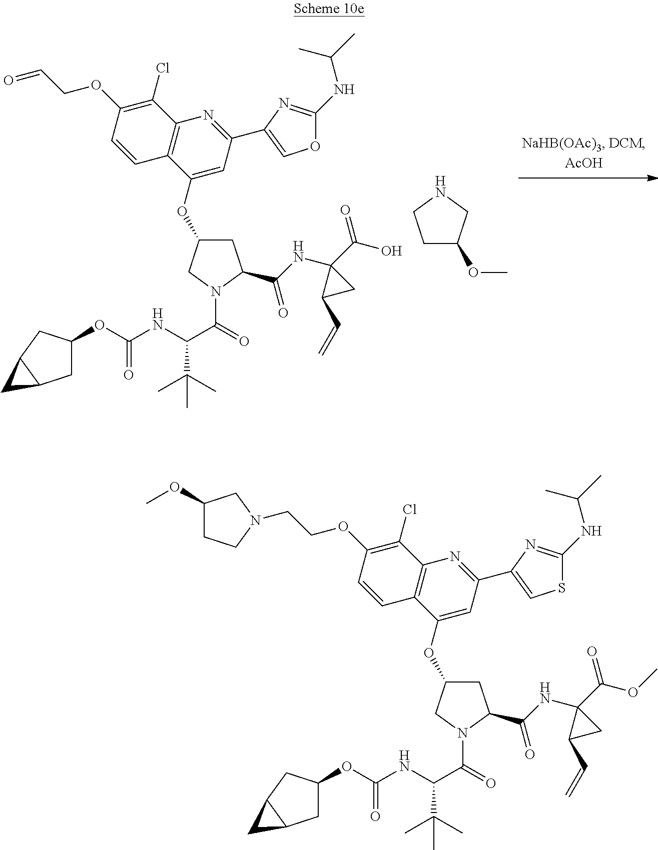

### Step 5:

The aldehyde (1.00 g, 1.17 mmol) was dissolved in DCM (15 mL), and the amine (176 mgs, 1.75 mmol) was added. To this mixture was then added NaHB(OAc)₃ (322 mg, 1.52 mmol), followed immediately by AcOH (20 μL, 0.3 mmol). The reaction was determined to be complete by LC/MS after 10 min. The reaction was quenched by the addition of ½ sat. NaHCO₃ (aq.). The reaction was then diluted with DCM and extracted with sat. NaHCO₃ (aq.) (3×) and brine (1×). The organic phase was then dried over Na₂SO₄. The drying agents were removed by vacuum filtration and the filtrate was concentrated. The residue was re-dissolved in MeOH and this solution was concentrated. This MeOH dissolution and concentration was repeated 2 more times to afford the crude methyl ester (968 mg, 88% yield) as a yellow solid. LC/MS=936 (M⁺+1).

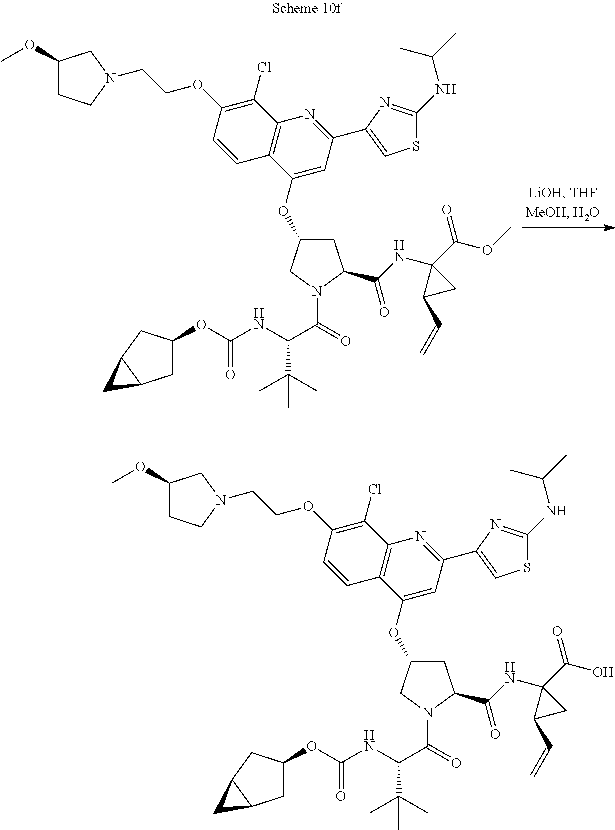

### Step 6:

The ester (968 mg, 1.03 mmol) was dissolved in a mixture of THF (10 mL) and MeOH (6 mL). LiOH (200 mg, 4.67 mmol) was dissolved in dH₂O (3 mL) and this was slowly added to the solution of ester in THF/MeOH, which had been cooled to 0° C. Upon complete addition the ice bath was removed. After 3 h the reaction was complete. The reaction was cooled to 0° C. and neutralized with 2N HCl. Compound 10 was extracted into EtOAc, which was then extracted with 1N HCl, and brine. The organics were then dried over Na₂SO₄. The solids were removed by filtration and the volatile organics removed under reduced pressure. Compound 10 (900 mgs) was isolated as a yellow solid. LC/MS=922 (M⁺+1).

### Example 11

### Preparation of Compound 11

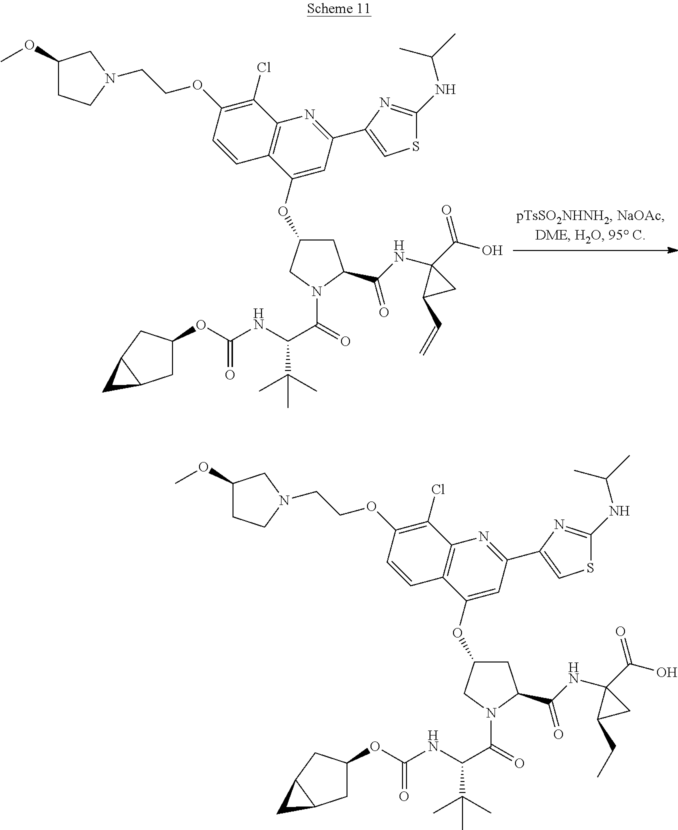

Compound 10 (900 mg, 0.977 mmol) was dissolved in DME (5 mL). To this solution was added dH₂O (1 mL), pTolSO₂NHNH₂ (920 mg, 4.93 mmol) and NaOAc (850 mg, 10.36 mmol). The reaction flask was then placed in a preheated 95° C. oil bath for 2 h. The reaction was determined to be complete by LC/MS. The reaction was cooled to room temperature and a small amount of MeOH was added to make the reaction mono-phasic. The reaction was then filtered and 11 (686 mg, 76% yield) was isolated from the filtrate, by reverse phase HPLC, as a yellow solid. LC/MS=924 (M⁺+1).

### Example 12

### Preparation of Compound 12

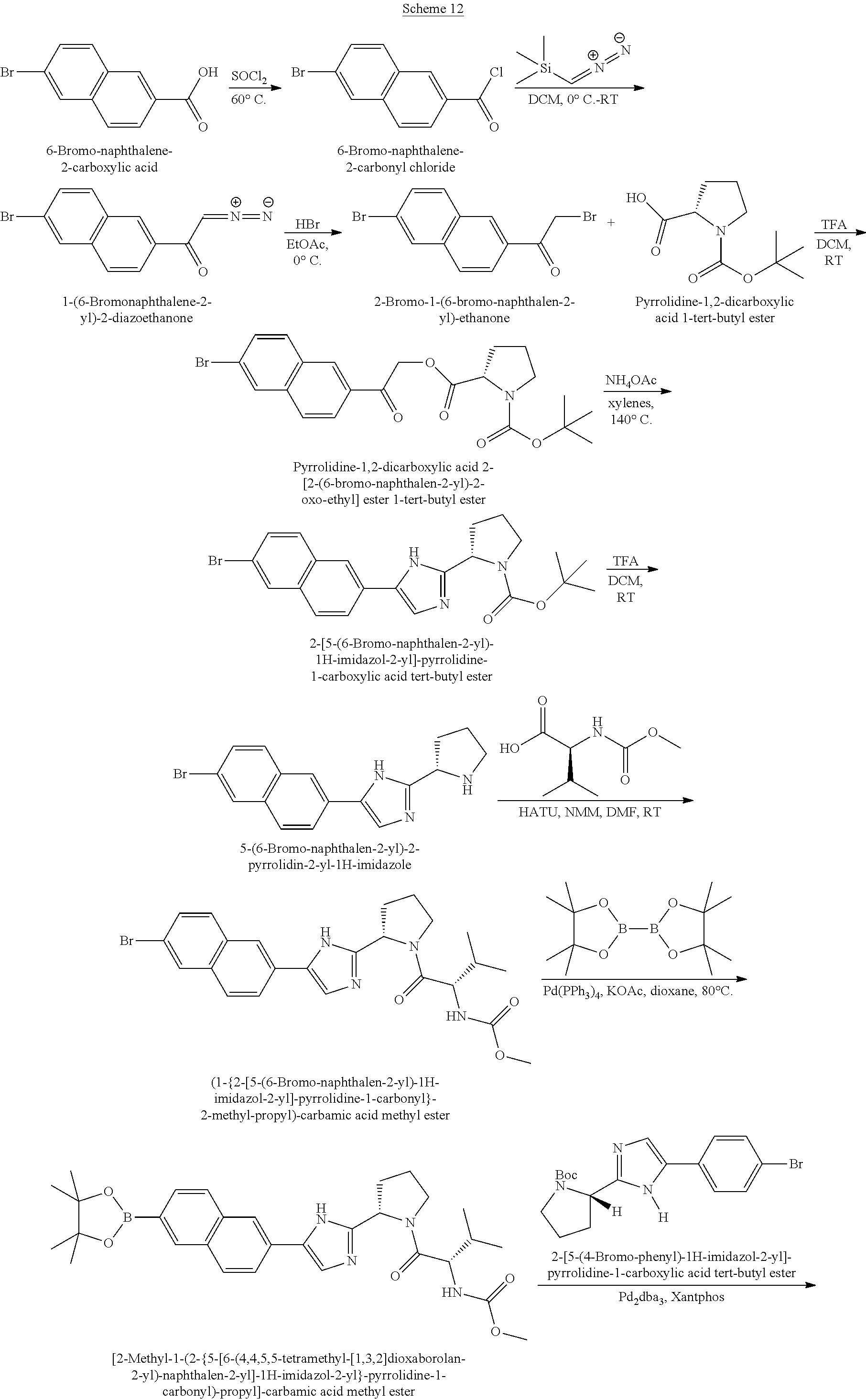

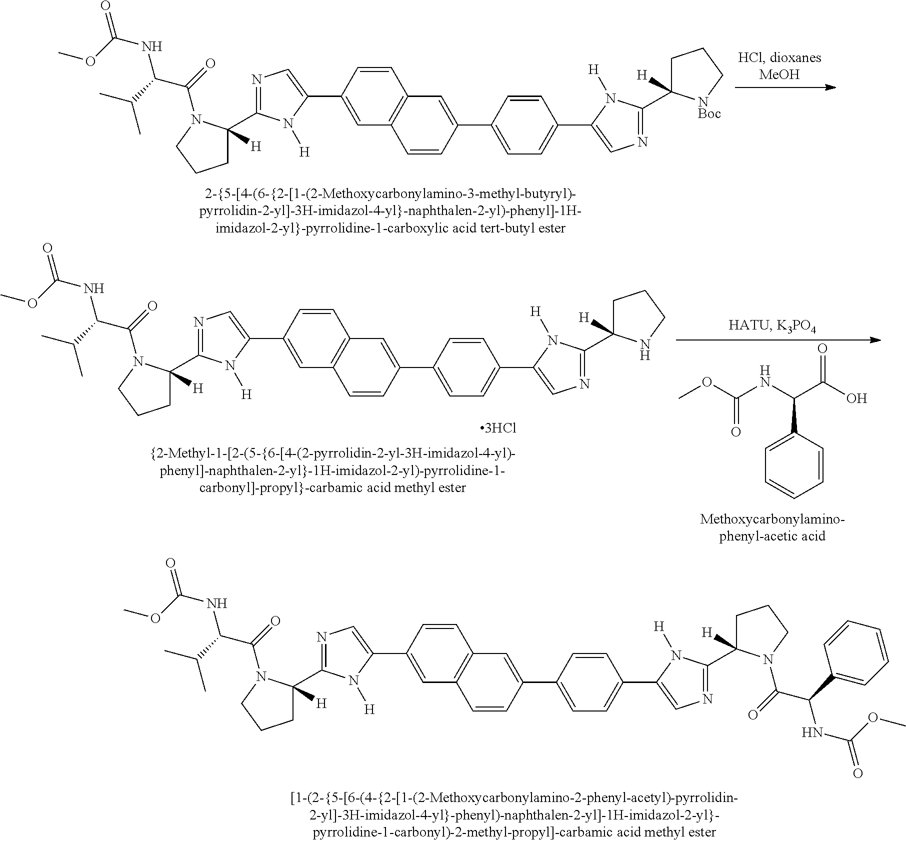

6-Bromo-naphthalene-2-carbonyl chloride: 6-Bromonaphthalene-2-carboxylic acid (25.1 g) was suspended in thionyl chloride (200 mL), stirred at 60° C. for 16 hours and evaporated under vacuum. Solid was dissolved in dichloromethane (50 mL) and evaporated under vacuum, giving 6-bromonaphthalene-2-carbonyl chloride (27.0 g, crude) as a white solid.

1-(6-Bromo-naphthalen-2-yl)-2-diazo-ethanone: 6-Bromonaphthalene-2-carbonyl chloride (27.0 g, crude) was dissolved in dichloromethane (330 mL) and cooled to 0° C. TMS diazomethane solution (100 mL, 2 M in DCM) was added, and ice bath was removed. Reaction mixture was stirred for 16 hours and evaporated under vacuum, giving 1-(6-bromonaphthalen-2-yl)-2-diazoethanone (34.7 g, crude) as an orange solid.

2-Bromo-1-(6-bromo-naphthalen-2-yl)-ethanone: 1-(6-Bromonaphthalen-2-yl)-2-diazoethanone (34.7 g) were dissolved in ethyl acetate (500 mL), and hydrobromic acid solution (21.1 mL, 5.7 M in acetic acid) was added at 0° C. Reaction mixture was stirred 3 hours, NaHCO₃ solution (200 mL) was added, and mixture was stirred 10 minutes. Ethyl acetate solution was extracted twice with NaHCO₃ solution (50 mL), once with brine (50 mL), and evaporated under vacuum, giving 2-bromo-1-(6-bromonaphthalen-2-yl)-ethanone (33.0 g, crude) as a tan solid.

Pyrrolidine-1,2-dicarboxylic acid 2-[2-(6-bromo-naphthalen-2-yl)-2-oxo-ethyl]ester 1-tert-butyl ester: Pyrrolidine-1,2-dicarboxylic acid 1-tert-butyl ester (24.0 g) was dissolved in acetonitrile (330 mL), and triethylamine (15.6 mL) was added. A solution of 2-bromo-1-(6-bromonaphthalen-2-yl)-ethanone (33.0 g) in acetonitrile (170 mL) were added. Reaction mixture was stirred over 3 days and evaporated under vacuum. Oil was dissolved in dichloromethane (100 mL), extracted with water (50 mL) and with NaHCO₃ solution (50 mL), and evaporated under vacuum to a concentrated liquid. Solution was purified by chromatography (0-30% ethyl acetate:hexane) and evaporated under vacuum, giving pyrrolidine-1,2-dicarboxylic acid 2-[2-(6-bromo-naphthalen-2-yl)-2-oxo-ethyl]ester 1-tert-butyl ester (39.2 g, 84%) as a tan solid.

2-[5-(6-Bromo-naphthalen-2-yl)-1H-imidazol-2-yl]-pyrrolidine-1-carboxylic acid tert-butyl ester: Pyrrolidine-1,2-dicarboxylic acid2-[2-(6-bromonaphthalen-2-yl)-2-oxo-ethyl]ester 1-tert-butyl ester (39.0 g) and ammonium acetate (40.1 g) were suspended in xylenes (420 mL). The reaction mixture was stirred at 140° C. for 15 hours and evaporated under vacuum. Solid was dissolved in dichloromethane (300 mL), extracted twice with water (50 mL) and once with brine (50 mL), and evaporated under vacuum, giving 2-[5-(6-bromonaphthalen-2-yl)-1H-imidazol-2-yl]-pyrrolidine-1-carboxylic acid tert-butyl ester (30.3 g, 81%) as an off-white solid.

5-(6-Bromo-naphthalen-2-yl)-2-pyrrolidin-2-yl-1H-imidazole: 2-[5-(6-Bromonaphthalen-2-yl)-1H-imidazol-2-yl]-pyrrolidine-1-carboxylic acid tert-butyl ester (5.03 g) was dissolved in dichloromethane (75 mL), and trifluoroacetic acid (25 mL) was added. The reaction mixture was stirred at ambient temperature for 5 hours and evaporated under vacuum. Solid was dissolved in dichloromethane (50 mL) and extracted with saturated NaHCO₃ solution (50 mL). Solid was collected by vacuum filtration, washed with dichloromethane, and dried under vacuum, giving 2-[5-(6-bromonaphthalen-2-yl)-1H-imidazol-2-yl]-pyrrolidine-1-carboxylic acid tert-butyl ester (3.80 g, 98%) as an off-white solid.

(1-{2-[5-(6-Bromonaphthalen-2-yl)-1H-imidazol-2-yl]-pyrrolidine-1-carbonyl}-2-methyl-propyl)-carbamic acid methyl ester: 2-[5-(6-Bromonaphthalen-2-yl)-1H-imidazol-2-yl]-pyrrolidine-1-carboxylic acid tert-butyl ester (3.80 g), 2-methoxycarbonylamino-3-methyl-butyric acid (2.21 g), and HATU (5.06 g) were dissolved in anhydrous DMF (75 mL), and N-methylmorpholine (2.68 mL) was added. The reaction mixture was stirred at ambient temperature for 16 hours and evaporated under vacuum. The oil was dissolved in dichloromethane, purified by chromatography (0-100% ethyl acetate:hexanes), and evaporated under vacuum, giving (1-{2-[5-(6-bromo-naphthalen-2-yl)-1H-imidazol-2-yl]-pyrrolidine-1-carbonyl}-2-methyl-propyl)-carbamic acid methyl ester (0.814 g, 72%) as an off-white solid.

[2-Methyl-1-(2-{5-[6-(4,4,5,5-tetramethyl-[1,3,2]dioxaborolan-2-yl]-naphthalen-2-yl]-1H-imidazol-2-yl}-pyrrolidine-1-carbonyl)-propyl]-carbamic acid methyl ester: (1-{2-[[5-(6-Bromonaphthalen-2-yl]-1H-imidazol-2-yl]-pyrrolidine-1-carbonyl}-2-methyl-propyl)-carbamic acid methyl ester (3.02 g), bis-(pinocolato)diborane (3.18 g), and potassium acetate (1.52 g) were dissolved in 1,4-dioxane (40 mL), and solution was degassed with nitrogen. Pd(PPh₃)₄ (0.285 g) was added, and the reaction mixture was stirred at 80° C. for 20 hours and evaporated under vacuum. Solid was dissolved in dichloromethane (50 mL), extracted with saturated NaHCO₃ solution (50 mL), and evaporated under vacuum. The oil was dissolved in dichloromethane, purified by chromatography (0-10% isopropanol:dichloromethane), and evaporated under vacuum, giving (1-{2-[5-(6-bromonaphthalen-2-yl)-1H-imidazol-2-yl]-pyrrolidine-1-carbonyl}-2-methyl-propyl)-carbamic acid methyl ester (3.65 g, crude) as a yellow solid.

2-{5-[4-(6-{2-[1-(2-Methoxycarbonylamino-3-methyl-butyryl)-pyrrolidin-2-yl]-3H-imidazol-4-yl}-naphthalen-2-yl)-phenyl]-1H-imidazol-2-yl}-pyrrolidine-1-carboxylic acid tert-butyl ester. To a solution of 2-[5-(4-Bromo-phenyl)-1H-imidazol-2-yl]-pyrrolidine-1-carboxylic acid tert-butyl ester (1.00 g, 2.5 mmol) and [2-Methyl-1-(2-{5-[6-(4,4,5,5-tetramethyl-[1,3,2]dioxaborolan-2-yl)-naphthalen-2-yl]-1H-imidazol-2-yl}-pyrrolidine-1-carbonyl)-propyl]-carbamic acid methyl ester (1.97 g, 3.6 mmol, 1.5 equiv.) in DME (12.5 mL) was added K₃PO₄ (aqueous, 2 M, 3.9 mL, 7.8 mmol, 3 equiv.), Pd₂dba₃ (0.12 g, 0.13 mmol, 0.05 equiv.), and Xantphos (0.15 g, 0.26 mmol, 0.1 equiv.). The slurry was degassed with argon for 5 minutes and heated to 80° C. for 18 hours. The resulting reaction mixture was diluted with EtOAc/MeOH (10:1) and filtered through Celite. The solution was washed with water and brine. The aqueous layer was back-extracted with EtOAc and the combined organic layers were dried over Na₂SO₄ and concentrated. The crude oil was purified by column chromatography (SiO₂, 50→100% EtOAc in Hexanes) to provide 2-{5-[4-(6-{2-[1-(2-Methoxycarbonylamino-3-methyl-butyryl)-pyrrolidin-2-yl]-3H-imidazol-4-yl}-naphthalen-2-yl)-phenyl]-1H-imidazol-2-yl}-pyrrolidine-1-carboxylic acid tert-butyl ester (0.93 g, 49%) as a yellow powder. LCMS-ESI⁺: calc'd for C₄₂H₄₉N₇O₅: 731.4 (M⁺); Found: 732.9 (M+H⁺).

{2-Methyl-1-[2-(5-{6-[4-(2-pyrrolidin-2-yl-3H-imidazol-4-yl)-phenyl]-naphthalen-2-yl}-1H-imidazol-2-yl)-pyrrolidine-1-carbonyl]-propyl}-carbamic acid methyl ester. To a slurry of 2-{5-[4-(6-{2-[1-(2-Methoxycarbonylamino-3-methyl-butyryl)-pyrrolidin-2-yl]-3H-imidazol-4-yl}-naphthalen-2-yl)-phenyl]-1H-imidazol-2-yl}-pyrrolidine-1-carboxylic acid tert-butyl ester (0.1 g, 0.14 mmol) in MeOH (0.15 mL) was added HCl in dioxanes (4 M, 0.7 mL). The resulting solution was stirred at room temperature for 1 hour and diluted with Et₂O. The resulting precipitate was filtered and dried to provide {2-Methyl-1-[2-(5-{6-[4-(2-pyrrolidin-2-yl-3H-imidazol-4-yl)-phenyl]-naphthalen-2-yl}-1H-imidazol-2-yl)-pyrrolidine-1-carbonyl]-propyl}-carbamic acid methyl ester trihydrochloric acid salt (0.09 g, 87%) as a white powder. LCMS-ESI⁺: calc'd for C₃₇H₄₁N₇O₃: 631.3 (M⁺); Found: 632.7 (M+H⁺).

[1-(2-{5-[6-(4-{2-[1-(2R)-(2-Methoxycarbonylamino-2-phenyl-acetyl)-pyrrolidin-2-yl]-3H-imidazol-4-yl}-phenyl)-naphthalen-2-yl]-1H-imidazol-2-yl}-pyrrolidine-1-carbonyl)-2-methyl-propyl]-carbamic acid methyl ester. To a slurry of {2-Methyl-1-[2-(5-{6-[4-(2-pyrrolidin-2-yl-3H-imidazol-4-yl)-phenyl]-naphthalen-2-yl}-1H-imidazol-2-yl)-pyrrolidine-1-carbonyl]-propyl}-carbamic acid methyl ester (0.045 g, 0.06 mmol) and (R)-methoxycarbonylamino-phenyl-acetic acid (0.02 g, 0.09 mmol, 1.5 equiv.) in CH₂Cl₂ (0.6 mL) was added HATU (0.03 g, 0.08, 1.25 equiv.) and K₃PO₄ (0.05 g, 0.22 mmol, 3 equiv.). The reaction mixture was stirred at room temperature for 18 hours and diluted with CH₂Cl₂. The salts were filtered and the filtrate was concentrated. The crude oil was purified by preparative HPLC (Gemini, 15→40% MeCN in H₂O (0.1% formic acid)) and lyophilized to provide [1-(2-{5-[6-(4-{2-[1-(2S)-(2-Methoxycarbonylamino-2-phenyl-acetyl)-pyrrolidin-2-yl]-3H-imidazol-4-yl}-phenyl)-naphthalen-2-yl]-1H-imidazol-2-yl}-pyrrolidine-1-carbonyl)-2-methyl-propyl]-carbamic acid methyl ester (0.03 g, 65%) as a white powder. LCMS-ESI⁺: calc'd for C₄₇H₅₀N₈O₆: 822.4 (M⁺); Found: 823.8 (M+H⁺). ¹H-NMR: 400 MHz, (CDCl₃) δ: (Mixture of rotomers) 7.62-8.02 (m, 9H), 7.36-7.43 (m, 6H), 7.22 (s, 2H), 6.01 (s, 1H), 5.29-5.53 (m, 4H), 4.35 (t, 1H), 3.73-3.87 (m, 2H), 3.68 (s, 3H), 3.63 (s, 3H), 3.22 (q, 2H), 2.82-2.96 (m, 2H), 2.37 (m, 1H), 2.23 (m, 2H), 1.90-2.11 (m, 4H), 0.87-0.93 (m, 6H).

### Example 13

### Preparation of Compound 13

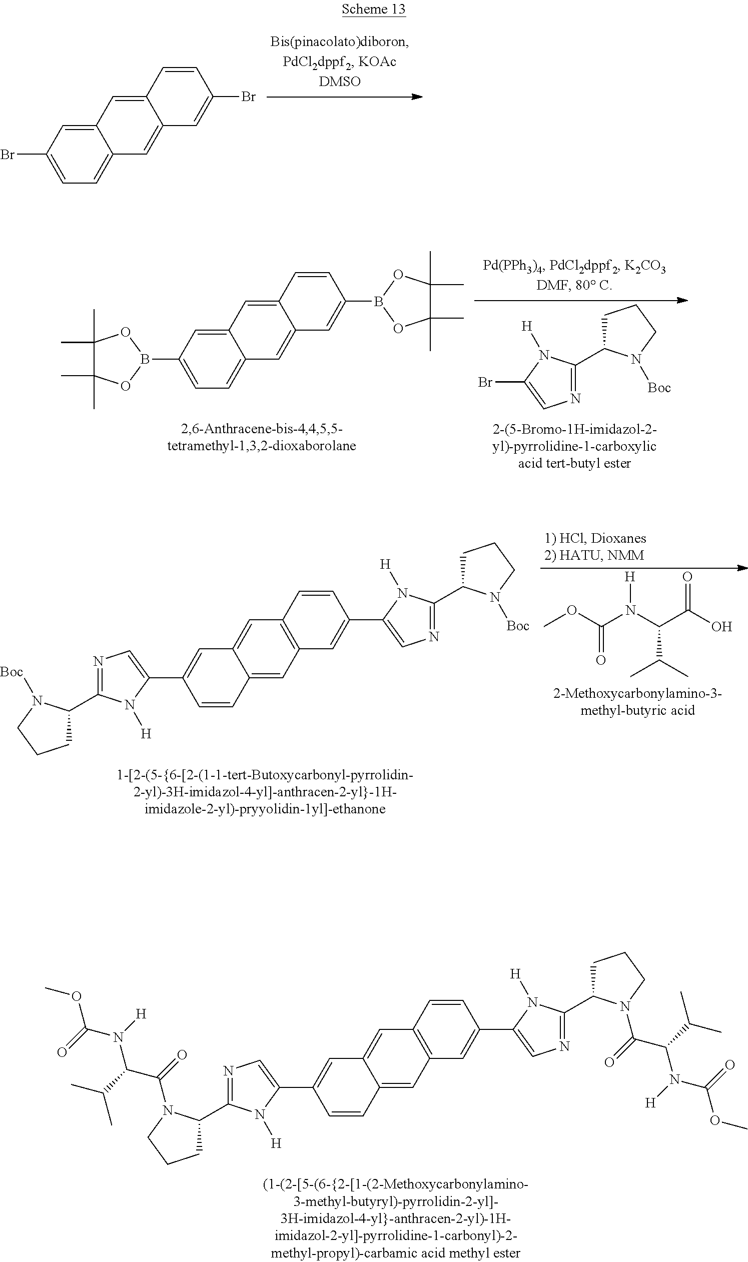

2,6-Anthracene-bis-4,4,5,5-tetramethyl-1,3,2-dioxaborolane: A mixture of 2,6-dibromoanthracene (500 mg, 1.49 mmol), bis(pinacolato)diboron (756 mg, 2.98 mmol) and KOAc (585 mg, 5.96 mmol) in DMSO (10 mL) was degassed with N₂ gas for 20 minutes. To the degassed solution was added PdCl₂dppf₂ (55 mg, 0.075 mmol) then the reaction was heated to 80° C. overnight. After cooling to room temperature, the reaction was poured into H₂O and extracted with CH₂Cl₂. The organic phase was collected then washed with H₂O and brine. After drying over Na₂SO₄, the organic phase was concentrated then purified by silica gel chromatography (30-100% CH₂Cl₂-hexanes gradient) to afford 2,6-anthracene-bis-4,4,5,5-tetramethyl-1,3,2-dioxaborolane (241 mg, 0.56 mmol, 38% yield). ¹H-NMR: 400 MHz, (DMSO-d₆) δ: 8.57 (s, 2H), 8.46 (s, 2H), 8.00 (d, 2H), 7.79 (d, 2H).

1-[2-(5-{6-[2-(1-1-tert-Butoxycarbonyl-pyrrolidin-2-yl)-3H-imidazol-4-yl]-anthracen-2-yl}-1H-imidazol-2-yl)-pyrrolidin-1-yl]-ethanone: A solution of 2,6-anthracene-bis-4,4,5,5-tetramethyl-1,3,2-dioxaborolane (241 mg, 0.56 mmol), 2-(5-bromo-1H-imidazol-2-yl)-pyrrolidine-1-carboxylic acid tert-butyl ester (531 mg, 1.68 mmol) and aq K₂CO₃ (1.12 mL of a 2M solution, 2.24 mmol) in toluene (6 mL) and DMF (1 mL) was degassed with N₂ gas for 20 minutes. To the degassed solution was added Pd(PPh₃)₄ (32 mg, 0.028 mmol) and PdCl₂dppf (21 mg, 0.028 mmol) then the reaction was heated to 80° C. overnight. After cooling to room temperature, the reaction was concentrated. The crude material was diluted with EtOAc then washed with saturated NaHCO₃. The aqueous phase was back-extracted two times then the organic layers were combined, dried over Na₂SO₄, and concentrated. The crude product was purified by reverse phase preparative HPLC (20-80% MeCN—H₂O; 0.1% formic acid modifier) to afford 1-[2-(5-{6-[2-(1-1-tert-butoxycarbonyl-pyrrolidin-2-yl)-3H-imidazol-4-yl]-anthracen-2-yl}-1H-imidazol-2-yl)-pyrrolidin-1-yl]-ethanone (117 mg, 0.18 mmol, 32% yield). LCMS-ESI⁺: calc'd for C₃₈H₄₅N₆O₄: 649.4 (M+H⁺); Found: 648.9 (M+H⁺).

(1-{2-[5-(6-{2-[1-(2-Methoxycarbonylamino-3-methyl-butyryl)-pyrrolidin-2-yl]-3H-imidazol-4-yl}-anthracen-2-yl)-1H-imidazol-2-yl]-pyrrolidine-1-carbonyl}-2-methyl-propyl)-carbamic acid methyl ester: To 1-[2-(5-{6-[2-(1-1-tert-butoxycarbonyl-pyrrolidin-2-yl)-3H-imidazol-4-yl]-anthracen-2-yl}-1H-imidazol-2-yl)-pyrrolidin-1-yl]-ethanone (117 mg, 0.18 mmol) in dioxanes (5 mL) was added 4N HCl in dioxanes (180 uL, 0.72 mmol). The suspension overnight then concentrated to afford the HCl salt of the crude amine. To the amine in DMF (3 mL) was added N-methylmorpholine (119 uL, 1.08 mmol). After all the material dissolved, 2-methoxycarbonylamino-3-methyl-butyric acid (76 mg, 0.43 mmol) and HATU (151 mg, 0.40 mmol) were added. After stirring overnight the reaction was quenched with AcOH then purified by reverse phase preparative HPLC (15-70% MeCN—H₂O; 0.1% formic acid modifier) to afford (1-{2-[5-(6-{2-[1-(2-methoxycarbonylamino-3-methyl-butyryl)-pyrrolidin-2-yl]-3H-imidazol-4-yl}-anthracen-2-yl)-1H-imidazol-2-yl]-pyrrolidine-1-carbonyl}-2-methyl-propyl)-carbamic acid methyl ester (46 mg, 0.098 mmol, 54% yield). ¹H-NMR: 400 MHz, (DMSO-d₆) δ: 11.84 (s, 2H), 8.38 (s, 2H), 8.31 (s, 2H), 8.00 (d, 2H), 7.86 (d, 2H), 7.62 (s, 2H), 7.30 (d, 2H), 5.12 (m, 2H), 4.10 (m, 2H), 3.84 (m, 4H), 3.55 (s, 6H), 2.18-1.95 (m, 10 H), 0.96 (d, 6H), 0.88 (d, 6H), LCMS-ESI⁺: calc'd for C₄₂H₅₁N₈O₆: 763.4 (M+H⁺); Found: 763.1 (M+H⁺).

### Example 14

### Preparation of Compound 14

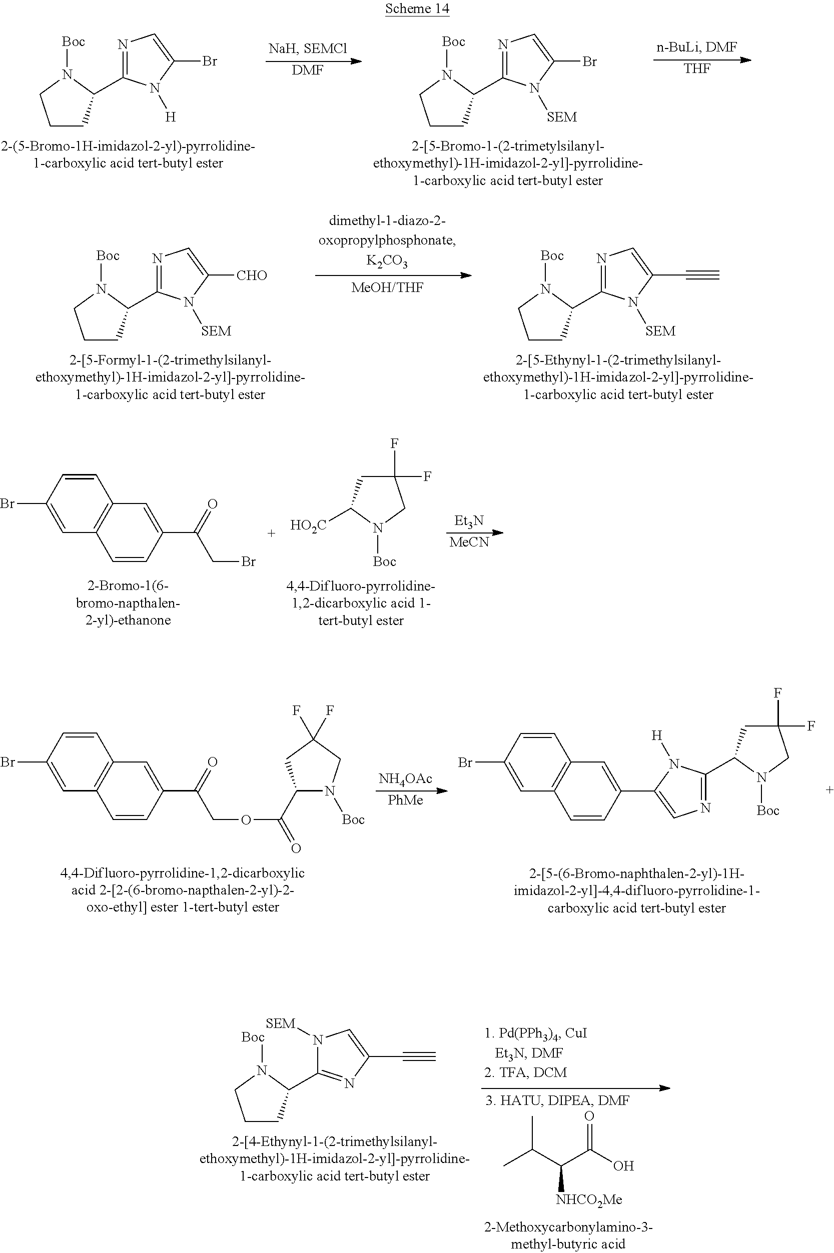

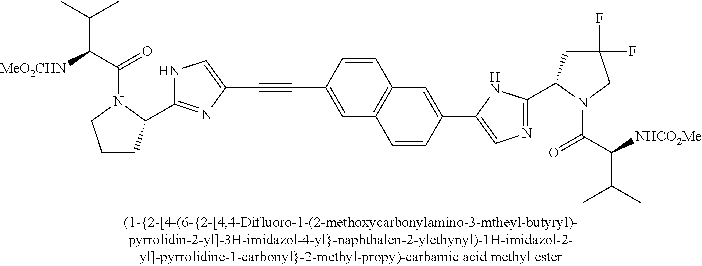

2-[5-Bromo-1-(2-trimethylsilanyl-ethoxymethyl)-1H-imidazol-2-yl]-pyrrolidine-1-carboxylic acid tert-butyl ester: 2-(5-Bromo-1H-imidazol-2-yl)-pyrrolidine-1-carboxylic acid tert-butyl ester (4 g, 12.65 mmol) was dissolved in DMF and cooled to 0° C. NaH (658 mg of 60% mineral oil dispersion, 16.45 mmol) was added and the reaction mixture was aged for 13 min before addition of SEMCI (2.7 mL, 15.18 mmol) and warming to room temperature. After 16 h, the reacton was quenched by water, diluted with ethyl acetate (300 mL) and washed with water and brine. The organic phase was dried over magnesium sulfate and concentrated. The crude residue was purified by silica column chromatography (10% to 30% EtOAc/hexanes) to afford 2-[5-Bromo-1-(2-trimethylsilanyl-ethoxymethyl)-1H-imidazol-2-yl]-pyrrolidine-1-carboxylic acid tert-butyl ester (4.67 g, 83%).

2-[5-Formyl-1-(2-trimethylsilanyl-ethoxymethyl)-1H-imidazol-2-yl]-pyrrolidine-1-carboxylic acid tert-butyl ester: 2-[5-Bromo-1-(2-trimethylsilanyl-ethoxymethyl)-1H-imidazol-2-yl]-pyrrolidine-1-carboxylic acid tert-butyl ester (3.804 g, 8.52 mmol) was dissolved in THF (42 mL) and cooled to −78° C. n-BuLi (3.4 mL of a 2.5 M hexane solution, 8.52 mmol) was added dropwise over 3 min. After 65 min, DMF (4 mL) was added and the reaction mixture was warmed to room temperature. After stirring at room temperature for 75 min, a saturated aqueous solution of ammonium chloride (50 mL) was added and the entire content of the flask was poured into saturated aqueous sodium bicarbonate. The aqueous phase was extracted 3 times with diethyl ether. The combined organic layers were dried over magnesium sulfate, concentrated and purified by silica column chromatography (30% to 70% EtOAc/hexanes) to provide 2-[5-Formyl-1-(2-trimethylsilanyl-ethoxymethyl)-1H-imidazol-2-yl]-pyrrolidine-1-carboxylic acid tert-butyl ester (1.50 g, 45%).

2-[5-Ethynyl-1-(2-trimethylsilanyl-ethoxymethyl)-1H-imidazol-2-yl]-pyrrolidine-1-carboxylic acid tert-butyl ester: 2-[5-Formyl-1-(2-trimethylsilanyl-ethoxymethyl)-1H-imidazol-2-yl]-pyrrolidine-1-carboxylic acid tert-butyl ester (1.625 g, 4.11 mmol) and dimethyl-1-diazo-2-oxopropylphosphonate (1.056 g, 5.50 mmol) were dissolved in 1:1 MeOH/THF (10 mL) and potassium carbonate (1.14 g, 8.25 mmol) was added. After stirring for 200 min, more potassium carbonate (1.14 g, 8.25 mmol) was added. 45 min later, the reaction mixture was poured into 100 mL 1:1 water/saturated aqueous sodium bicarbonate. The aqueous phase was extracted 3 times with diethyl ether. The combined organic phases were dried with magnesium sulfate and concentrated. The crude residue was purified by silica column chromatography (20% to 45% EtOAc/hexanes) to afford 2-[5-Ethynyl-1-(2-trimethylsilanyl-ethoxymethyl)-1H-imidazol-2-yl]-pyrrolidine-1-carboxylic acid tert-butyl ester (1.234 g, 77%).

4,4-Difluoro-pyrrolidine-1,2-dicarboxylic acid 2-[2-(6-bromo-naphthalen-2-yl)-2-oxo-ethyl]ester 1-tert-butyl ester: 2-Bromo-1-(6-bromo-naphthalen-2-yl)-ethanone (1 g, 3.07 mmol) and 4,4-Difluoro-pyrrolidine-1,2-dicarboxylic acid 1-tert-butyl ester (849 mg, 3.38 mmol) were suspended in MeCN (15 mL) and treated with Et₃N (0.45 mL, 3.22 mmol). After stirring o/n, the reaction mixture was concentrated. The resulting residue was dissolved in EtOAc and washed with water, saturated aqueous NaHCO₃ and brine. The organic layer was dried over MgSO₄, filtered and concentrated. The crude material was purified by silica column chromatography (0% to 20% EtOAc/Hex) to provide the title compound (1.27 g, 83%).

2-[5-(6-Bromo-naphthalen-2-yl)-1H-imidazol-2-yl]-4,4-difluoro-pyrrolidine-1-carboxylic acid tert-butyl ester: 4,4-Difluoro-pyrrolidine-1,2-dicarboxylic acid 2-[2-(6-bromo-naphthalen-2-yl)-2-oxo-ethyl]ester 1-tert-butyl ester (1.2 g, 2.41 mmol) was treated with NH₄OAc (3.72 g, 96.4 mmol) and PhMe (48 mL). The reaction mixture was refluxed with stirring for 18 h. After this period, it was cooled to room temperature, diluted with EtOAc and washed with saturated aqueous NaHCO₃ and brine. Filtration and concentration provided a crude residue that was purified by silica column chromatography (20% to 60% EtOAc/Hex) to provide the title compound (803 mg, 70%). (1-{2-[4-(6-{2-[4,4-Difluoro-1-(2-methoxycarbonylamino-3-methyl-butyryl)-pyrrolidin-2-yl]-3H-imidazol-4-yl}-naphthalen-2-ylethynyl)-1H-imidazol-2-yl]-pyrrolidine-1-carbonyl}-2-methyl-propyl)-carbamic acid methyl ester: 2-[4-Ethynyl-1-(2-trimethylsilanyl-ethoxymethyl)-1H-imidazol-2-yl]-pyrrolidine-1-carboxylic acid tert-butyl ester (199 mg, 0.508 mmol), 2-[5-(6-Bromo-naphthalen-2-yl)-1H-imidazol-2-yl]-4,4-difluoro-pyrrolidine-1-carboxylic acid tert-butyl ester (364 mg, 0.762 mmol), Pd(PPh₃)₄ (118 mg, 0.102 mmol), CuI (19 mg, 0.102 mmol) and triethylamine (0.71 mL, 5.08 mmol) were suspended in DMF (5 mL). The reaction mixture was degassed with bubbling N₂ then heated to 80° C. for 4 h. Following this period, the mixture was cooled to room temperature, diluted with EtOAc and washed with water, saturated aqueous NaHCO₃ and brine. The organic layer was dried over MgSO₄, filtered and concentrated. The crude material was purified by silica column chromatography (50% to 100% EtOAc/Hex) to provide the naphthyl alkyne (284 mg, 71%). A fraction of this material (123 mg, 0.156 mg) was dissolved in EtOH (4 mL) and treated with conc. HCl. The reaction mixture was stirred at reflux for 18 h. The solution was then concentrated. The resulting residue treated with 2-Methoxycarbonylamino-3-methyl-butyric acid (60 mg, 0.343 mmol) and HATU (130 mg, 0.343 mmol), suspended in DMF (3 mL) and cooled to 0° C. DIPEA (0.272 mL, 1.56 mmol) was added dropwise. After stirring for 4 h, NaOH (5M in H₂O, 0.300 mL, 1.5 mmol) was added. This mixture was stirred for 3 h then diluted with EtOAc and washed with 1 M LiOH (2×) then brine. The organic phase was dried over MgSO₄, filtered and concentrated. The crude residue was then purified by HPLC to afford the title compound (53 mg, 44%). MS (ESI) m/z 773 [M+H]⁺.

### Example 15

### Preparation of Compound 15

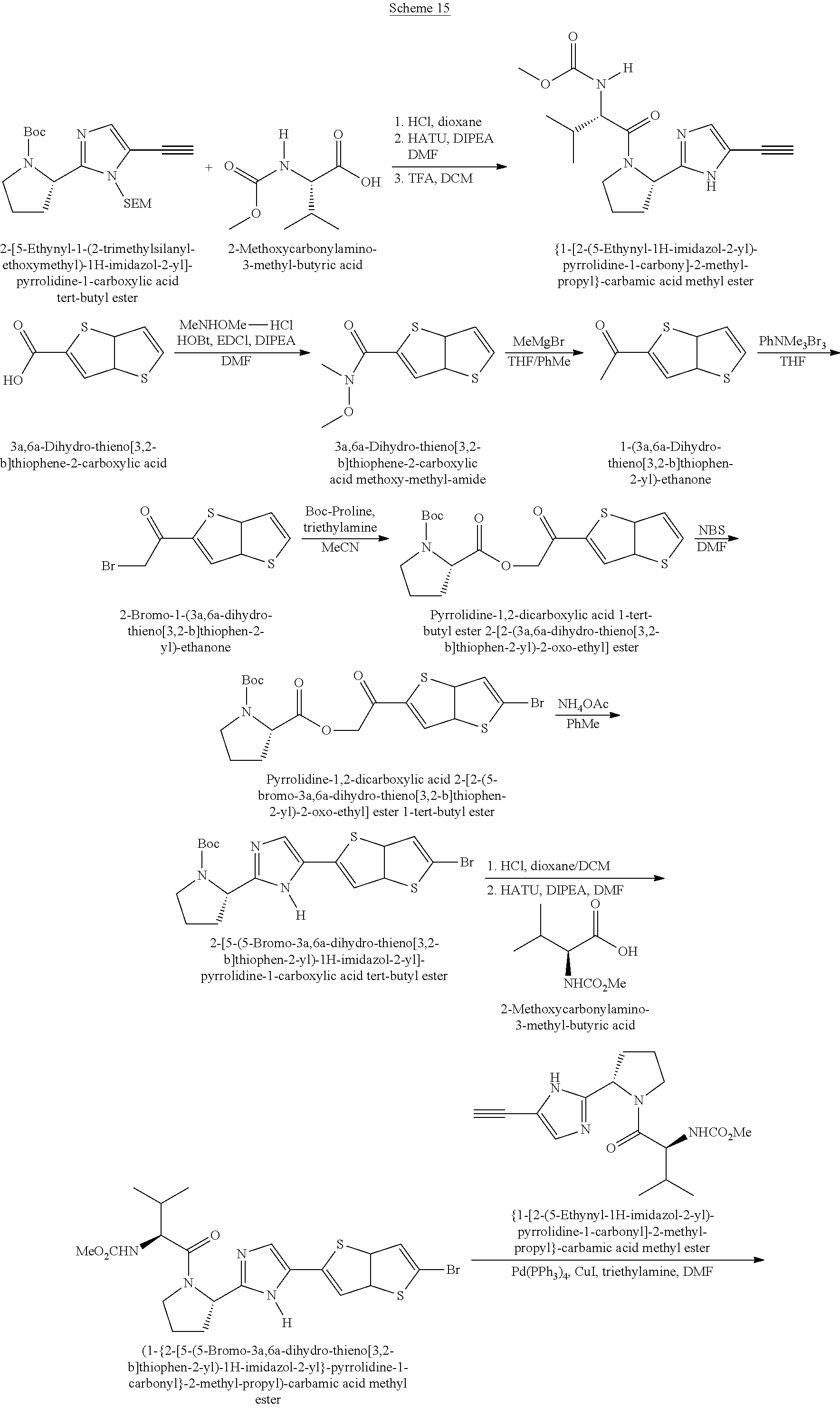

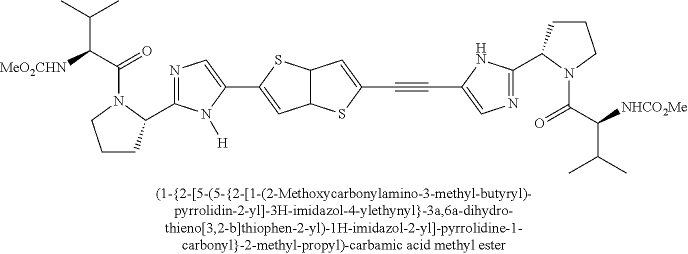

{1-[2-(5-Ethynyl-1H-imidazol-2-yl)-pyrrolidine-1-carbonyl]-2-methyl-propyl}-carbamic acid methyl ester: 2-[5-Ethynyl-1-(2-trimethylsilanyl-ethoxymethyl)-1H-imidazol-2-yl]-pyrrolidine-1-carboxylic acid tert-butyl ester (1.002 g, 2.56 mmol) was dissolved in dioxane (5 mL) and 4 M HCl in dioxane (5 mL) was added. The reaction mixture was stirred for 3 h and concentrated. To the residue was added 2-Methoxycarbonylamino-3-methyl-butyric acid (561 mg, 3.20 mmol), HATU (1.22 g, 3.20 mmol) and DMF (15 mL). The stirred reaction mixture was cooled to 0° C. and DIPEA (2.23 mL, 12.8 mmol) was added). After stirring for 3 h, the reaction mixture was diluted with ethyl acetate and washed with a saturated aqueous solution of sodium bicarbonate and brine. The combined organic layers were dried over magnesium sulfate and concentrated. The crude residue was purified by silica column chromatography (40% to 75% EtOAc/hexanes) to provide the coupled compound (741 mg, 65% over 2 steps). This material was dissolved in dichloromethane (10 mL) and trifluoroacetic acid (5 mL) was added. The stirred reaction mixture was heated to reflux for 4 h, then cooled to room temperature, and poured into a saturated aqueous solution of sodium bicarbonate. The aqueous phase was extracted 3 times with dichloromethane. The combined organic layers were dried over magnesium sulfate and concentrated. The crude residue was purified by silica column chromatography (0% to 10% MeOH/DMC) to provide {1-[2-(5-Ethynyl-1H-imidazol-2-yl)-pyrrolidine-1-carbonyl]-2-methyl-propyl}-carbamic acid methyl ester (525 mg, 100%).

3a,6a-Dihydro-thieno[3,2-b]thiophene-2-carboxylic acid methoxy-methyl-amide: 3a,6a-Dihydro-thieno[3,2-b]thiophene-2-carboxylic acid (2 g, 10.86 mmol) MeNHOMe-HCl (1.06 g, 10.86 mmol), HOBt (1.47 g, 10.86 mmol) and DIPEA (5.9 mL, 33.67 mmol) were combined in DMF (40 mL). To the stirred mixture was added EDCI (2.72 g, 14.12 mmol). After 5 h, EtOAc (100 mL) was added and the organics were washed with saturated aqueous NaHCO₃ and brine then dried over MgSO₄, filtered and concentrated. The crude residue was purified by silica column chromatography (20% to 45% EtOAc/Hex) to afford the title compound (1.98 g, 80%).

1-(3a,6a-Dihydro-thieno[3,2-b]thiophen-2-yl)-ethanone: 3a,6a-Dihydro-thieno[3,2-b]thiophene-2-carboxylic acid methoxy-methyl-amide (1.955 g, 8.60 mmol) was dissolved in THF. The stirred solution was cooled to 0° C. before methylmagnesium bromide (1.4 M in PhMe, 8.6 mL, 12.04 mmol) was added. The reaction was allowed to gradually warm to room temperature o/n, then it was quenched by addition of 10% HCl. The aqueous phase was extracted with diethyl ether. The organic phase was washed with brine then dried over MgSO₄, filtered and concentrated to afford the title compound (1.98 g, 80%).

2-Bromo-1-(3a,6a-dihydro-thieno[3,2-b]thiophen-2-yl)-ethanone: 1-(3a,6a-Dihydro-thieno[3,2-b]thiophen-2-yl)-ethanone (453 mg, 2.48 mmol) was dissolved in THF (12 mL) and phenyltrimethylammonium tribromide (932 mg, 2.48 mmol) was added. After stirring for 1 h, the suspension was filtered over Celite. The filtrate was diluted with diethyl ether, then washed with saturated aqueous NaHCO₃ and brine then dried over MgSO₄, filtered and concentrated to afford the title compound which was carried on without purification.

Pyrrolidine-1,2-dicarboxylic acid 1-tert-butyl ester 2-[2-(3a,6a-dihydro-thieno[3,2-b]thiophen-2-yl)-2-oxo-ethyl]ester: Crude 2-Bromo-1-(3a,6a-dihydro-thieno[3,2-b]thiophen-2-yl)-ethanone (2.48 mmol assuming complete conversion from starting material) was treated with Boc-proline and MeCN (25 mL). Triethylamine was added and the solution was stirred at room temperature for 1 h then concentrated. The crude residue was purified by silica column chromatography (14% to 35% EtOAc/Hex) to afford the title compound (595 mg, 61%).

Pyrrolidine-1,2-dicarboxylic acid 2-[2-(5-bromo-3a,6a-dihydro-thieno[3,2-b]thiophen-2-yl)-2-oxo-ethyl]ester 1-tert-butyl ester: Pyrrolidine-1,2-dicarboxylic acid 1-tert-butyl ester 2-[2-(3a,6a-dihydro-thieno[3,2-b]thiophen-2-yl)-2-oxo-ethyl]ester (595 mg, 1.5 mmol) was dissolved in DMF (7.5 mL) and treated with N-bromosuccinimide (295 mg, 1.65 mmol). The reaction mixture was stirred for 4 d at room temperature then diluted with EtOAc and washed with saturated aqueous NaHCO₃ and brine. The organic layer was dried over MgSO₄, filtered and concentrated. The crude residue was purified by silica column chromatography (20% to 50% EtOAc/Hex) to afford the title compound (469 mg, 66%).

2-[5-(5-Bromo-3a,6a-dihydro-thieno[3,2-b]thiophen-2-yl)-1H-imidazol-2-yl]-pyrrolidine-1-carboxylic acid tert-butyl ester: Pyrrolidine-1,2-dicarboxylic acid 2-[2-(5-bromo-3a,6a-dihydro-thieno[3,2-b]thiophen-2-yl)-2-oxo-ethyl]ester 1-tert-butyl ester (480 mg, 1.01 mmol) was treated with PhMe (10 mL) and ammonium acetate (1.56 g, 20.24 mmol). The reaction mixture was refluxed while stirring for 16 h, then cooled to room temperature. EtOAc was added and the organic phase was washed with saturated aqueous NaHCO₃ and brine. After it was dried over MgSO₄, it was filtered and concentrated. The crude residue was purified by silica column chromatography (25% to 60% EtOAc/Hex) to afford the title compound (378 mg, 82%).

(1-{2-[5-(5-Bromo-3a,6a-dihydro-thieno[3,2-b]thiophen-2-yl)-1H-imidazol-2-yl]-pyrrolidine-1-carbonyl}-2-methyl-propyl)-carbamic acid methyl ester: 2-[5-(5-Bromo-3a,6a-dihydro-thieno[3,2-b]thiophen-2-yl)-1H-imidazol-2-yl]-pyrrolidine-1-carboxylic acid tert-butyl ester (250 mg, 0.548 mmol) was dissolved in DCM (4 mL) and treated with HCl (4 M in dioxane, 1 mL, 4 mmol). After stirring for 1.5 h, the reaction mixture was concentrated. The solid was dried, then combined with 2-Methoxycarbonylamino-3-methyl-butyric acid (106 mg, 0.603 mmol), HATU (229 mg, 0.603 mmol) and DMF (6 mL). The stirred reaction mixture was cooled to 0° C. and DIPEA (0.48 mL, 2.74 mmol) was added dropwise. After 50 min, it was warmed to room temperature. 12 min later, the reaction mixture was diluted with EtOAc. The organic phase was washed with saturated aqueous NaHCO₃ and brine before being dried over MgSO₄, filtered and concentrated. The crude residue was purified by silica column chromatography to afford the title compound (252 mg, 90%).

(1-{2-[5-(5-{2-[1-(2-Methoxycarbonylamino-3-methyl-butyryl)-pyrrolidin-2-yl]-3H-imidazol-4-ylethynyl}-3a,6a-dihydro-thieno[3,2-b]thiophen-2-yl)-1H-imidazol-2-yl]-pyrrolidine-1-carbonyl}-2-methyl-propyl)-carbamic acid methyl ester: (1-{2-[5-(5-Bromo-3a,6a-dihydro-thieno[3,2-b]thiophen-2-yl)-1H-imidazol-2-yl]-pyrrolidine-1-carbonyl}-2-methyl-propyl)-carbamic acid methyl ester (140 mg, 0.440 mmol), {1-[2-(5-Ethynyl-1H-imidazol-2-yl)-pyrrolidine-1-carbonyl]-2-methyl-propyl}-carbamic acid methyl ester (130 mg, 0.254 mmol), Pd(PPh₃)₄ (29 mg, 0.0254 mmol), CuI (10 mg, 0.0508 mmol) and triethylamine (0.354 mmol, 2.54 mmol) were combined in DMF (2.5 mL) and degassed with N₂ for 17 min. The reaction was heated to 85° C. for 4 h then cooled to room temperature, diluted with EtOAc and washed with saturated aqueous NaHCO₃ (2×) and brine. The organic layer was dried over MgSO₄, filtered and concentrated. The crude residue was purified by HPLC chromatography to afford the title compound (34 mg, 18%). MS (ESI) m/z 749 [M+H]⁺.

### Example 16

### Preparation of Compound 16

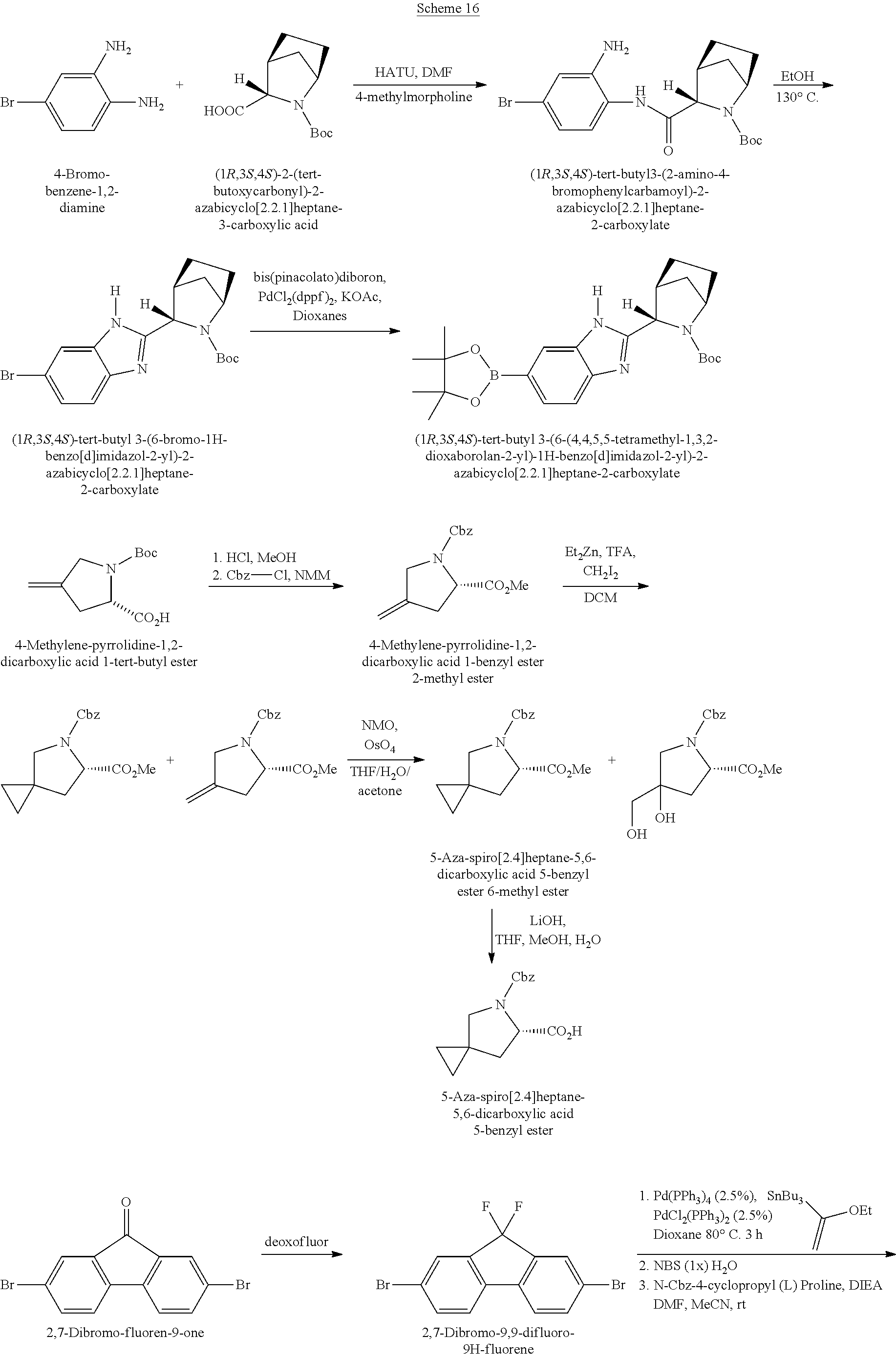

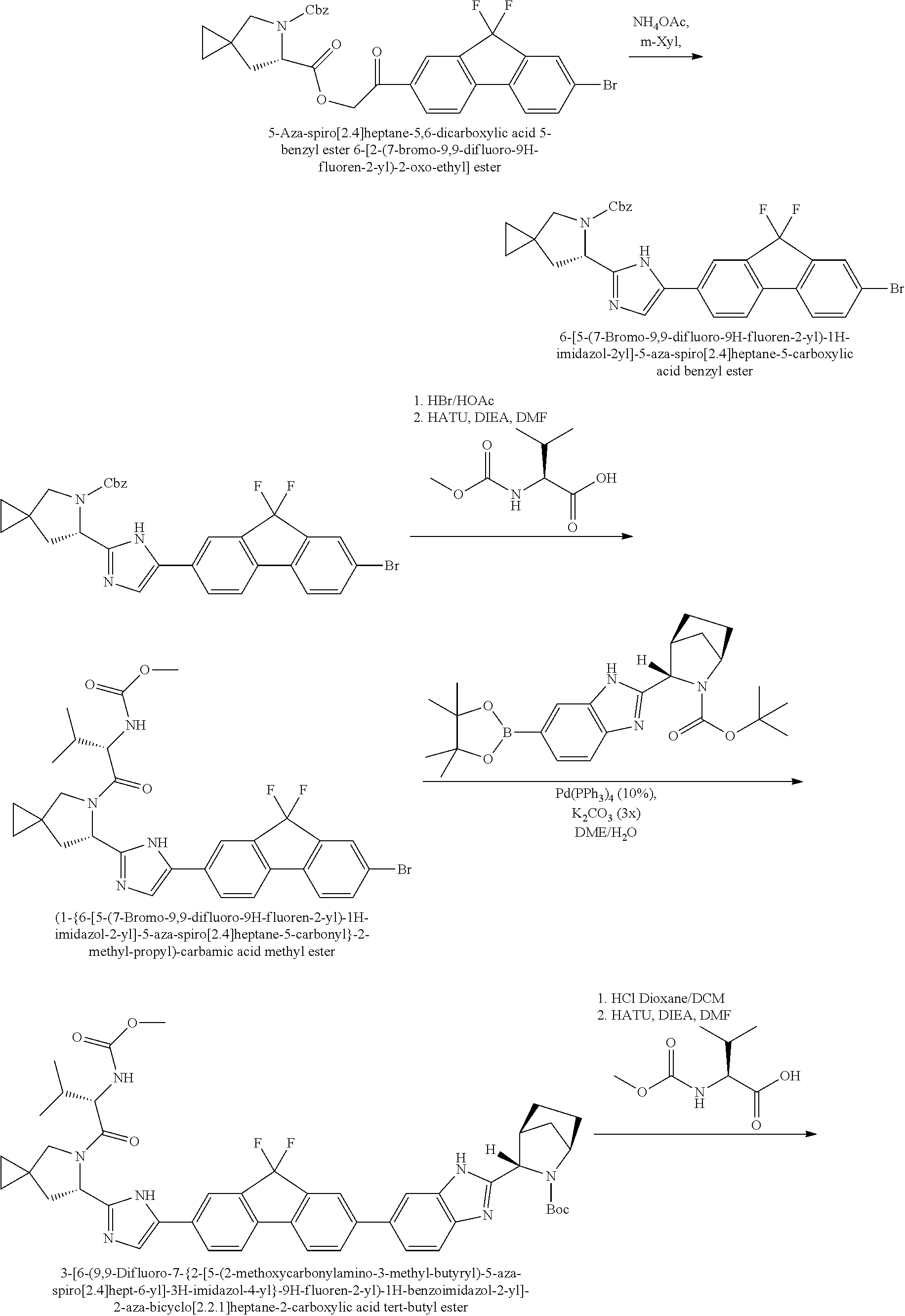

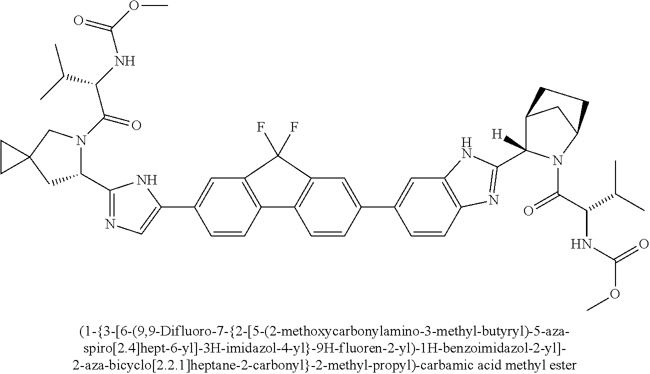

(1R,3S,4S)-tert-butyl 3-(2-amino-4-bromophenylcarbamoyl)-2-azabicyclo[2.2.1]heptane-2-carboxylate: To a solution of (1R,3S,4S)-2-(tert-butoxycarbonyl)-2-azabicyclo[2.2.1]heptane-3-carboxylic acid (0.327 g, 1.36 mmol, 1 eq.), 4-Bromo-benzene-1,2-diamine (0.507 g, 2.71 mmol, 2 eq.) and 4-methylmorpholine (0.299 mL, 2 eq.) in 10 mL DMF was added HATU (0.543 g, 1.05 eq.). The reaction mixture was stirred at room temperature for 1 hour then concentrated down. The reaction mixture was diluted with ethyl acetate and washed with diluted NaHCO₃ aqueous solution and brine. The organic layer was concentrated down and purified by flash column chromatography (silica gel, 20 to 80% ethyl acetate/hexane) to give a mixture of regioisomer (1R,3S,4S)-tert-butyl 3-(2-amino-4-bromophenylcarbamoyl)-2-azabicyclo[2.2.1]heptane-2-carboxylate.

(1R,3S,4S)-tert-butyl 3-(6-bromo-1H-benzo[d]imidazol-2-yl)-2-azabicyclo[2.2.1]heptane-2-carboxylate: The above mixture of regioisomer (1R,3S,4S)-tert-butyl 3-(2-amino-4-bromophenylcarbamoyl)-2-azabicyclo[2.2.1]heptane-2-carboxylate was dissolved in ethanol and heated to 130° C. in sealed tube overnight and continue heating at 170° C. for 3 days. LC-MS showed desired product and Boc cleaved product (about 1:1 ratio). The mixture was concentrated down and dissolved DCM. Di-tert-butyl dicarbonate (0.6 eq.) was added and reaction was stirred overnight at room temperature. The reaction mixture was concentrated down and purified by flash column chromatography (silica gel, 20 to 80% ethyl acetate/hexane) to give (1R,3S,4S)-tert-butyl 3-(6-bromo-1H-benzo[d]imidazol-2-yl)-2-azabicyclo[2.2.1]heptane-2-carboxylate (0.383 g, 72%) as an orange foam.

(1R,3S,4S)-tert-butyl 3-(6-(4,4,5,5-tetramethyl-1,3,2-dioxaborolan-2-yl)-1H-benzo[d]imidazol-2-yl)-2-azabicyclo[2.2.1]heptane-2-carboxylate: A solution of (1R,3S,4S)-tert-butyl 3-(6-bromo-1H-benzo[d]imidazol-2-yl)-2-azabicyclo[2.2.1]heptane-2-carboxylate, bis(pinacolato)diboron, tetrakis(triphenylphosphine)palladium, and potassium acetate in 1,4-dioxanes was heated at 80° C. overnight. Following standard workup procedures, the crude material was purified by silica gel chromatography to afford the desired (1R,3S,4S)-tert-butyl 3-(6-(4,4,5,5-tetramethyl-1,3,2-dioxaborolan-2-yl)-1H-benzo[d]imidazol-2-yl)-2-azabicyclo[2.2.1]heptane-2-carboxylate.

4-Methylene-pyrrolidine-1,2-dicarboxylic acid 1-benzyl ester 2-methyl ester: 4-Methylene-pyrrolidine-1,2-dicarboxylic acid 1-tert-butyl ester (10.0 g, 44 mmol) was dissolved in MeOH (75 mL) at room temperature and HCl (4M in dioxane, 75 mL) was added. Stirring at room temperature was continued for 4 hours. All volatiles were removed in vacuo and a beige solid was obtained.

The crude material was suspended in DCM (100 mL) and N-Methyl morpholine (13.3 g, 132 mmol) was added. The mixture was cooled to 0° C. and benzyl chloroformate (8.26 g, 48.4 mmol) was added while stirring. After 30 minutes, the reaction was warmed to room temperature and the solution was washed with water and aqueous HCl (1M). The solution was dried over sodium sulfate. Filtration and evaporation of solvents gave crude product, which was purified by silica gel chromatography (eluent: EtOAc/hexanes) to yield the product (10.2 g). LCMS-ESI⁺: calc'd for C₁₅H₁₇NO₄: 275.3 (M⁺); Found: 276.4 (M+H⁺). An oven-dried 3-neck round bottom flask was equipped with a nitrogen inlet adaptor and a 250 mL addition funnel. The third neck was sealed with a septum. The flask was charged with a stir bar, dichlorormethane (120 mL) and diethyl zinc (1.0 M in hexane, 118 mL, 118 mmol) then cooled to 0° C. in an ice bath. The addition funned was charged with dichloromethane (40 mL) and trifluoroacetic acid (9.1 mL, 118 mmol). After the diethyl zinc solution had cooled to 0° C. (about 25 minutes), the trifluoroacetic acid solution was added dropwise over 20 min to the stirred reaction mixture. After stirring for another 20 min at 0° C., diiodomethane (9.5 mL, 118 mmol) was added slowly over 4 min. After another 20 min, 4-methylene-pyrrolidine-1,2-dicarboxylic acid 1-benzyl ester 2-methyl ester (8.10 g, 29.4 mmol) was added in 30 mL dichloromethane by cannula. The flask containing 4-methylene-pyrrolidine-1,2-dicarboxylic acid 1-benzyl ester 2-methyl ester was then rinsed with another 10 mL dichloromethane and this solution was also transferred to the reaction mixture by cannula. The reaction mixture was allowed to warm to room temperature and stirred for 110 h (about 5 days) after which the reagents were quenched with saturated aqueous ammonium chloride (˜150 mL). The contents of the flask were slowly poured into a 2 L sep funnel containing saturated aqueous sodium bicarbonate (˜800 mL). The aqueous phase was extracted three times with 300 mL ethyl acetate. The combined organics were dried over magnesium sulfate and concentrated to provide the crude material. The crude material was dissolved in 3:1:1 THF/water/acetone (165 mL) then treated with N-methylmorpholine-N-oxide (3.45 g, 29.4 mmol) and osmium tetroxide (4 wt % in water, 5 mL, 0.818 mmol). After stirring at room temperature for 7 h, the reagents were quenched with 1 M aqueous sodium thiosulfate (˜100 mL). The contents of the flask were then poured into a 1 L sep funnel containing water (˜300 mL). The aqueous phase was extracted three times with 300 mL dichloromethane. The combined organics were dried over magnesium sulfate and concentrated. The crude residue was purified by silica column chromatography (5% to 45% EtOAc/hexane) to provide 5-aza-spiro[2.4]heptane-5,6-dicarboxylic acid 5-benzyl ester 6-methyl ester as a clear oil (5.54 g, 19.15 mmol, 65%) as a clear oil. ¹H NMR (CDCl₃) δ 7.36-7.29 (m, 5H), 5.21-5.04 (m, 2H), 4.56-4.47 (m, 1H), 3.75 (s, 1.5H), 3.60 (m, 1.5H), 03.51-3.37 (m, 2H), 2.32-2.25 (m, 1H), 1.87-1.80 (m, 1H), 0.64-0.51 (m, 4H).

5-Aza-spiro[2.4]heptane-5,6-dicarboxylic acid 5-benzyl ester:

5-Aza-spiro[2.4]heptane-5,6-dicarboxylic acid 5-benzyl ester 6-methyl ester (244 mg, 0.840 mmol) was dissolved in THF (2.0 mL)/MeOH (1.5 mL). An aqueous solution of LiOH (35.5 mg, 0.84 mmol) was added and stirring at room temperature was continued. After 3 hours, the reaction was neutralized with aqueous HCl (1M) and the organic solvents were removed in vacuo. The crude mixture was diluted with water and EtOAc and the organic layer was collected. All volatiles were removed in vacuo and the crude acid was used without further purification. LCMS-ESI⁺: calc'd for C₁₆H₁₇NO₄: 275.3 (M⁺); Found: 276.3 (M+H⁺).

2,7-Dibromo-9,9-difluoro-9H-fluorene: 2,7-Dibromo-fluoren-9-one (4.0 g, 11.8 mmol) was suspended in deoxofluor (12 mL) at room temperature and EtOH (4 drops) was added. The stirred suspension was heated at T=90° C. for 24 hours (CAUTION: Use of deoxofluor at elevated temperatures, as described above, is cautioned as rapid and violent exotherms may occur). The reaction was cooled to room temperature and poured onto ice containing sodium bicarbonate. A solid formed and was collected via filtration. The crude material was taken into EtOAc and was washed with aqueous HCl (1M) and brine. The solution was dried over sodium sulfate. Filtration and evaporation of solvents gave crude product, which was purified by silica gel chromatography (eluent: EtOAc/hexanes) to yield the product (3.2 g).

¹⁹F-NMR: 282 MHz, (dmso-d₆) δ: −111.6 ppm.

Before using the material in the next step, it was exposed as a solution in EtOAc to charcoal.

5-Aza-spiro[2.4]heptane-5,6-dicarboxylic acid 5-benzyl ester 6-[2-(7-bromo-9,9-difluoro-9H-fluoren-2-yl)-2-oxo-ethyl]ester: 2,7-Dibromo-9,9-difluoro-9H-fluorene (372 mg, 1.04 mmol), Pd(PPh₃)₄ (30.0 mg, 0.026 mmol), PdCl₂(PPh₃)₂ (18.2 mg, 0.026 mmol), As(PPh₃)₃ (5.0 mg) were dissolved in dioxane (10 mL) under an argon atmosphere. Ethoxyvinyl-tributyl tin (376.4 mg, 1.04 mmol) was added. The mixture was heated for 140 minutes at 85° C. (oil bath). The reaction was cooled to room temperature. N-bromo succinimide (177 mg, 1.0 mmol) was added followed by water (2 mL). The reaction was stirred at room temperature for 3 hours, after which the majority of the dioxane was removed in vacuo. The crude reaction mixture was diluted with EtOAc and was washed with water. All volatiles were removed in vacuo. Toluene was added and all volatiles were removed in vacuo for a second time. The crude material was dissolved in DMF/MeCN (2 mL, 1:1) at room temperature. A solution of N-Cbz-4-cyclopropyl (L) Proline (0.84 mmol) and DIEA (268 mg, 2.08 mmol) in MeCN (2 mL) was added and stirring at room temperature was continued. After 14 hours, most of the MeCN was removed in vacuo and the crude reaction mixture was diluted with EtOAc. The mixture was washed with aqueous HCl (1M), aqueous LiCl solution (5%), brine, and was dried over sodium sulfate. Filtration and evaporation of solvents gave the crude reaction product, which was purified via silica gel chromatography (eluent: EtOAc/hexanes) to yield the product (176 mg). LCMS-ESI⁺: calc'd for C₃₀H₂₄BrF₂NO₅: 596.4 (M⁺); Found: 595.2/597.2 (M+H⁺).

6-[5-(7-Bromo-9,9-difluoro-9H-fluoren-2-yl)-1H-imidazol-2-yl]-5-aza-spiro[2.4]heptane-5-carboxylic acid benzyl ester: 5-Aza-spiro[2.4]heptane-5,6-dicarboxylic acid 5-benzyl ester 6-[2-(7-bromo-9,9-difluoro-9H-fluoren-2-yl)-2-oxo-ethyl]ester (172 mg, 0.293 mmol) was dissolved in m-xylenes (6.0 mL). Ammonium acetate (226 mg, 2.93 mmol) was added and the reaction was stirred at 140° C. for 60 minutes under microwave conditions. The reaction was cooled to room temperature and all volatiles were removed in vacuo. The crude material was purified via silica gel chromatography (eluent: EtOAc/hexanes) to yield the product (80.3 mg).

LCMS-ESI⁺: calc'd for C₃₀H₂₄BrF₂N₃O₂: 576.4 (M⁺); Found: 575.2/577.2 (M+H⁺).

(1-{6-[5-(7-Bromo-9,9-difluoro-9H-fluoren-2-yl)-1H-imidazol-2-yl]-5-aza-spiro[2.4]heptane-5-carbonyl}-2-methyl-propyl)-carbamic acid methyl ester: 6-[5-(7-Bromo-9,9-difluoro-9H-fluoren-2-yl)-1H-imidazol-2-yl]-5-aza-spiro[2.4]heptane-5-carboxylic acid benzyl ester (800 mg, 1.38 mmol) was dissolved in DCM (15 mL) and HBr in AcOH (37%, 2 mL) was added and stirring at room temperature was continued. After 180 minutes, the suspension was diluted with hexanes and the solid was collected via filtration and was washed with hexanes and subjected to vacuum. The crude material was used in the next step without further purification. The crude material was dissolved in DMF (4.0 mL) and DIEA (356 mg, 2.76 mmol) was added. A solution of 2-(L)-Methoxycarbonylamino-3-methyl-butyric acid (242 mg, 1.38 mmol), HATU (524 mg, 1.38 mmol) and DIEA (178 mg, 1.38 mmol) in DMF (1 mL) was added. The reaction was stirred at room temperature. After 50 minutes, the reaction was diluted with EtOAc and was washed with aqueous bicarbonate solution, aqueous LiCl solution (5%), brine, and was dried over sodium sulfate. Filtration and removal of solvents in vacuo gave the crude material, which was purified by silica gel chromatography (eluent: EtOAc/hexanes) to yield the slightly impure product (878 mg). LCMS-ESI⁺: calc'd for C₂₉H₂₉BrF₂N₄O₃: 599.5 (M⁺); Found: 598.5/600.5 (M+H⁺).

3-[6-(9,9-Difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carboxylic acid tert-butyl ester: (1-{6-[5-(7-Bromo-9,9-difluoro-9H-fluoren-2-yl)-1H-imidazol-2-yl]-5-aza-spiro[2.4]heptane-5-carbonyl}-2-methyl-propyl)-carbamic acid methyl ester (840 mg, 1.4 mmol), 3-[6-(4,4,5,5-Tetramethyl-[1,3,2]dioxaborolan-2-yl)-1H-benzoimidazol-2-yl]-2-aza-bicyclo[2.2.1]heptane-2-carboxylic acid tert-butyl ester (615 mg, 1.4 mmol), Pd(PPh₃)₄ (161 mg, 0.14 mmol), K₂CO₃ (579 mg, 4.2 mmol), were dissolved in DME (15 mL)/water (3 mL) under an argon atmosphere. The mixture was heated for 120 minutes at 85-90° C. (oil bath). After 120 minutes additional boronate ester (61 mg, 0.14 mmol) was added and heating was continued. After 3 hours, the reaction was cooled to room temperature. Most of the DME was removed in vacuo and the crude reaction mixture was diluted with EtOAc. The mixture was washed with brine and was dried over sodium sulfate. Filtration and evaporation of solvents gave the crude reaction product, which was purified via silica gel chromatography (eluent: EtOAc/hexanes) to yield the product (878 mg). LCMS-ESI⁺: calc'd for C₄₇H₅₁F₂N₇O₅: 831.9 (M⁺); Found: 832.7 (M+H⁺).

(1-{3-[6-(9,9-Difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methyl-propyl)-carbamic acid methyl ester (Example ED): 3-[6-(9,9-Difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carboxylic acid tert-butyl ester (115 mg, 0.138 mmol) was dissolved in DCM (2 mL) and HCl in dioxane (4M, 2 mL) was added and stirring at room temperature was continued. After 20 minutes, all volatiles were removed in vacuo. The crude material was used in the next step without further purification. The crude material was dissolved in DMF (1.5 mL) and DIEA (53.4 mg, 0.414 mmol) was added. A solution of 2-(L) Methoxycarbonylamino-3-methyl-butyric acid (24.2 mg, 0.138 mmol), HATU (52.4 mg, 0.138 mmol) and DIEA (17.8 mg, 0.138 mmol) in DMF (1 mL) was added. The reaction was stirred at room temperature. After 20 minutes, the reaction was diluted with EtOAc and was washed with aqueous bicarbonate solution, aqueous LiCl solution (5%), brine, and was dried over sodium sulfate. Filtration and removal of solvents in vacuo gave the crude material, which was purified by RP-HPLC (eluent: water/MeCN w/0.1% TFA) to yield the product (76 mg). LCMS-ESI⁺: calc'd for C₄₉H₅₄F₂N₈O₆: 888.9 (M⁺); Found: 890.0 (M+H⁺).

¹H-NMR: 300 MHz, (dmso-d₆) δ: 8.20-7.99 (m, 8H), 7.73 (s, 2H), 7.37-7.27 (m, 2H), 5.25 (dd, J=7.2 Hz, 1H), 4.78 (s, 1H) 4.54 (s, 1H), 4.16 (m, 1H), 4.02 (m, 1H), 3.87 (m,1H), 3.74 (m, 1H), 3.55 (s, 3H), 3.53 (s, 3H), 2.75 (m, 1H), 2.25 (m, 2H), 2.09-2.04 (m, 2H), 1.88-1.79 (m, 2H), 1.54 (m, 1H), 0.94-0.77 (m, 15H) 0.63 (m, 4H) ppm. ¹⁹F-NMR: 282 MHz, (dmso-d₆) δ: −109.1 ppm [−74.8 ppm TFA].

### Example 17

### Preparation of Compound 17

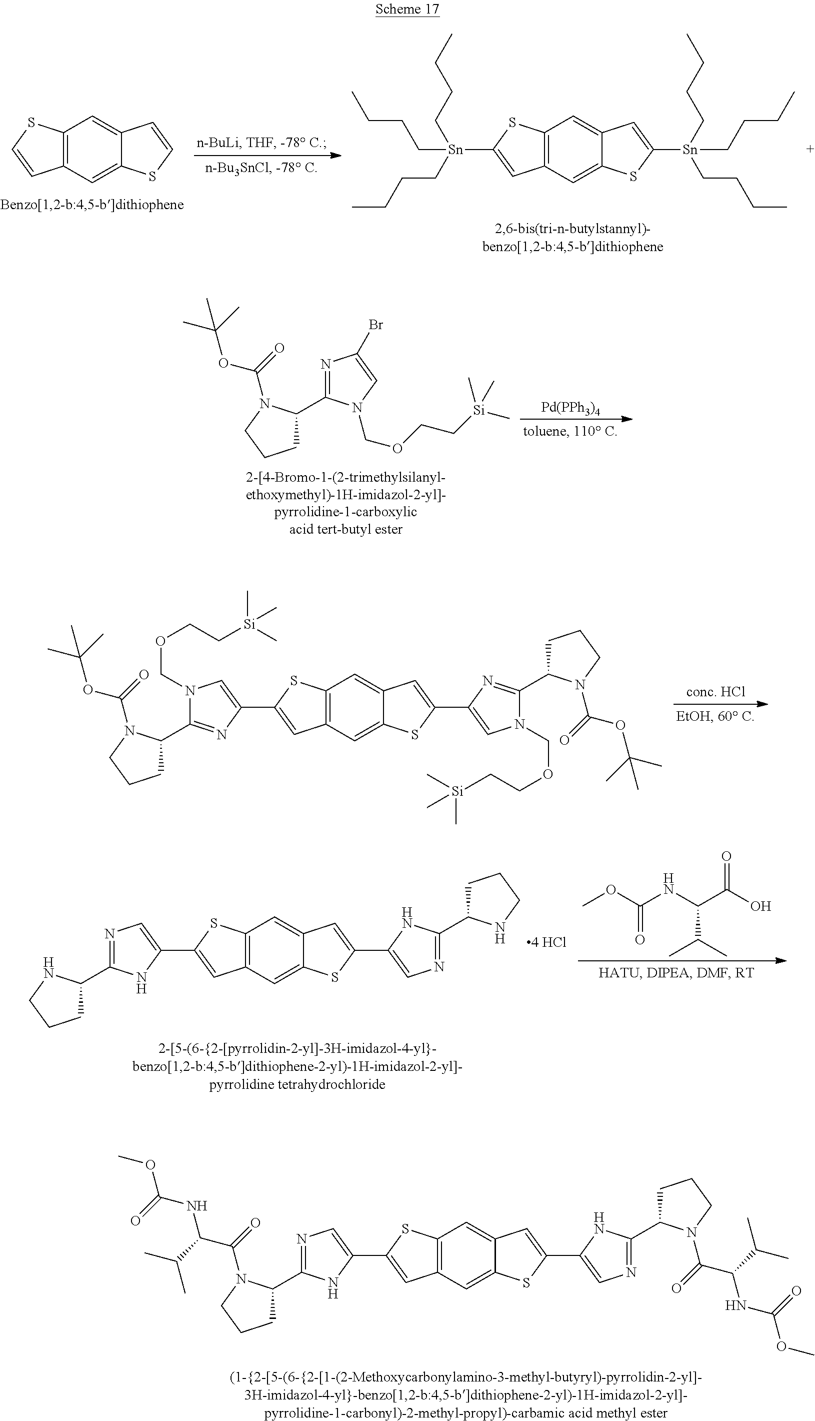

2,6-Bis(tri-n-butylstannyl)-benzo[1,2-b:4,5-b′]dithiophene: To a stirred solution of benzo[1,2-b:4,5-b′]dithiophene (820 mg, 4.3 mmol) in THF (100 mL) under argon at −78° C. was added a solution of n-butyllithium (2.5 M, 3.44 mL, 8.6 mmol). The solution was stirred at −78° C. for 30 minutes and then warmed to −20° C. for 30 minutes. Tri-n-butyltin chloride (2.34 mL, 8.6 mmol) was added and the reaction mixture was stirred at −20° C. for 30 minutes and then allowed to warm to room temperature. After 16 hours, hexane was added and the reaction was successively washed with water and brine, dried (MgSO₄), concentrated and purified by flash chromatography (100% hexanes). 2,6-bis(tri-n-butylstannyl)-benzo[1,2-b:4,5-b′]dithiophene (1.4 g, 42%) was isolated along with product contaminated with the monostannylated benzodithiophene. ¹H-NMR: 400 MHz, (CDCl₃) δ: 8.27 (s, 2H), 7.38 (s, 2H), 1.65-1.57 (m, 12H), 1.41-1.32 (m, 12H), 1.26-1.11 (m, 12H), 0.91 (t, J=7.3 Hz, 18H) ppm.

Fully protected 2-[5-(6-{2-[pyrrolidin-2-yl]-3H-imidazol-4-yl}-benzo[1,2-b:4,5-b′]dithiophene-2-yl)-1H-imidazol-2-yl]-pyrrolidine: Pd(PPh₃)₄ (61 mg, 0.053 mmol) was added to a degassed solution of 2,6-bis(tri-n-butylstannyl)-benzo[1,2-b:4,5-b′]dithiophene (202 mg, 0.26 mmol) and 2-[4-Bromo-1-(2-trimethylsilanyl-ethoxymethyl)-1H-imidazol-2-yl]-pyrrolidine-1-carboxylic acid tert-butyl ester (260 mg, 0.58 mmol) in toluene (4 mL). The reaction was refluxed for 24 hours, then cooled to room temperature and filtered through Celite and a palladium scavenging column (Stratospheres™ PL-Guanidine MP SPE+, Part #: PL3514-CM89). The solids were rinsed twice with toluene. The filtrate was concentrated and the crude product purified by flash chromatography to yield the desired, fully protected product (100 mg, 41%). LCMS-ESI⁺: calculated for C₄₆H₆₈N₆O₆S₂Si₂: 920.42; observed [M+1]⁺: 921.45.

(1-{2-[5-(6-{2-[1-(2-Methoxycarbonylamino-3-methyl-butyryl)-pyrrolidin-2-yl]-3H-imidazol-4-yl}-benzo[1,2-b:4,5-b′]dithiophene-2-yl)-1H-imidazol-2-yl]-pyrrolidine-1-carbonyl}-2-methyl-propyl)-carbamic acid methyl ester: A solution of fully protected 2-[5-(6-{2-[pyrrolidin-2-yl]-3H-imidazol-4-yl}-benzo[1,2-b:4,5-b′]dithiophene-2-yl)-1H-imidazol-2-yl]-pyrrolidine (100 mg, 0.11 mmol), ethanol (4 mL) and concentrated HCl (1 mL) was heated to 60° C. for 16 hours. The reaction was concentrated and the crude material dissolved in DCM (10 mL). This solution was concentrated to yield crude 2-[5-(6-{2-[pyrrolidin-2-yl]-3H-imidazol-4-yl}-benzo[1,2-b:4,5-b′]dithiophene-2-yl)-1H-imidazol-2-yl]-pyrrolidine tetrahydrochloride. To this material was added a solution of 2-methoxycarbonylamino-3-methylbutyric acid (38 mg, 0.22 mmol) and HATU (83 mg, 0.22 mmol) in DMF (1.5 mL). To the resulting solution was added diisopropylethylamine (190 μL, 1.1 mmol). After stirring for 2 hours at room temperature, the reaction was concentrated and purified twice by preparative reverse phase HPLC (Gemini, 10 to 45% ACN/H₂O+0.1% HCO₂H). The product fractions were passed through a freebasing column (Stratospheres™ PL-HCO₃MP SPE, Part #: PL3540-C603) and lyophilized to give (1-{2-[5-(6-{2-[1-(2-methoxycarbonylamino-3-methyl-butyryl)-pyrrolidin-2-yl]-3H-imidazol-4-yl}-benzo[1,2-b:4,5-b′]dithiophene-2-yl)-1H-imidazol-2-yl]-pyrrolidine-1-carbonyl}-2-methyl-propyl)-carbamic acid methyl ester (29 mg, 34%). LCMS-ESI⁺: calculated for C₃₈H₄₆N₈O₆S₂: 774.95; observed [M+1]⁺: 775.96. ¹H-NMR: 400 MHz, (CD_{OD) δ:} 8.16-8.11 (m, 2H), 7.49-7.47 (m, 2H), 7.38-7.29 (m, 2H), 5.18-5.15 (m, 2H), 4.24 (d, J=7.4 Hz, 2H), 4.04-3.96 (m, 2H), 3.91-3.86 (m, 2H), 3.66 (br s, 6H), 2.38-2.17 (m, 6H), 2.11-1.98 (m, 4H), 1.00-0.89 (m, 12H) ppm.

### BIOLOGICAL EXAMPLES

### Assay Protocol

### High Throughput Replicon Assay (HTBS)

Replicon cells harboring H77 (genotype 1a) or Con1 (genotype 1b) HCV RNA and Renilla luciferase reporter were seeded in 384-well black plates at a density of 1.6×103 cells per well in 90 μl of DMEM culture medium, excluding G-418. Compounds were serially diluted in 100% DMSO and added to cells at a 1:225 dilution, achieving a final concentration of 0.44% DMSO in a total volume of 90 μL with a Biotek μFlow Workstation. Cell plates were incubated at 37° C. with 5% CO2 for 3 days, after which culture media were removed and cells were assayed for luciferase activity as a marker for replication level. Luciferase expression was measured using Dual-Glo luciferase assay reagents (Promega, Madison, Wis.). Briefly, 20 μL of Dual-Glo luciferase buffer was added to lyse the cells for 10 min and subsequently 20 μL of a diluted Dual-Glo Stop & Glo substrate (1:100) was added to each well. Luminescence signal was measured on a Perkin Elmer Envision Plate Reader after incubation for 10 minutes. Luciferase levels were converted into percentages relative to the untreated controls (defined as 100%) and data were fit to the logistic dose response equation y=a/(1+(x/b)c) using XLFit4 software (IDBS, Emeryville, Calif.). EC₅₀ values were calculated from the resulting equations. Alternatively, antiviral activity may be analyzed by HCV NS3 Protease IC₅₀ Determination. HCV NS3 protease activity was monitored using a fluorescence resonance energy transfer (FRET) depsipeptide substrate (RET S1, Anaspec, San Jose, Calif.) based on the method of Taliani, Taliani M, Bianchi E, Narjes F, Fossatelli M, Urbani A, Steinkuhler C, et al. A continuous assay of hepatitis C virus protease based on resonance energy transfer depsipeptide substrates. Anal Biochem 1996; 240 (1):60-7, herein incorporated by reference with regard to performing such assay.

Briefly, 2-10 nM of purified NS3 protease domains were pre-incubated at 37° C. for 10 minutes with 20 μM isogenic NS4A peptide cofactors (Sigma, St. Louis, Mo.), in 40% glycerol buffer with 50 mM HEPES pH 7.5 and 10 mM DTT. Compounds were diluted serially 1:3 in DMSO, incubated with the enzyme/cofactor mixture for 10 minutes and reactions were started by the addition of 2 μM RET S1 substrate (final concentration). Fluorescence increase was measured continuously over one hour using a Victor3 V fluorescence plate reader (Perkin Elmer, Waltham, Mass.). Initial velocities were calculated for each inhibitor concentration using Workout 1.5 software (DAZDAQ, East Sussex, UK) with the maximal slope algorithm. Velocity data were converted into percentages relative to the untreated control (defined as 100%) and non-linear regression was performed to calculate 50% inhibitory concentrations (IC₅₀ values).

NS3 Enzymatic Potency: Purified NS3 protease is complexed with NS4A peptide and then incubated with serial dilutions of the compounds (DMSO used as solvent). Reactions are started by addition of dual-labeled peptide substrate and the resulting kinetic increase in fluorescence is measured. Non-linear regression of velocity data is performed to calculate IC₅₀s. Activity is initially tested against genotype 1b protease. Depending on the potency obtained against genotype 1b, additional genotypes (1a, 2a, 3) and or protease inhibitor resistant enzymes (D168Y, D168V, or A156T mutants) may be tested. BILN-2061 is used as a control during all assays. Compounds of the Examples were evaluated in this assay and were found to have IC₅₀ values of less than about 1 μM.

Replicon Potency and Cytotoxicity: Huh-luc cells (stably replicating Bartenschlager's 1389luc-ubi-neo/NS3-3′/ET genotype 1b replicon) is treated with serial dilutions of compound (DMSO is used as solvent) for 72 hours. Replicon copy number is measured by bioluminescence and non-linear regression is performed to calculate EC₅₀s. Parallel plates treated with the same drug dilutions are assayed for cytotoxicity using the Promega CellTiter-Glo cell viability assay. Depending on the potency achieved against the 1b replicon, compounds may be tested against a genotype 1a replicon and/or inhibitor resistant replicons encoding D168Y or A156T mutations. BILN-2061 is used as a control during all assays. Compounds of the Examples were evaluated in this assay and were found to have EC₅₀ values of less than about 5 μM.

### Effect of Serum Proteins on Replicon Potency

Replicon assays are conducted in normal cell culture medium (DMEM+10% FBS) supplemented with physiologic concentrations of human serum albumin (40 mg/mL) or α-acid glycoprotein (1 mg/mL). EC₅₀s in the presence of human serum proteins are compared to the EC₅₀ in normal medium to determine the fold shift in potency.

Enzymatic Selectivity: The inhibition of mammalian proteases including Porcine Pancreatic Elastase, Human Leukocyte Elastase, Protease 3, and Cathepsin D are measured at Kₘ for the respective substrates for each enzyme. IC₅₀ for each enzyme is compared to the IC₅₀ obtained with NS3 1b protease to calculate selectivity.

MT-4 Cell Cytotoxicity: MT4 cells are treated with serial dilutions of compounds for a five day period. Cell viability is measured at the end of the treatment period using the Promega CellTiter-Glo assay and non-linear regression is performed to calculate CC₅₀.

Compound Concentration Associated with Cells at EC₅₀: Huh-luc cultures are incubated with compound at concentrations equal to EC₅₀. At multiple time points (0-72 hours), cells are washed 2× with cold medium and extracted with 85% acetonitrile; a sample of the media at each time-point is also extracted. Cell and media extracts are analyzed by LC/MS/MS to determine the molar concentration of compounds in each fraction

Solubility and Stability: Solubility is determined by taking an aliquot of 10 mM DMSO stock solution and preparing the compound at a final concentration of 100 μM in the test media solutions (PBS, pH 7.4 and 0.1 N HCl, pH 1.5) with a total DMSO concentration of 1%. The test media solutions are incubated at room temperature with shaking for 1 hr. The solutions are then centrifuged and the recovered supernatants are assayed on the HPLC/UV. Solubility can be calculated by comparing the amount of compound detected in the defined test solution compared to the amount detected in DMSO at the same concentration. The stability of compounds after 1 hour incubation in the test media at 37° C. is also determined.

Stability in Cryo-preserved Human, Dog, and Rat Hepatocytes: Each compound is incubated for up to 1 hour in hepatocyte suspensions (100 μl, 80,000 cells per well) at 37° C. Cryopreserved hepatocytes are reconstituted in the serum-free incubation medium. The suspension is transferred into 96-well plates (50 μL/well). The compounds are diluted to 2 μM in incubation medium and then are added to hepatocyte suspensions to start the incubation. Samples are taken at 0, 10, 30 and 60 minutes after the start of incubation and reaction can be quenched with a mixture consisting of 0.3% formic acid in 90% acetonitrile/10% water. The concentration of the compound in each sample is analyzed using LC/MS/MS. The disappearance half-life of the compound in hepatocyte suspension is determined by fitting the concentration-time data with a monophasic exponential equation. The data is also scaled up to represent intrinsic hepatic clearance and/or total hepatic clearance.

Stability in Hepatic S9 Fraction from Human, Dog, and Rat: Each compound is incubated for up to 1 hour in S9 suspension (500 μl, 3 mg protein/mL) at 37° C. (n=3). The compounds are added to the S9 suspension to start the incubation. Samples are taken at 0, 10, 30, and 60 minutes after the start of incubation. The concentration of the compound in each sample is analyzed using LC/MS/MS. The disappearance half-life of the compound in S9 suspension is determined by fitting the concentration-time data with a monophasic exponential equation.

Caco-2 Permeability: Both forward (A-to-B) and reverse (B-to-A) permeability is measured. Caco-2 monolayers are grown to confluence on collagen-coated, microporous, polycarbonate membranes in 12-well Costar Transwell® plates. The compounds are dosed on the apical side for forward permeability (A-to-B), and are dosed on the basolateral side for reverse permeability (B-to-A). The cells are incubated at 37° C. with 5% CO₂ in a humidified incubator. At the beginning of incubation, at 1 hr and 2 hr after incubation, a 200-μL aliquot is taken from the receiver chamber and replaced with fresh assay buffer. The concentration of the compound in each sample is determined with LC/MS/MS. The apparent permeability, Papp, is calculated.

Plasma Protein Binding: Plasma protein binding is measured by equilibrium dialysis. Each compound is spiked into blank plasma at a final concentration of 2 μM. The spiked plasma and phosphate buffer is placed into opposite sides of the assembled dialysis cells, which is then rotated slowly in a 37° C. water bath. At the end of the incubation, the concentration of the compound in plasma and phosphate buffer is determined. The percent unbound is calculated using the following equation:

$$\% Unbound = 100 \cdot ( \frac{{C}_{f}}{{C}_{b} + {C}_{f}} )$$

Where C_{f} and C_{b} are free and bound concentrations determined as the post-dialysis buffer and plasma concentrations, respectively.

CYP450 Profiling: Each compound is incubated with each of 5 recombinant human CYP450 enzymes, including CYP1A2, CYP2C9, CYP3A4, CYP2D6 and CYP2C19 in the presence and absence of NADPH. Serial samples can be taken from the incubation mixture at the beginning of the incubation and at 5, 15, 30, 45 and 60 min after the start of the incubation. The concentration of the compound in the incubation mixture is determined by LC/MS/MS. The percentage of the compound remaining after incubation at each time point is calculated by comparing with the sampling at the start of incubation.

Stability in Rat, Dog, Monkey and Human Plasma: Compounds are incubated for up to 2 hour in plasma (rat, dog, monkey, or human) at 37° C. Compounds are added to the plasma at final concentrations of 1 and 10 μg/mL. Aliquots are taken at 0, 5, 15, 30, 60, and 120 min after adding the compound. Concentration of compounds and major metabolites at each timepoint are measured by LC/MS/MS. Biological data (antiviral potency [EC₅₀] was determined using a Renilla luciferase (RLuc)-based HCV replicon reporter assay—HCV 1b RLuc) for Compound 15 is 0.0045 nM.

TABLE I

| Representive Compound of the Invention | Activity (nM) |
|---|---|
| 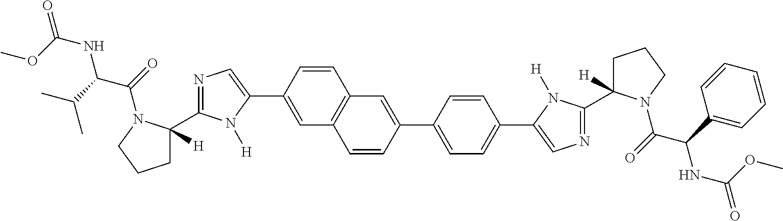 | 0.0081 |
| 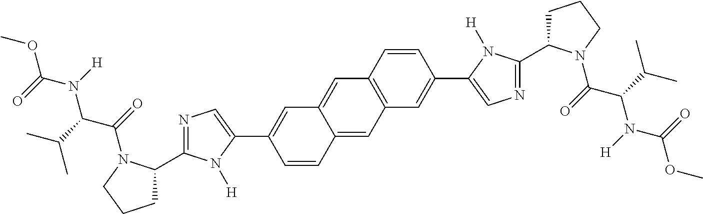 | 0.0070 |
| 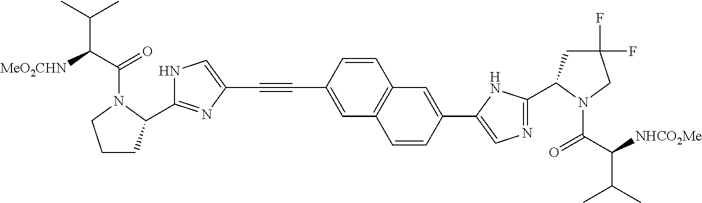 | 0.0147 |
| 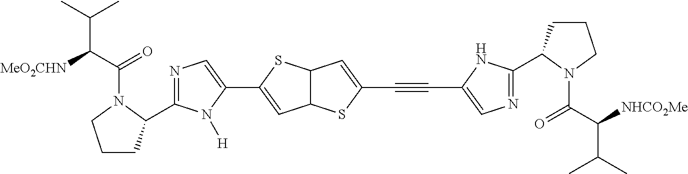 | 0.0503 |
| 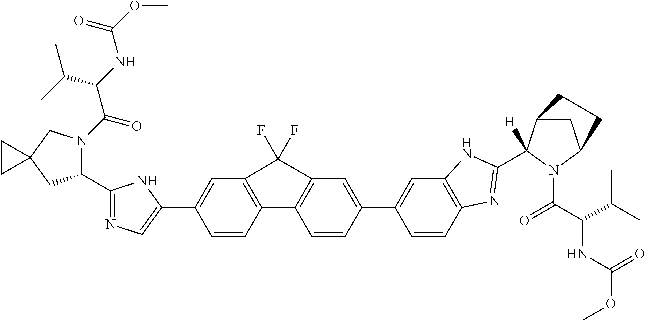 | 0.0045 |
| 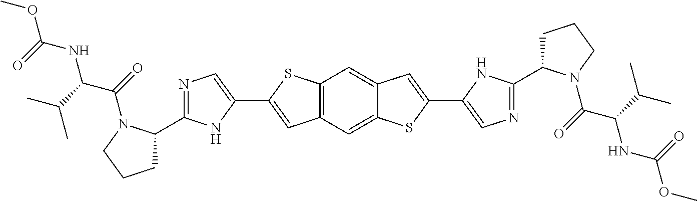 | 0.0077 |

### Biological Example 1

### Anti-HCV Activity of the Combination of Compound 1 and Compound 2

### Materials and Methods

Compound 1 and Compound 2 were synthesized by Gilead Sciences (Foster City, Calif.).

### Cell Lines

HCV genotype 1b replicon cells (Huh-luc) were obtained from Reblikon (Mainz, Germany). The replicon in these cells is designated I389luc-ubi-neo/NS3-3′/ET and encodes a selectable resistance marker (neomycin phosphotransferase) as well as the firefly luciferase reporter gene. Huh-luc cells were maintained in Dulbecco's Modified Eagle's Medium (DMEM; GIBCO, Carlsbad, Calif.) supplemented with 10% fetal bovine serum (FBS; Hyclone, Logan, Utah) and 0.5 mg/mL of G-418 (GIBCO). Cells were passaged twice a week and maintained at subconfluent levels.

### EC₅₀ Determinations

Replicon cells were seeded in 96-well plates at a density of 5×10³ cells per well in 100 μL of DMEM culture medium, excluding G-418. Compounds 1 and 2 were serially diluted 1:3 in 100% DMSO (Sigma). These serial dilutions were added to the cells at a 1:200 dilution to achieve a final concentration of 0.5% DMSO in a total volume of 200 μL. Plates were incubated at 37° C. for 3 days, after which culture media were removed and cells were lysed and assayed for luciferase activity using a commercial luciferase assay (Promega, Madison, Wis.). HCV replication levels in drug-treated samples were expressed as a percentage of those in untreated controls (defined as 100%), and data were fit to the logistic dose response equation y=a/(1+(x/b)c) using XLFit4 software (IDBS, Emeryville, Calif.). EC₅₀ values were calculated from the resulting equations as described previously (Delaney, W. E., et al., Antimicrobial Agents Chemotherapy, 45(6):1705-1713 (2001)).

### Antiviral Combination Studies

Replicon cells were seeded in 96-well plates at a density of 5×10³ cells per well in 100 μL of culture medium. Compounds 1 and 2 were serially diluted in 100% DMSO as described above and added in a matrix format to 96-well plates, achieving a defined set of different drug concentrations and ratios in a final volume of 200 μL and a final DMSO concentration of 0.5%. For each individual drug, the EC₅₀ value was selected as the midpoint for the concentration range tested. Cells were incubated for three days and analyzed for luciferase expression as indicated above. For the combination study, two independent experiments were performed in triplicate.

### Combination Data Analysis

Data were analyzed using the MacSynergy II program developed by Prichard and Shipman (Prichard M N, Aseltine K R, Shipman C, Jr., MacSynergy™ II, Version 1.0. University of Michigan, Ann Arbor, Mich., 1993; Prichard M. N., Shipman C., Jr., Antiviral Res 14 (4-5):181-205 (1990); Prichard M. N., Shipman C, Jr., Antivir Ther 1 (1):9-20 (1996); Prichard M. N., et al., Antimicrob Agents Chemother 37 (3):540-5 (1993). The software calculates theoretical inhibition assuming an additive interaction between drugs (based on the Bliss Independence model) and quantifies statistically significant differences between the theoretical and observed inhibition values. Plotting these differences in three dimensions results in a surface where elevations in the Z-plane represent antiviral synergy and depressions represent antiviral antagonism between compounds. The calculated volumes of surface deviations are expressed in nM²%. Per Prichard and Shipman, combination effects are defined as:
Strong synergy if volumes >100 nM²; this amount of synergy is probably important in vivo
Moderate synergy if volumes are >50 and ≦100 nM²; this amount of synergy may be important in vivo
Minor synergy if volumes are >25 and <50 nM²
Additivity if volumes are >−25 nM² and ≦25 nM²
Minor antagonism if volumes are <−25 and >−50 nM²
Moderate antagonism if volumes are >−100 nM² and ≦−50 nM²; this amount of antagonism may be important in vivo
Strong antagonism if volumes are ≦−100 nM²; this amount of antagonism is probably important in vivo

### Results

Prior to initiating combination experiments, EC₅₀ values in Huh-luc replicon cells were determined for Compound 1 and Compound 2 and results are shown in Table II. Both compounds had an antiviral effect.

TABLE II
Individual EC₅₀s for Anti-HCV Compounds
1 and 2 in Huh-luc Replicon Cells

| Compound | EC₅₀ (nM)^{a} |
|---|---|
| Compound 1 | 3 ± 2 |
| Compound 2 | 11 ± 3 |

^{a}EC₅₀ indicates average ± standard deviation for two or more independent experiments.

The antiviral effect of the combination of Compound 1 and Compound 2 was measured, and the resulting data were analyzed using MacSynergy II, which provides surface plots displaying significant deviations from additivity. Quantification of statistically significant deviations from additivity indicated that the combination of Compounds 1 and 2 had synergy/antagonism volumes between −25 nM² and 25 nM² indicating additive antiviral effects as shown in Table III.

TABLE III
Quantification of Antiviral Synergy and Antagonism and Drug
Interactions for Combination of Compound 1 and Compound 2

| Drug(s) Used in | | | |
|---|---|---|---|
| Combination with | Synergy Volume | Antagonism | |
| Compound 2 | (nM²)^{a} | Volume (nM²)^{a} | Interaction |
| Compound 1 | 13.5 ± 10.5 | 0.07 ± 0.07 | Additive |

^{a}Values represent the mean ± standard deviation of two independent experiments performed in triplicate

The results of the in vitro experiments set forth in Table III indicate that Compound 2 has additive antiviral activity when combined with Compound 1.

### Biological Example 2

### Combinations with Compound 3

### Materials and Methods

### Antiviral Compounds

Compound 1 and Compound 3 were synthesized by Gilead Sciences (Foster City, Calif.). Ribavirin and IFN-α were purchased from Sigma (St. Louis, Mo.).

### Cell Lines

HCV genotype 1b replicon cells (Huh-luc) were obtained from Reblikon (Mainz, Germany). The replicon in these cells is designated I389luc-ubi-neo/NS3-3′/ET and encodes a selectable resistance marker (neomycin phosphotransferase) as well as the firefly luciferase reporter gene. Huh-luc cells were maintained in Dulbecco's Modified Eagle Medium (D-MEM) with GlutaMAX™ (Invitrogen, Carlsbad, Calif.) supplemented with 10% fetal bovine serum (FBS, Hyclone, Logan, Utah) and 0.5 mg/mL of G-418 (Invitrogen). Cells were passaged twice a week and maintained at subconfluent levels.

### EC₅₀ Determinations

Replicon cells were seeded in 96-well plates at a density of 5×10³ cells per well in 100 μL of DMEM plus 10% FBS culture medium, excluding G-418. Compounds were serially diluted 1:3 in 100% DMSO (Sigma). These serial dilutions were added to the cells at a 1:200 dilution to achieve a final concentration of 0.5% DMSO in a total volume of 200 μL. Plates were incubated at 37° C. for 3 days, after which culture media were removed and cells were lysed and assayed for luciferase activity using a commercial luciferase assay (Promega, Madison, Wis.). HCV replication levels in drug-treated samples were expressed as a percentage of those in untreated controls (defined as 100%), and data were fit to the logistic dose response equation y=a/(1+(x/b)^{c}) using XLFit4 software (IDBS, Emeryville, Calif.). EC₅₀ values were calculated from the resulting equations as described previously.

### Antiviral Combination Studies

Replicon cells were seeded in 96-well plates at a density of 5×10³ cells per well in 100 μL of culture medium, excluding G-418. Compound 3 and other compounds were serially diluted in 100% DMSO as described above and added in a matrix format to 96-well plates, achieving a defined set of different drug concentrations and ratios in a final volume of 200 μL and a final DMSO concentration of 0.5%. For each individual drug (with the exception of Ribavirin), the EC₅₀ value was selected as the midpoint for the concentration range tested. For Ribavirin, which did not have a selective antiviral effect, a top dose of 6.2 μM was selected since this was approximately 3-fold below the concentration at which cytotoxicity started to be observed. Cells were incubated with drugs for three days and analyzed for luciferase expression as indicated above. For each combination study, two independent experiments were performed in triplicate.

### Combination Data Analysis

Data were analyzed using the MacSynergy II program developed by Prichard and Shipman. The software calculates theoretical inhibition assuming an additive interaction between drugs (based on the Bliss Independence model) and quantifies statistically significant differences between the theoretical and observed inhibition values. Plotting these differences in three dimensions results in a surface where elevations in the Z-plane represent antiviral synergy and depressions represent antiviral antagonism between compounds. The calculated volumes of surface deviations are expressed in nM²%. Per Prichard and Shipman, combination effects are defined as follows:
Strong synergy if volumes >100 nM²; this amount of synergy is probably important in vivo
Moderate synergy if volumes are >50 and ≦100 nM²; this amount of synergy may be important in vivo
Minor synergy if volumes are >25 and <50 nM²
Additivity if volumes are >−25 nM² and ≦25 nM²
Minor antagonism if volumes are <−25 and >−50 nM²
Moderate antagonism if volumes are >−100 nM² and ≦−50 nM²; this amount of antagonism may be important in vivo
Strong antagonism if volumes are ≦−100 nM²; this amount of antagonism is probably important in vivo

### Results

### EC₅₀ Values for Individual Compounds in Huh-luc Replicon Cells.

Prior to initiating combination experiments, EC₅₀ values in Huh-luc replicon cells were determined for each compound as shown in Table IV. All compounds had an antiviral effect with the exception of Ribavirin, which had no antiviral activity up to concentrations which were beginning to show cytotoxicity.

TABLE IV
Individual EC₅₀s for Anti-HCV Compounds
in Huh-luc Replicon Cells

| Compound | EC₅₀ (nM)^{a} |
|---|---|
| Compound 3 | 2.3 ± 2.6 |
| IFN-α | 0.105 ± .003 (U/mL)^{b} |
| Ribavirin | >12,500 |
| Compound 1 | 0.4 ± 0.14 |

^{a}EC₅₀ indicates average ± standard deviation for two or more independent experiments.
^{b}INF-α EC₅₀ is expressed in Units (U) per milliliter (mL) instead of a nanomolar concentration.

### Combination Antiviral Effects and Drug Interactions

The antiviral effects of Compound 3 when combined with IFN-α, Ribavirin, and Compound 1 were assayed. The resulting data were analyzed using MacSynergy II, which provides surface plots displaying significant deviations from additivity. Quantification of statistically significant deviations from additivity indicated that combinations of Compound 3 with IFN-α resulted in minor synergy (synergy volumes of 32 and 36.5 nM², respectively; Table V). The combination of Compound 3 with the non-nucleoside NS5B inhibitor Compound 1 yielded an synergy volume of 14.5 nM² which indicates an additive antiviral interaction. None of the compounds yielded antiviral antagonism volumes outside of the additive range (>−25 nM²) when combined with Compound 3 as shown in Table V.

TABLE V
Quantification of Antiviral Synergy and Antagonism and
Drug Interactions for Drug Combinations with Compound 3

| Drug(s) Used in | | | |
|---|---|---|---|
| Combination with | Synergy | Antagonism | |
| Compound 3 | Volume (nM²)^{a} | Volume (nM²)^{a} | Interaction |
| IFN-α | 32 ± 4.2 | 0.15 ± 0.2 | Minor synergy |
| Ribavirin | 54 ± 14.1 | 1.6 ± 2.3 | Moderate synergy |
| Compound 1 | 14.5 ± 0.7 | 4.22 ± 5.0 | Additive |

^{a}Values represent the mean ± standard deviation of two independent experiments performed in triplicate

These in vitro antiviral combination experiments indicate that the novel HCV NS3 protease inhibitor Compound 3 has minor synergy when combined with IFN-α and moderate synergy when combined with Ribavirin. These results suggest that Compound 3 could potentially be used in combination with the current standard of care (PEG-IFN-α plus ribavirin) in HCV patients to achieve enhanced viral load suppression without reducing the efficacy of any of the individual drugs. Combinations of Compound 3 with non-nucleoside (Compound 1) NS5B polymerase inhibitors resulted in additivity. These results indicate that Compound 3 may also be suitable for exploring drug combinations comprised of multiple classes of specific HCV inhibitors in patients.

### Clinical Example 1

### Clinical Testing of Anti-HCV Activity of the Combination of Compound 1 and Compound 2

This Clinical Example shows that the combination of Compound 1 and Compound 2 plus ribavirin is more effective at reducing HCV viral load, and suppressing HCV viral rebound, than the combination of Compound 1 plus Compound 2 without ribavirin.

### Clinical Trial Design:

A Phase 2, randomized, open-label trial of Compound 2 plus Compound 1 alone and in combination with ribavirin for 28 days in treatment-naive subjects with chronic genotype 1 HCV infection. Subjects in Arm 1 received Compound 2 at 75 mg+Compound 1 at 40 mg, both administered twice daily (BID) (double regimen) and subjects in Arm 2 received Compound 2 at 75 mg+Compound 1 at 40 mg, both administered BID, and plus ribavirin, also administered BID (triple regimen) for 28 days.

On Day 28, all subjects were to initiate PEG/Ribavirin. Additionally, the protocol called for subjects with an insufficient virologic response (<2 log₁₀ IU/mL reduction from baseline HCV RNA by Day 5) or virologic rebound (HCV RNA increase of >0.5 log₁₀ IU/mL from nadir confirmed over two time points occurring after Day 5 with an absolute value >1000 IU/mL) to initiate PEG/RIBA prior to Day 28.

For subjects with insufficient virologic response, the combination of pegylated interferon (PEG) and ribavirin (RIBA) was initiated prior to Day 28 with or without continuation Compound 2+Compound 1. As a result, by Day 28 of the study, subjects were receiving one of four treatments:
(i) Compound 2+Compound 1,
(ii) Compound 2+Compound 1+RIBA,
(iii) Compound 2+Compound 1+PEG/RIBA, or
(iv) PEG/RIBA.

A total of 31 subjects were enrolled and started dosing (16 subjects received the double regimen in Arm 1 and 15 subjects received the triple regimen in Arm 2). Preliminary subject demographics and baseline characteristics (Table VI) were generally comparable between Arms 1 and 2, aside from a greater number of subjects with genotype 1b in Arm 2. Four subjects were identified as HCV genotype 1b at screening (one subject on the dual regimen and three subjects on the triple regimen), but have not been confirmed as genotype 1a or 1b upon further analysis, with further assessment ongoing.

No subjects have experienced serious adverse events. Study medications have been generally well-tolerated, with all adverse events being Grade 1-2 in severity, except for a single Grade 3 fatigue, which was the only treatment emergent adverse event leading to study drug discontinuation. Prior to the initiation of PEG/Ribavirin, the most common treatment-emergent adverse events occurring in more than one subject were headache (n=5), and diarrhea or nausea (n=3 each) in Arm 1 and headache (n=7), diarrhea or fatigue (n=3 each), nausea, asthenia, pruritis or insomnia (n=2 each) in Arm 2. When Compound 2+Compound 1 were given in combination with PEG/RIBA, the only adverse events occurring in more than one subject were influenza-like illness (n=5) and myalgia (n=3), both common adverse events with PEG/RIBA therapy. With regard to laboratory abnormalities, there were no Grade 4 events during the 28-day treatment period. Among subjects receiving the study drugs, there were two treatment-emergent Grade 3 elevations in total bilirubin in the ribavirin containing Arm 2 (occurring at Day 7, but resolving with continued dosing of study drug). There were also 2 Grade-1 elevations and a single Grade-2 elevation in total bilirubin among other subjects in this dosing Arm (with ribavirin). Among subjects in Arm-1 (no ribavirin), there were four Grade-1 total bilirubin elevations. ALT values were reduced approximately 40 U/L from baseline in both arms by Day 14. Median QTcF was not significantly changed from baseline in either study arm and no subjects discontinued study drugs due to QTc abnormalities. Preliminary safety data are summarized in Table VII.

Plasma HCV RNA was monitored approximately twice weekly to gauge virologic response in relation to the protocol-specified criteria for early initiation of PEG/RIBA. From preliminary analysis of the HCV RNA values, the median maximal decline in HCV RNA was 3.9 log₁₀ IU/mL for the dual regimen and 5.0 log₁₀ IU/mL for the triple regimen. The median time to maximal decline in HCV RNA was 7 days for the dual regimen and 14 days for the triple regimen, with the difference attributed to delayed incidence and onset of viral breakthrough in the ribavirin containing arm. Three of 15 (20%) subjects receiving the dual regimen and 10 of 13 (77%) subjects receiving the triple regimen had nadir HCV RNA values ≦30 IU/mL (excluding non-GT1 subjects). 13/16 (81%) subjects receiving Compound 2/Compound 1 and 6/15 (40%) subjects receiving Compound 2/Compound1/Ribavirin initiated PEG or PEG/Ribavirin prior to the scheduled start on Day 28 of the study. Additional details of virologic outcomes are provided in Results. Compound 2+Compound 1 alone and in combination with RIBA were well-tolerated for up to 28 days by HCV subjects in this study, both before and following the addition of PEG or PEG/Ribavirin. Both regimens yielded potent suppression of HCV RNA, with greater and more sustained activity in the three drug regimen.

TABLE VI
Preliminary Subject Demographics and Baseline Characteristics

| | Arm #1: | | Arm #2: | |
|---|---|---|---|---|
| | Compound 2 at 75 mg | | Compound 2 at 75 mg | |
| | BID + Compound 1 at | | BID + Compound 1 at | |
| | 40 mg BID | | 40 mg BID + RIBA | |
| | (n = 16) | | (n = 15) | |
| Age in years - Median (range) | 47 | | 55 | |
| | (30, 66) | | (27, 63) | |
| Sex | 14 | male | 11 | male |
| | 2 | female | 4 | female |
| Ethnicity | 16 | Non-Hispanic/Latino | 15 | Non-Hispanic/Latino |
| Race | 13 | White | 13 | White |
| | 2 | Black | 2 | Black |
| | 1 | Asian | 0 | Asian |

| | | |
|---|---|---|
| Baseline Weight in kg - Median | 86.1 | 79.0 |
| (range) | (57.8, 110.5) | (51, 127.5) |
| Baseline BMI in kg/M² - Median | 27.1 | 24.7 |
| (range) | (21.5, 34.1) | (19.9, 37.6) |
| Baseline Log₁₀ HCV RNA (IU/mL) | 6.17 | 6.34 |
| from Central lab- Median (range) | (5.25, 7.26) | (5.41, 7.19) |
| Central lab | | |
| Baseline HCV Genotype | 8 1a | 3 1a |
| | 8 1b | 12 1b |

TABLE VII
Preliminary Safety Results

| | Arm 1: | Arm 2: |
|---|---|---|
| | Compound 2 at 75 mg | Compound 2 at 75 mg |
| | BID + Compound 1 at | BID + Compound 1 at |
| | 40 mg BID | 40 mg BID + RIBA |
| | (n = 16) | (n = 15) |
| Grade 3 Adverse | | |
| Events (AEs): | | |
| Fatigue | 1 | 0 |
| Grade 1/Grade 2 | | |
| (AEs): | | |
| Headache | 5 (31%) | 7 (47%) |
| Diarrhea | 3 (19%) | 3 (20%) |
| Nausea | 3 (19%) | 2 (13%) |
| Fatigue | 0 | 3 (20%) |
| Asthenia | 0 | 2 (13%) |
| Pruritis | 1 (6%) | 2 (13%) |
| Insomnia | 0 | 2 (13%) |
| Grade 3 Laboratory | | |
| Abnormalities: | | |
| Bilirubin | 0 | 2 |
| Grade 1/Grade 2 | | |
| Laboratory | | |
| Abnormalities: | | |
| Bilirubin | 4 | 3 |
| Hemoglobin | 0 | 2 |
| Glucose (nonfasting) | 8 | 5 |

TABLE VIII
Preliminary Virologic Outcomes

| | | | | | Arm 2: | | Arm 2: | |
|---|---|---|---|---|---|---|---|---|
| | Arm 1: | | Arm 1: | | Compound 2 at | | Compound 2 at 75 mg BID + | |
| | Compound 2 at | | Compound 2 at 75 mg BID + | | 75 mg BID + | | Compound 1 at 40 mg BID + | |
| | 75 mg BID + | | Compound 1 at 40 mg BID | | Compound 1 at | | Ribavirin | |
| | Compound 1 at | | Unconfirmed | | 40 mg BID + | | Unconfirmed | |
| | 40 mg BID | | GT1 Subjects Excluded | | Ribavirin | | GT1 Subjects Excluded | |
| | (n = 16) | | (n = 15)* | | (n = 15) | | (n = 13) | |
| Median maximal | −3.9 log₁₀ | IU/mL | −4.0 log₁₀ | IU/mL | −5.0 log₁₀ | IU/mL | −5.0 log₁₀ | IU/mL |
| HCV RNA | | | | | | | | |
| decline | | | | | | | | |
| Mean maximal | −3.4 log₁₀ | IU/mL | −3.6 log₁₀ | IU/mL | −4.5 log₁₀ | IU/mL | −4.9 log₁₀ | IU/mL |
| HCV RNA | | | | | | | | |
| decline | | | | | | | | |
| Mean time to | 16 | days | 16 | days | 23 | days | 23 | days |
| Breakthrough | | | | | | | | |
| Subjects with | 3/16 | (19%) | 3/15 | (20%) | 10/15 | (63%) | 10/13 | (77%) |
| HCV RNA | | | | | | | | |
| nadir <50 IU/mL | | | | | | | | |
| Subjects with | 12 | (75%) | 12/15 | (80%) | 6/15 | (40%) | 6/13 | (46%) |
| Breakthrough** | | | | | | | | |
| Day 28 | | | | | | | | |
| Response: | | | | | | | | |
| RVR at <25 | 1/16 | (6%) | 1/15 | (7%) | 5/15 | (33%) | 5/13 | (38%) |
| IU/mL | | | | | | | | |
| RVR at <50 | 1/16 | (6%) | 1/15 | (7%) | 6/15 | (40%) | 6/13 | (46%) |
| IU/mL | | | | | | | | |

*GT1 is an abbreviation for HCV Genotype 1.
Subjects 1011, 1012, and 1043 at one French study center were excluded;
Subject 1004 was not excluded
**Breakthrough defined as >1 log increase in HCV RNA above nadir value or HCV RNA >25 IU/mL following a nadir of <25 IU/mL

The data presented in Table VIII show that there was an approximately 10 fold greater decline in both the median maximal HCV RNA level and the mean maximal HCV RNA level in response to the combination of Compound 2+Compound 1 in the presence of ribavirin compared to the absence of ribavirin. Also, the number of study subjects having an HCV RNA nadir below 50 IU/mL is greater in the presence of ribavirin than in the absence of ribavirin. These results show that ribavirin, in the absence of interferon, significantly potentiates the antiviral activity of the combination of Compound 1 and Compound 2.

Additionally, the mean time to HCV breakthrough, which is a measure of the eventual increase in HCV viral load as the virus mutates and becomes less susceptible to the antiviral drugs, is greater in the presence of ribavirin than in the absence of ribavirin. Further, the number of subjects showing viral breakthrough is substantially less in the presence of ribavirin than in the absence of ribavirin. These results show that the HCV virus is less able to develop resistance to the combination of Compound 1 and Compound 2 in the presence of ribavirin.

Further, the data presented in Table VIII shows that the number of patients achieving a Rapid Virologic Response (RVR) in the presence of ribavirin is significantly greater than in the absence of ribavirin. Achievement of RVR positively correlates with cure of HCV infection.

Taken together the data presented in Table VIII show that the combination of Compound 1, Compound 2, and ribavirin causes a rapid and clinically significant reduction in HCV viral load, with a reduced viral rebound, even in the absence of administration of interferon.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A dosing regimen for the treatment of HCV comprising:
a. administering one or more anti-HCV compounds or a pharmaceutically acceptable salts thereof; and
b. ribavirin, but not one or more interferon.

2. A method for ameliorating one or more symptom of HCV infection in a human comprising:
a. administering one or more anti-HCV compounds or a pharmaceutically acceptable salts thereof; and
b. ribavirin,
without concurrent administration of one or more interferon.

3. A method for reducing viral load in a human diagnosed with HCV comprising:
a. administering one or more anti-HCV compounds or a pharmaceutically acceptable salts thereof; and
b. ribavirin, but not one or more interferon.

4. A method for treating HCV in a human subject consisting essentially of administration of ribavirin in conjunction with one or more anti-HCV compounds or a pharmaceutically acceptable salts thereof.

5. A method of ribavirin-based HCV therapy comprising:
a. administering one or more anti-HCV compounds or a pharmaceutically acceptable salts thereof; and
b. avoiding administration of one or more interferon.

6. A method for reducing emergence of HCV quasispecies with resistance to coadministered oral antiviral agents comprising:
a. administering one or more anti-HCV compounds or a pharmaceutically acceptable salts thereof; and
b. ribavirin,
without concurrent administration of one or more interferon.

7. A composition for ameliorating one or more symptom of HCV infection in a human comprising:
a. one or more anti-HCV compounds or a pharmaceutically acceptable salts thereof; and
b. ribavirin,
without one or more interferon.

8. A composition for reducing viral load in a human diagnosed with HCV comprising:
a. one or more anti-HCV compounds or a pharmaceutically acceptable salts thereof; and
b. ribavirin, but not one or more interferon.

9. A composition for treating HCV in a human subject consisting essentially of ribavirin in conjunction with one or more anti-HCV compounds or a pharmaceutically acceptable salts thereof.

10. A composition for ribavirin-based HCV therapy comprising:
one or more anti-HCV compounds or a pharmaceutically acceptable salts thereof,
with the proviso that said composition does not include one or more interferon.

11. A composition for reducing emergence of HCV quasispecies with resistance to coadministered oral antiviral agents comprising:
a. one or more anti-HCV compounds or a pharmaceutically acceptable salts thereof; and
b. ribavirin,
without one or more interferon.

12. A combination comprising
a. ribavirin; and
b. one or more anti-HCV compounds or a pharmaceutically acceptable salts thereof;
which is substantially free of one or more interferon.

13. A kit comprising:
a. ribavirin;
b. one or more anti-HCV compounds; and
c. instructions regarding a treatment regimen to treat, reduce viral load, or delay onset or progression of HCV without administration of one or more interferon.

14. A pharmaceutical composition comprising:
a. ribavirin;
b. one or more anti-HCV compounds or a pharmaceutically acceptable salts thereof; and
c. one or more pharmaceutically acceptable carrier.

15. The dosing regimen of claim 1, wherein the one or more anti-HCV compounds are selected from the group consisting of an NS3 protease inhibitor, an NS4B inhibitor, a nucleoside NS5B polymerase inhibitor, a nonnucleoside NS5B polymerase inhibitor, an NS5A inhibitor, and an HCV entry inhibitor.

16. The dosing regimen of claim 1, wherein one of the one or more anti-HCV compounds is Compound 1: 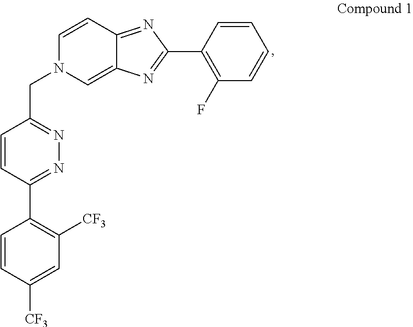
or a pharmaceutically acceptable salt thereof.

17. The dosing regimen of claim 1 comprising at least two anti-HCV compounds or pharmaceutically acceptable salts thereof.

18. The dosing regimen of claim 1, wherein one of the one or more anti-HCV compounds is Compound 2: 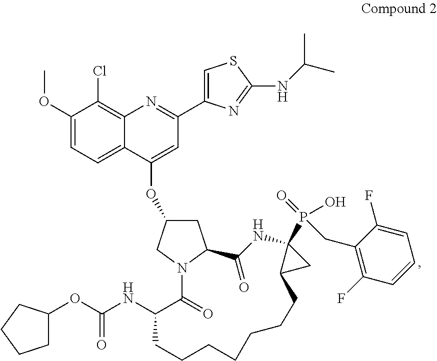
or a pharmaceutically acceptable salt thereof.

19. The dosing regimen of claim 1, wherein the one or more anti-HCV compounds comprise one or more of Compounds 1-17 or any combination thereof.

20. The dosing regimen of claim 1, wherein the one or more anti-HCV compounds comprise Compound 1 and Compound 2 or pharmaceutically acceptable salts thereof.

21. The dosing regimen of claim 1, wherein the one or more anti-HCV compounds comprise Compound 1 and Compound 3 or pharmaceutically acceptable salts thereof.
